(19) United States
(12) Patent Application Publication
(10) Pub. No.: US 2010/0157202 A1
(43) Pub. Date: Jun. 24, 2010

(54) CONJUGATED POLYMER COMPOUND AND POLYMER LIGHT EMITTING DEVICE USING THE SAME

(75) Inventors: Shigeya Kobayashi, Ibaraki (JP); Satoshi Kobayashi, Ibaraki (JP)

Correspondence Address: SUGHRUE MION, PLLC, 2100 PENNSYLVANIA AVENUE, N.W., SUITE 800, WASHINGTON, DC, 20037, US

(73) Assignee: SUMITOMO CHEMICAL COMPANY, LIMITED, Chuo-ku (JP)

(21) Appl. No.: 12/090,497

(22) PCT Filed: Nov. 9, 2006
(86) PCT No.: PCT/JP2006/322797

§ 371 Date: Apr. 29, 2008

(30) Foreign Application Priority Data
Nov. 11, 2005 (JP) 2005-327162

(51) Int. Cl. G02F 1/13357; C08F 32/08; C07C 69/76; C07C 33/05; C07C 13/547; C07C 25/22; H01B 1/12; C09K 11/06; H01J 1/63

(52) U.S. Cl. 349/69; 526/280; 560/100; 568/808; 585/21; 570/183; 252/500; 252/301.35; 313/504

(57) ABSTRACT

A polymer compound which is useful as a light emitting material and a charge transporting material and excellent in electron injection property, comprising as a partial structure therein a structure of the following formula (a) is provided:

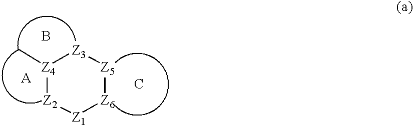

(wherein, a ring A and a ring B represent each independently an aromatic ring or a non-aromatic ring, and at least one of the ring A and the ring B is an aromatic ring. A ring C represents an aromatic ring. Z₁ represents an atom selected from a carbon atom, oxygen atom, sulfur atom, nitrogen atom, silicon atom, boron atom, phosphorus atom and selenium atom or a group containing the atom, and Z₂ to Z₆ represent each independently an atom selected from a carbon atom, silicon atom, nitrogen atom and boron atom or a group containing the atom.).

### TECHNICAL FIELD

The present invention relates to a conjugated polymer compound and a polymer light emitting device (polymer LED) using the same.

### BACKGROUND ART

Light emitting material and charge transporting material of high molecular weight are soluble in solvent and capable of forming an organic layer in a light emitting device by an application method, thus, various investigations have been underwent, polymer compound having as a repeating unit such as the following structure in which two benzene rings are fused to a cyclopentadiene ring are known as an example thereof (for example, Advanced Materials, 1999, vol. 9, No. 10, p. 798; International Publication WO 99/54385 pamphlet). However, electron injection property and the like of the above-described conjugated polymer compounds are not necessarily sufficient.

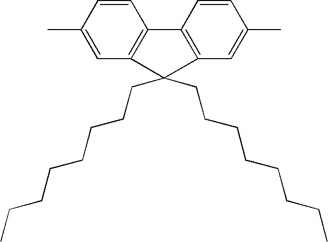

### DISCLOSURE OF THE INVENTION

An object of the present invention is to provide a polymer compound which is useful as a light emitting material and a charge transporting material and excellent in electron injection property.

That is, the present invention provides a conjugated polymer compound comprising as a partial structure therein a structure of the following formula (a):

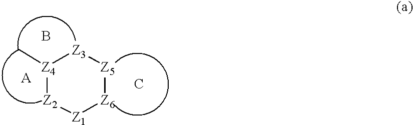

(wherein, a ring A and a ring B represent each independently an aromatic ring optionally having a substituent or a non-aromatic ring optionally having a substituent, and at least one of the ring A and the ring B is an aromatic ring. A ring C represents an aromatic ring optionally having a substituent. Z₁ represents an atom selected from a carbon atom, oxygen atom, sulfur atom, nitrogen atom, silicon atom, boron atom, phosphorus atom and selenium atom or a group containing the atom, and Z₂ to Z₆ represent each independently an atom selected from a carbon atom, silicon atom, nitrogen atom and boron atom or a group containing the atom. When the ring B and the ring C have a substituent, these substituents may be connected to form a ring.).

### MODES FOR CARRYING OUT THE INVENTION

The following formula (1) shows a structure in which Z₄ to Z₆ represent a carbon atom in the above-described formula (a):

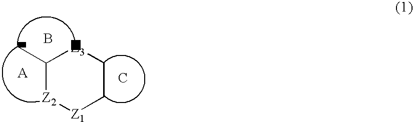

(wherein, the definitions of a ring A, ring B, ring C, Z₁, Z₂ and Z₃ are the same as described above.).

In the formula (1), Z₁ is preferably —C(R_{w})(Rₓ)—, >C═C(R_{w})(Rₓ), —O—, —S—, —S(═O)—, —S(═O)(═O)—, —N(R_{w})—, —Si(R_{w})(Rₓ)—, —P(═O)(R_{w})—, —P(R_{w})—, —B(R_{w})—, —C(R_{w})(Rₓ)—O—, —C(═O)—O—, —C(R_{w})═N— or —Se—, and preferably —C(R_{w})(Rₓ)—, >C═C(R_{w})(Rₓ) or —Si(R_{w})(Rₓ)—.

Here, R_{w} and Rₓ represent each independently a hydrogen atom, or a substituent such as an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, nitro group, amide group, acid imide group, monovalent heterocyclic group, carboxyl group, substituted carboxyl group or cyano group and the like.

In the formula (1), R_{w} and Rₓ are preferably selected from an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, nitro group, amide group, acid imide group, monovalent heterocyclic group, carboxyl group, substituted carboxyl group or cyano group, and further preferably selected from an alkyl group, alkoxy group, alkylthio group, aryl group, aryloxy group, arylthio group, arylalkyl group, arylalkoxy group, arylalkylthio group, arylalkenyl group and arylalkynyl group.

From the standpoint of solubility, R_{w} and Rₓ are further preferably selected from an alkyl group and arylalkyl group.

From the standpoints of heat resistance and light emitting property, Z₁ has preferably at least one substituent, more preferably two substituents, in the formula (1).

In the formula (1), Z₂ and Z₃ represent each independently an atom selected from a carbon atom, silicon atom, nitrogen atom and boron atom or a group containing the atom. The example thereof is preferably >CH—, >CR′—, >C═, >SiH—, or nitrogen (>N—), and it is more preferable from the standpoints of heat resistance and light emitting property that all of them are selected from >CH—, >CR′— and >C═. Here, R′ represents an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, nitro group, amide group, acid imide group, monovalent heterocyclic group, carboxyl group, substituted carboxyl group or cyano group and the like.

The aromatic ring optionally having a substituent in the formula (1) includes aromatic hydrocarbon rings and aromatic heterocyclic rings. As the aromatic hydrocarbon ring, preferable are those in which a benzene ring alone or two or more benzene rings are fused, and examples thereof include aromatic hydrocarbon rings such as a benzene ring, naphthalene ring, anthracene ring, tetracene ring, pentacene ring, pyrene ring, phenanthrene ring and the like, and preferably a benzene ring, naphthalene ring, anthracene ring and phenanthrene ring. The aromatic heterocyclic ring includes a pyridine ring, pyridazine ring, pyrimidine ring, triazine ring, quinoline ring, phenanthroline ring, furan ring, benzofuran ring, thiophene ring, benzothiophene ring, pyrrole ring, indole ring, thiazole ring, oxazole ring and the like.

The non-aromatic ring optionally having a substituent includes aliphatic hydrocarbon rings and non-aromatic heterocyclic rings. The aliphatic hydrocarbon ring includes a cyclopentane ring, cyclohexane ring, cycloheptane ring, cyclononane ring and the like. The non-aromatic heterocyclic ring includes a tetrahydrofuran ring, tetrahydrothiofuran ring, pyrrolidine ring, phosphorolane ring, silolane ring, borolane ring, tetrahydropyran ring, tetrahydrothiopyran ring, piperidine ring, phosphinane ring, borinane ring, silinane ring and the like.

It is preferable that all of elements constituting a ring A, ring B and ring C represent a carbon atom, in a structure of the formula (1).

From the standpoint of a light emitting property, it is preferable that all of a ring A, ring B and ring C represent an aromatic hydrocarbon ring, and it is preferable that these are selected each independently from a benzene ring optionally having a substituent, a naphthalene ring optionally having a substituent, and an anthracene ring optionally having a substituent. It is more preferable that a ring A, ring B and ring C represent each independently a benzene ring or naphthalene ring, and it is further preferable that all of a ring A, ring B and ring C represent a benzene ring.

There are a case in which one molecule of the structure of the formula (1) is contained in the main chain of a conjugated polymer compound, a case in which the structure is contained as a repeating unit, and a case in which the structure is contained in the side chain. From the standpoint of device properties such as heat resistance, solubility, light emitting property, brilliance half life and the like, it is preferable that the structure is contained as a repeating unit in a conjugated polymer compound.

The conjugated polymer compound of the present invention includes one containing the structure of the formula (1) as a repeating unit.

An example thereof includes structures of the following formula (2):

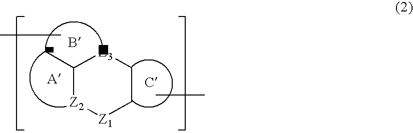

(wherein, a ring B′ and a ring C′ represent each independently an aromatic ring optionally having a substituent, and a ring A′ represents an aromatic ring optionally having a substituent or a non-aromatic ring optionally having a substituent. Two bonds are present on the ring B′ and the ring C′. Z₁ to Z₃ represent the same meanings as described above.).

Other examples include structures of the following formula (3):

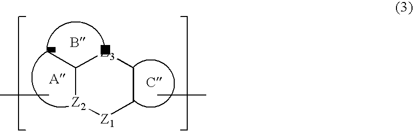

(wherein, a ring A″ and a ring C″ represent each independently an aromatic ring optionally having a substituent, and a ring B″ represents an aromatic ring optionally having a substituent or a non-aromatic ring optionally having a substituent. Two bonds are present on the ring A″ and the ring C″. Z₁ to Z₃ represent the same meanings as described above.).

The definitions, examples and the like of the aromatic ring optionally having a substituent and the non-aromatic ring optionally having a substituent of the formulae (2) and (3) are the same as the definitions and examples of those of said formula (1).

When the conjugated polymer compound of the present invention contains structures of the formula (2) and/or the formula (3) as a repeating unit, the amount of these repeating units in the conjugated polymer compound of the present invention is usually 1 mol % or more and 100 mol % or less, preferably 20 mol % or more, and further preferably 50 mol % or more and 100 mol % or less based on the sum of all repeating units.

Examples of the structures of the formulae (2) and (3) in which Z₂ and Z₃ are other than carbon include those of the following formulae (1V-1) to (1V-9) and these structures having a substituent. In the following structures, bonds on aromatic hydrocarbon rings can take any position.

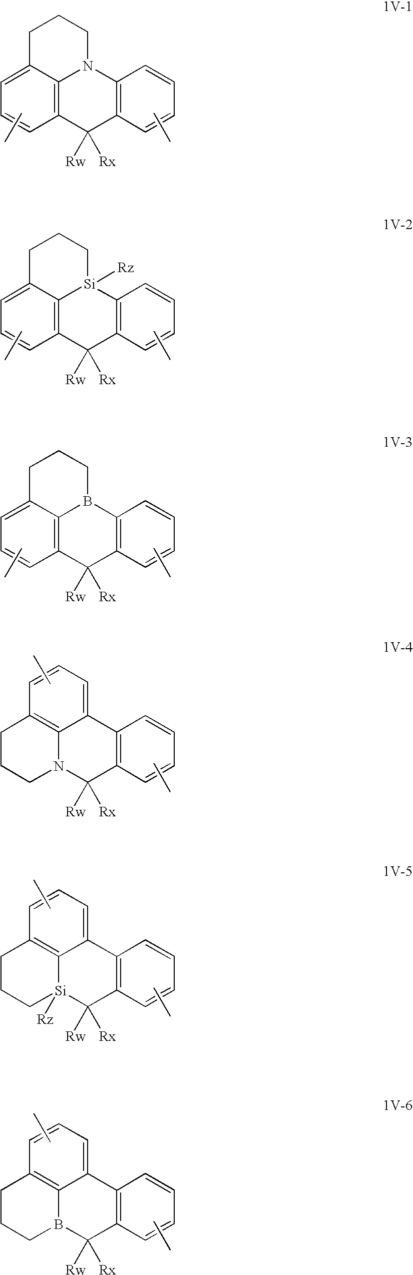

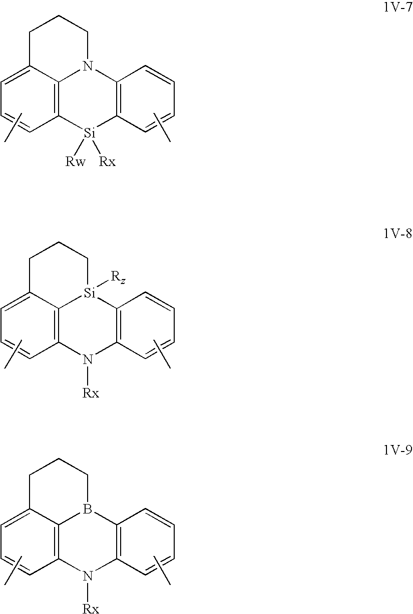

Examples of the structures of the formulae (2) and (3) in which Z₂ is carbon include those of the following formulae (1A-1) to (1J-12) and these structures having a substituent. In the following structures, bonds on aromatic hydrocarbon rings can take any position.

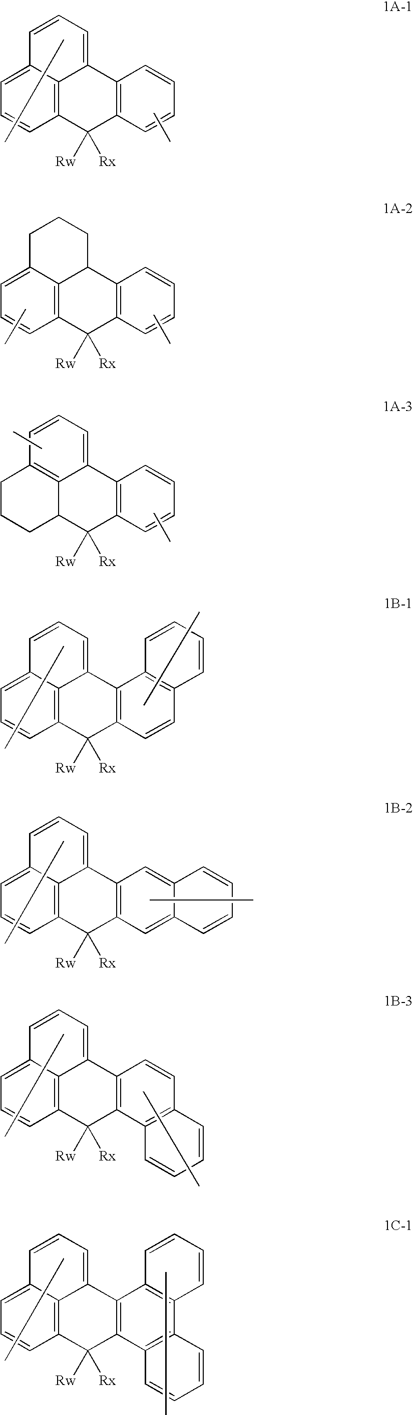

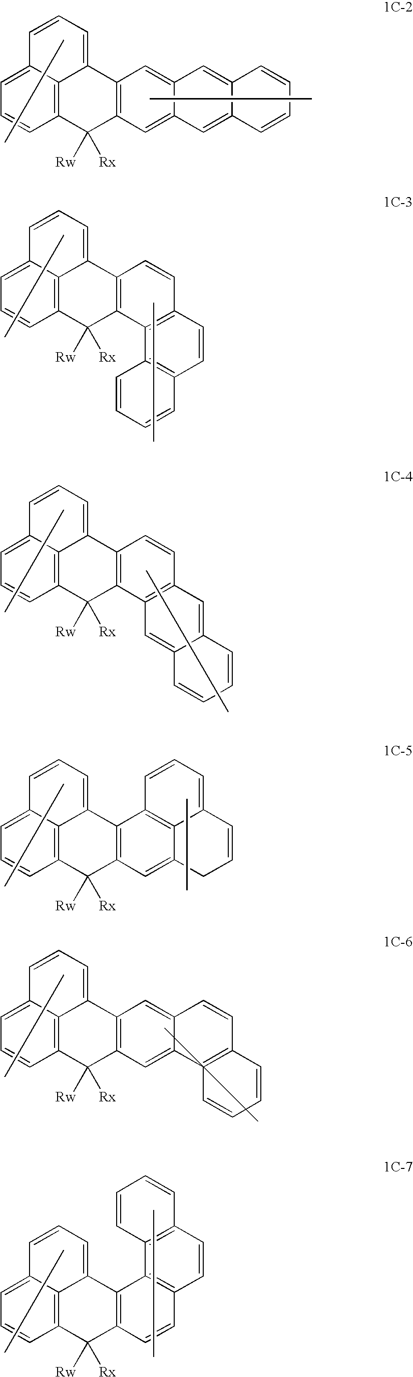

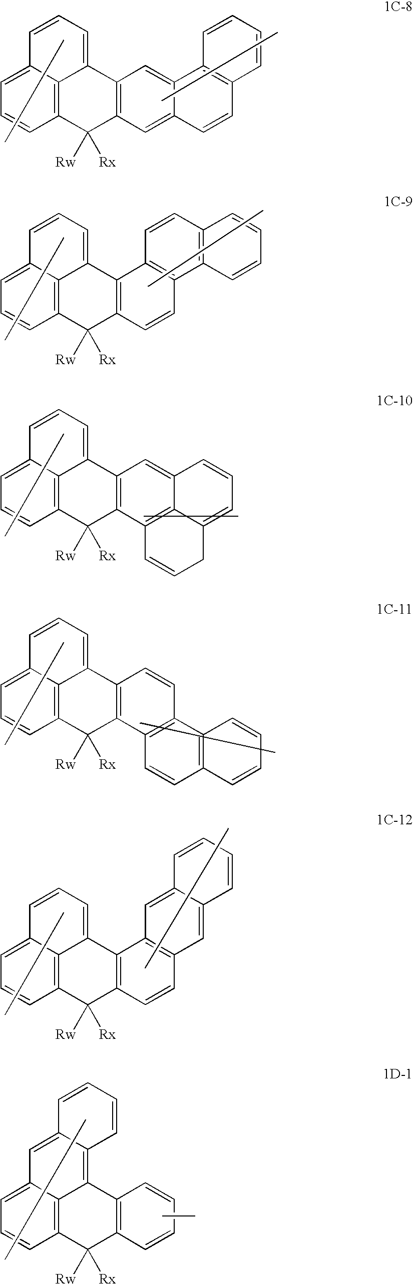

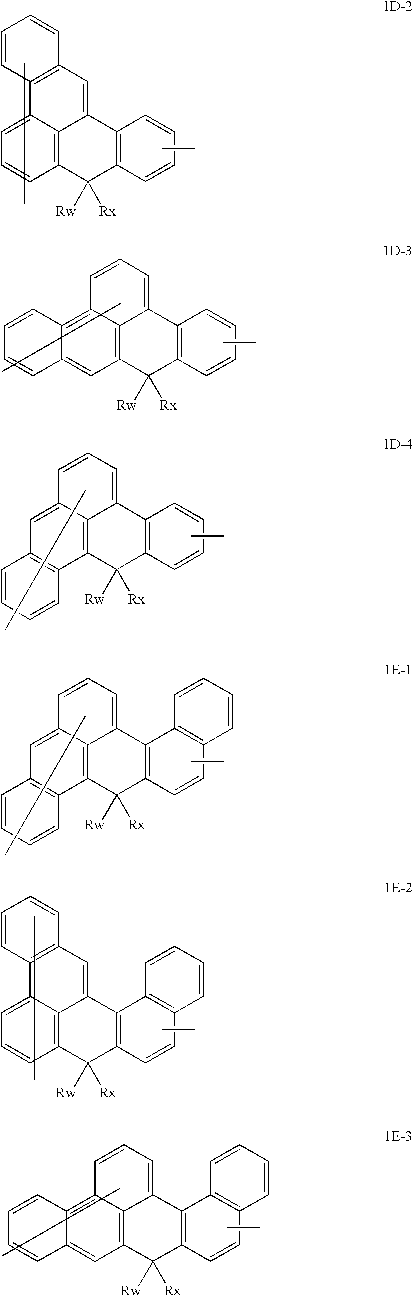

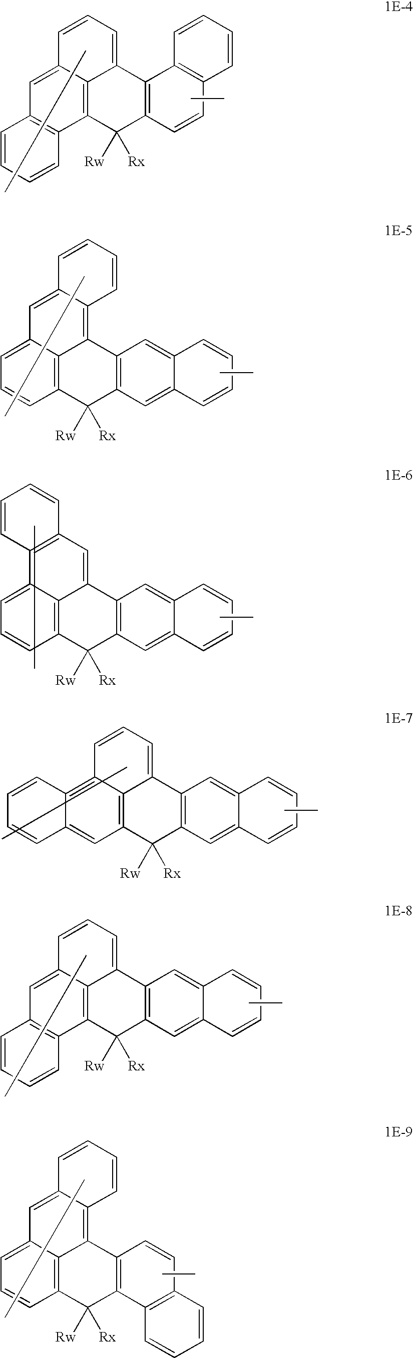

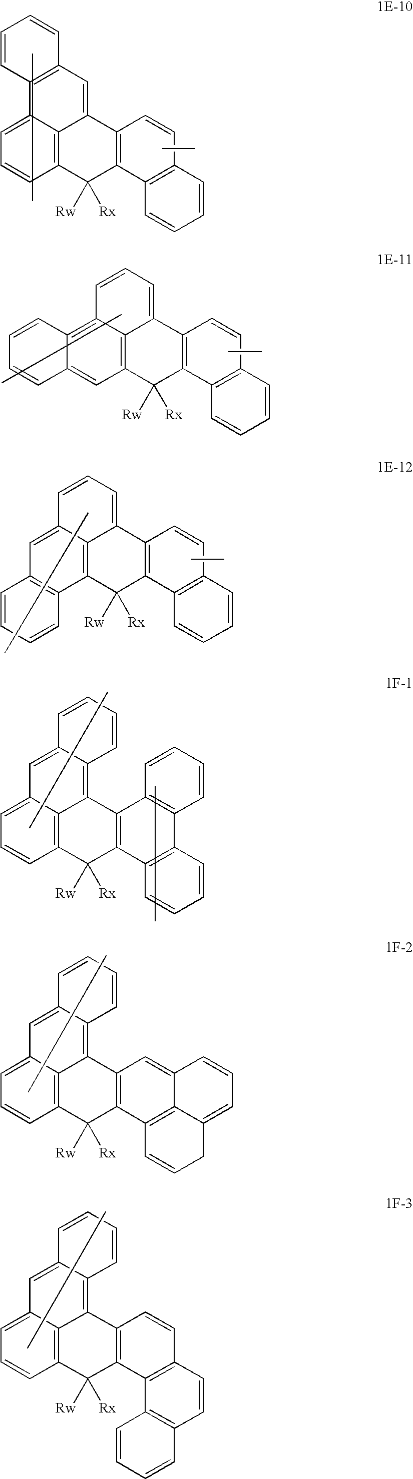

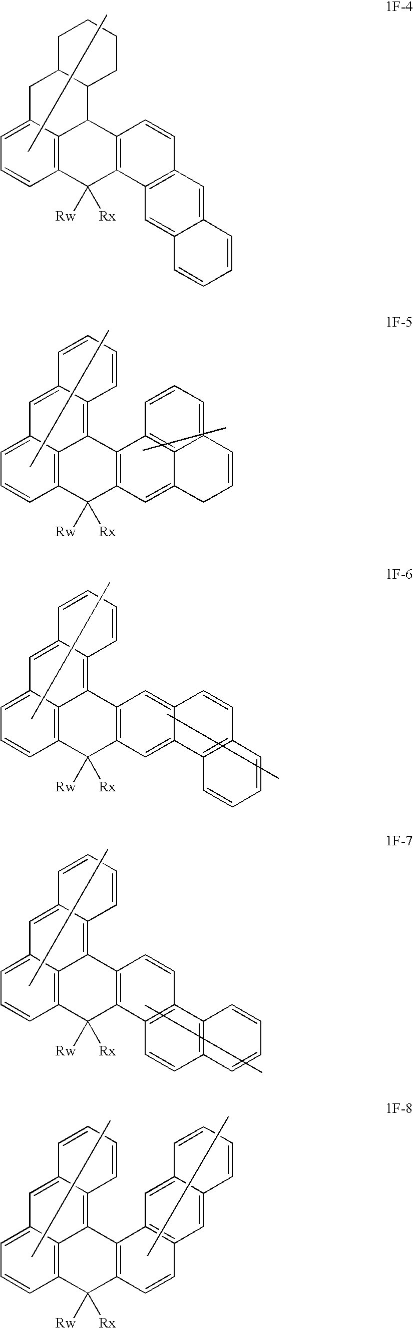

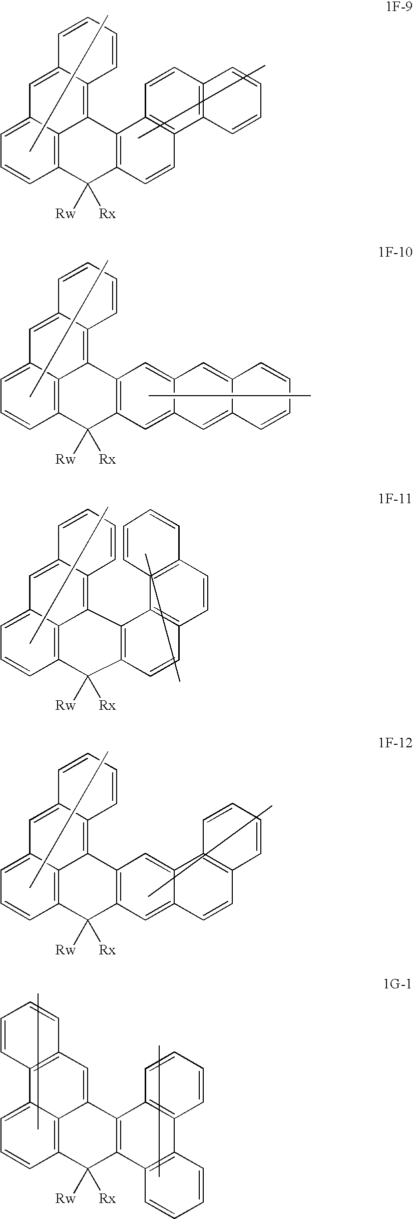

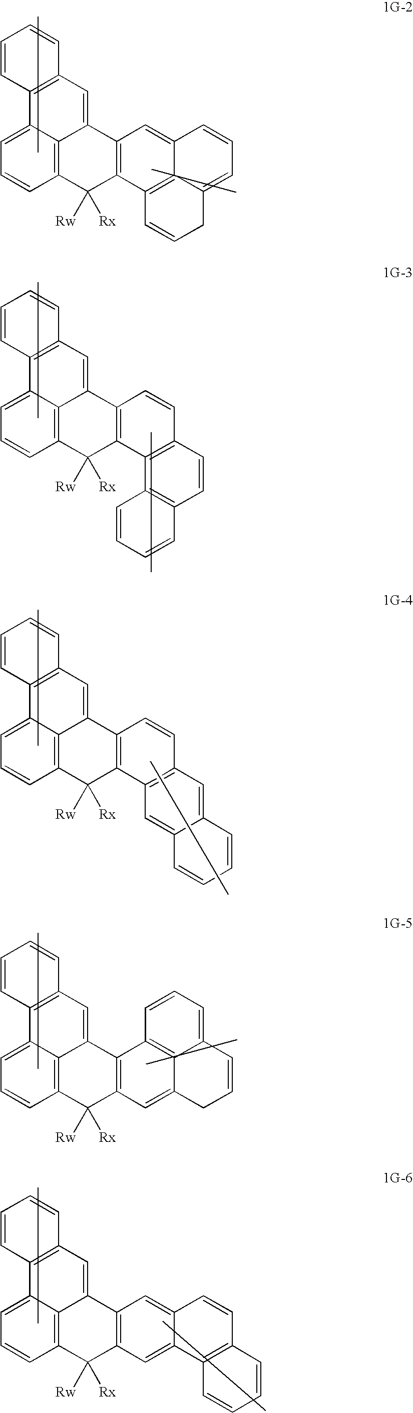

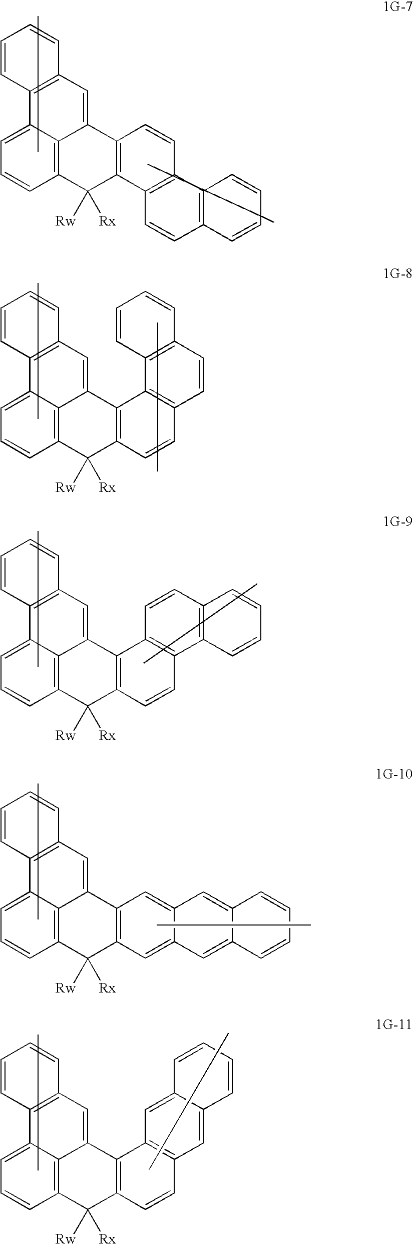

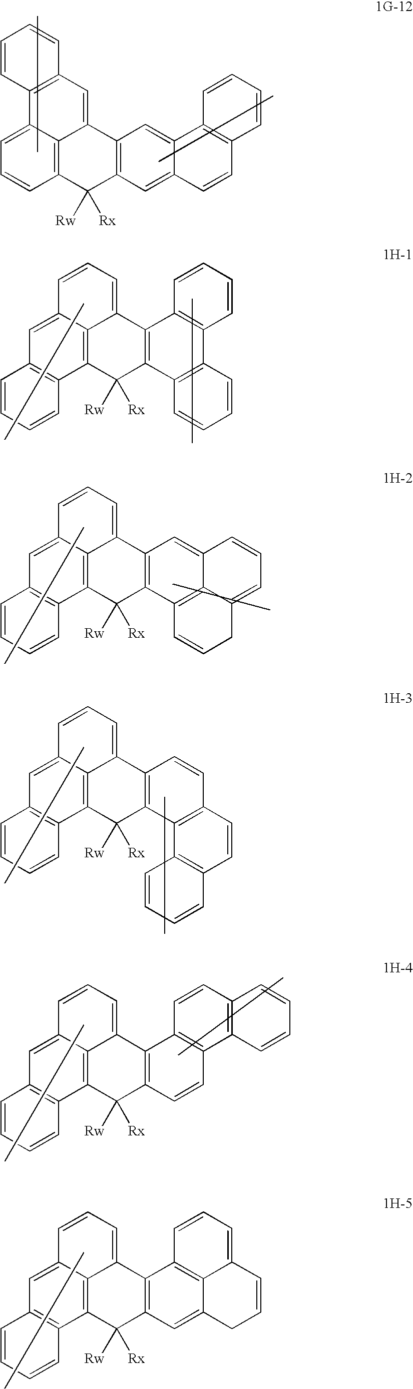

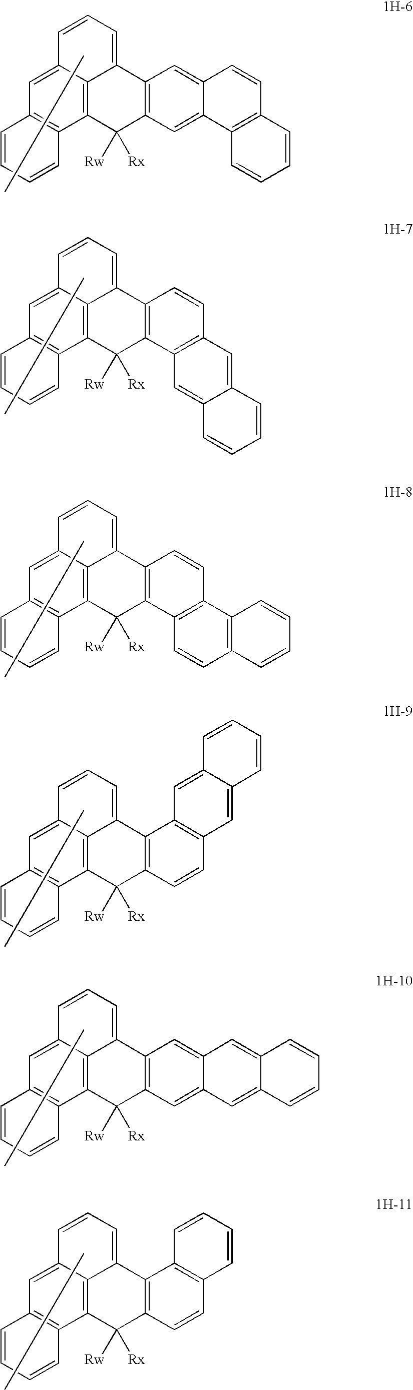

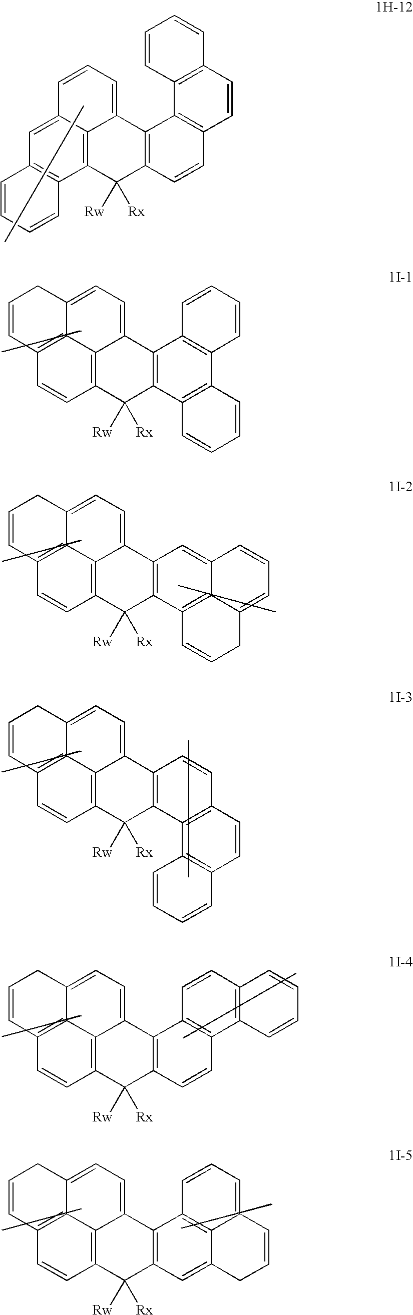

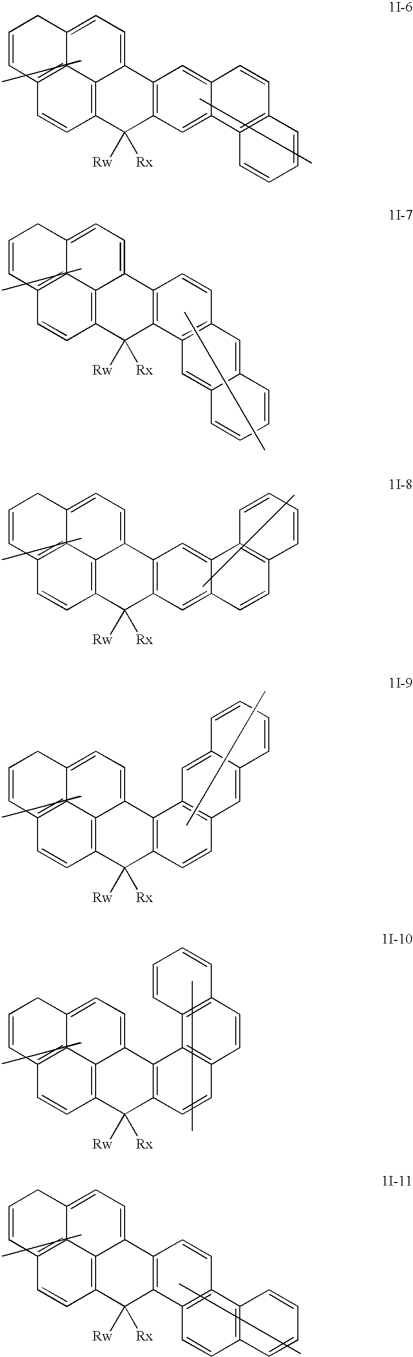

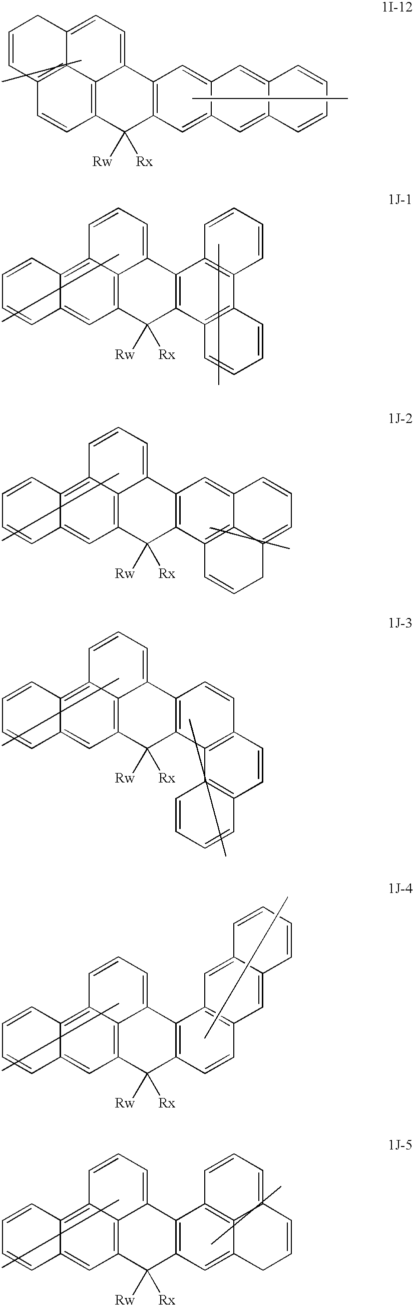

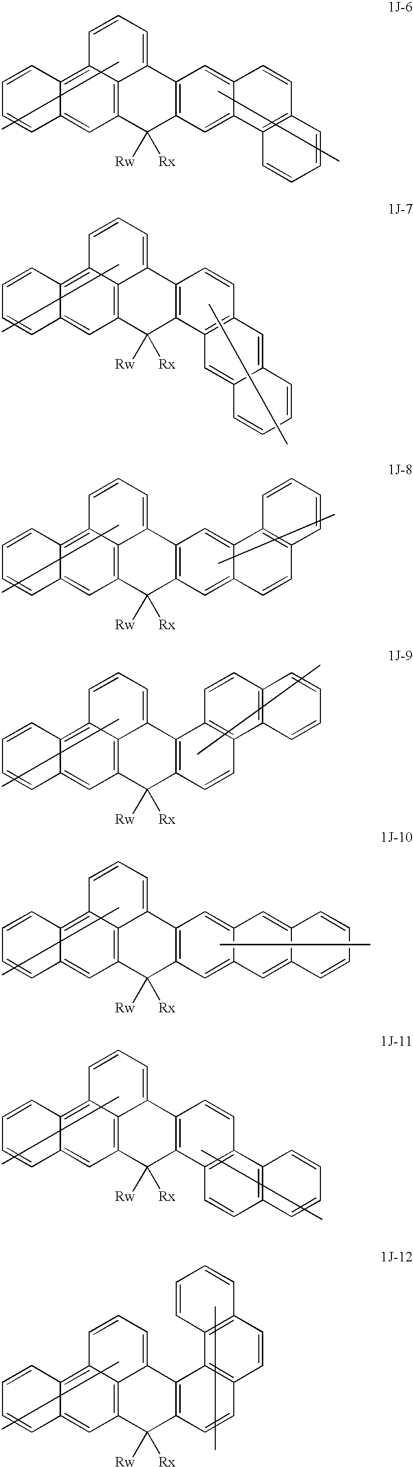

Examples of the structures of the formulae (2) and (3) in which a substituent of a ring B and a substituent of a ring C are mutually connected with each other to form a ring include those of the following formulae (1K-1) to (1K-3), (1L-1) to (1L-3), and these structures having a substituent. In the following structures, bonds on aromatic hydrocarbon rings can take any position.

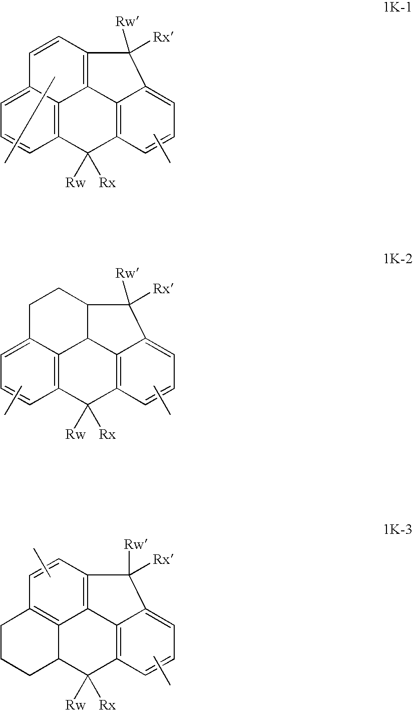

(wherein, the definitions of Rw and Rx are the same as described above. Rw′ and Rx′ represent the same substituents as for Rw and Rx.).

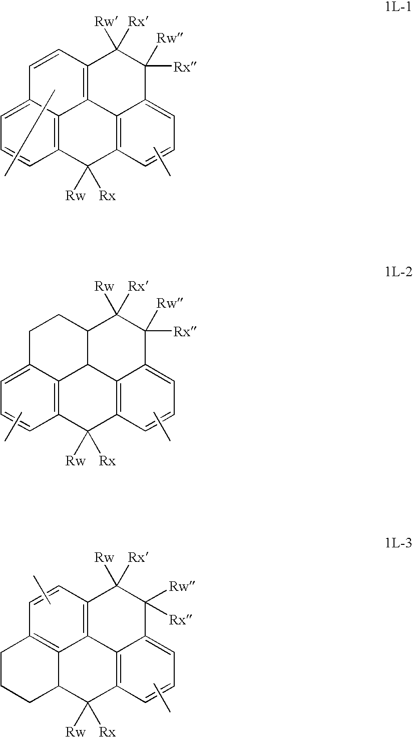

(wherein, the definitions of Rw, Rx, Rw′ and Rx′ are the same as described above. Rw″ and Rx″ represents the same substituents as for Rw and Rx.).

The structures of the formulae (2) and (3) in which Z₁ represents silicon include the following formulae (1M-1) to (1M-5), and these structures having a substituent. In the following structures, bonds on aromatic hydrocarbon rings can take any position.

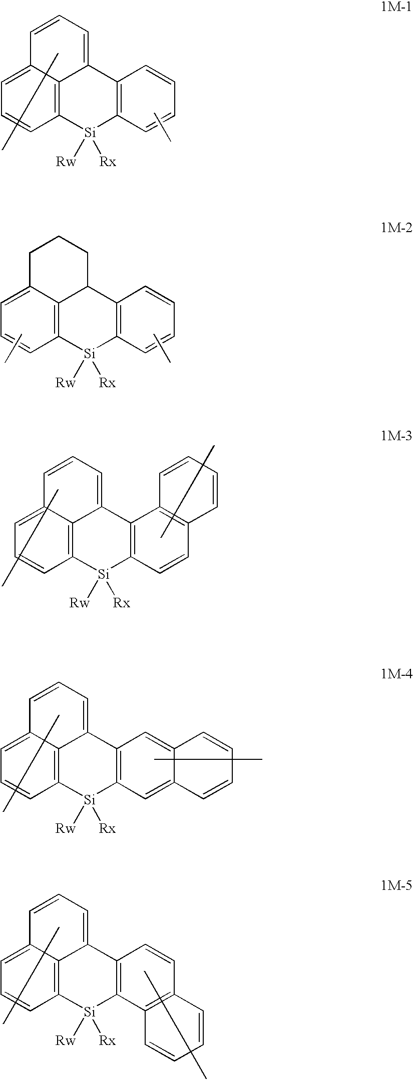

The structures of the formulae (2) and (3) in which Z₁ represents nitrogen include the following formulae (1N-1) to (1N-5), and these structures having a substituent. In the following structures, bonds on aromatic hydrocarbon rings can take any position.

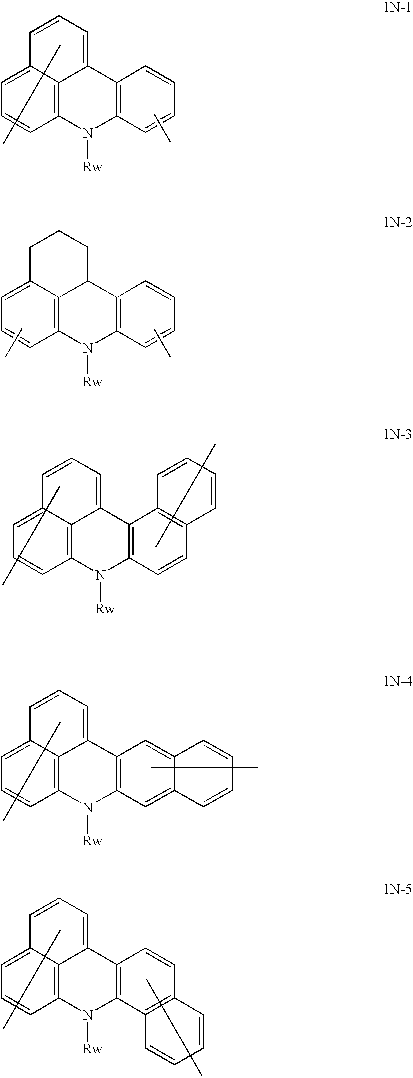

The structures of the formulae (2) and (3) in which Z₁ represents oxygen include the following formulae (1O-1) to (1O-5), and these structures having a substituent. In the following structures, bonds on aromatic hydrocarbon rings can take any position.

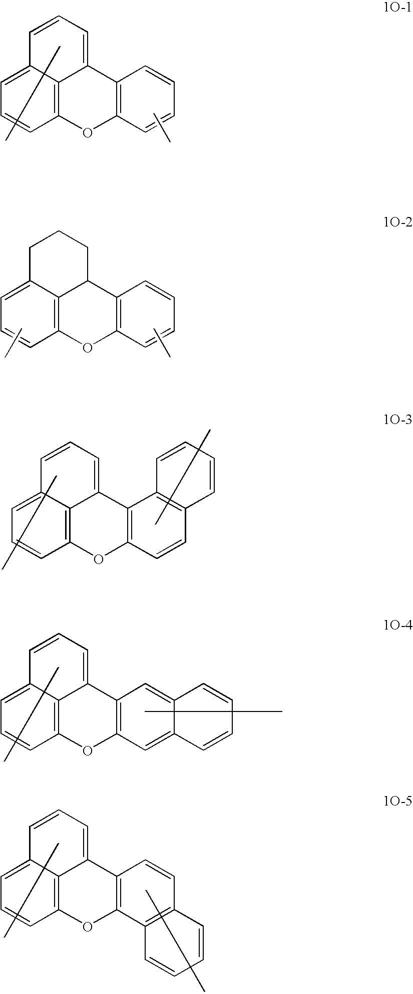

The structures of the formulae (2) and (3) in which Z₁ represents boron include the following formulae (1P-1) to (1P-5), and these structures having a substituent. In the following structures, bonds on aromatic hydrocarbon rings can take any position.

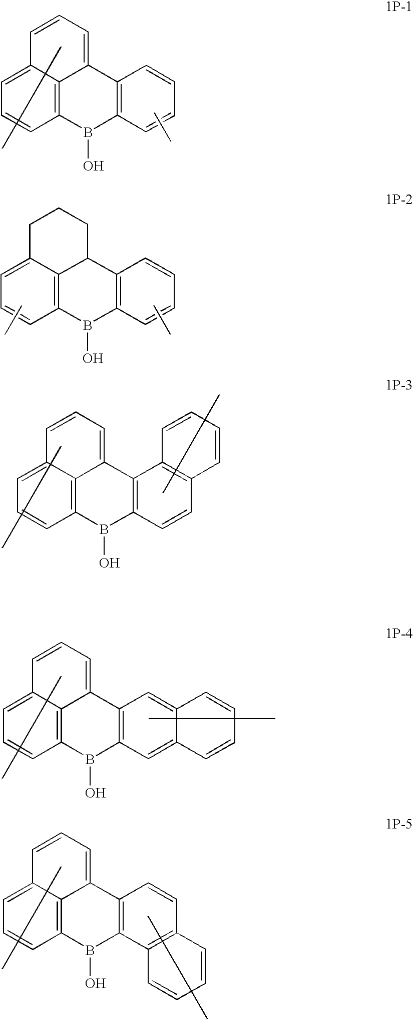

The structures of the formulae (2) and (3) in which Z₁ represents carbon include the following formulae (1Q-1) to (1Q-5), and these structures having a substituent. In the following structures, bonds on aromatic hydrocarbon rings can take any position.

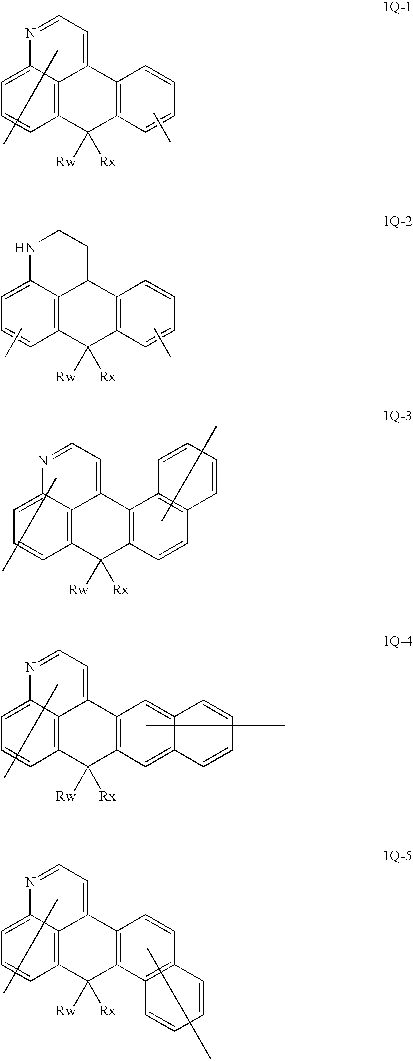

The structures of the formulae (2) and (3) in which Z₁ represents carbon and a ring B′ and a ring B″ represent a 5-membered ring include the following formulae (1R-1) to (1R-8), and these structures having a substituent. In the following structures, bonds on aromatic hydrocarbon rings can take any position.

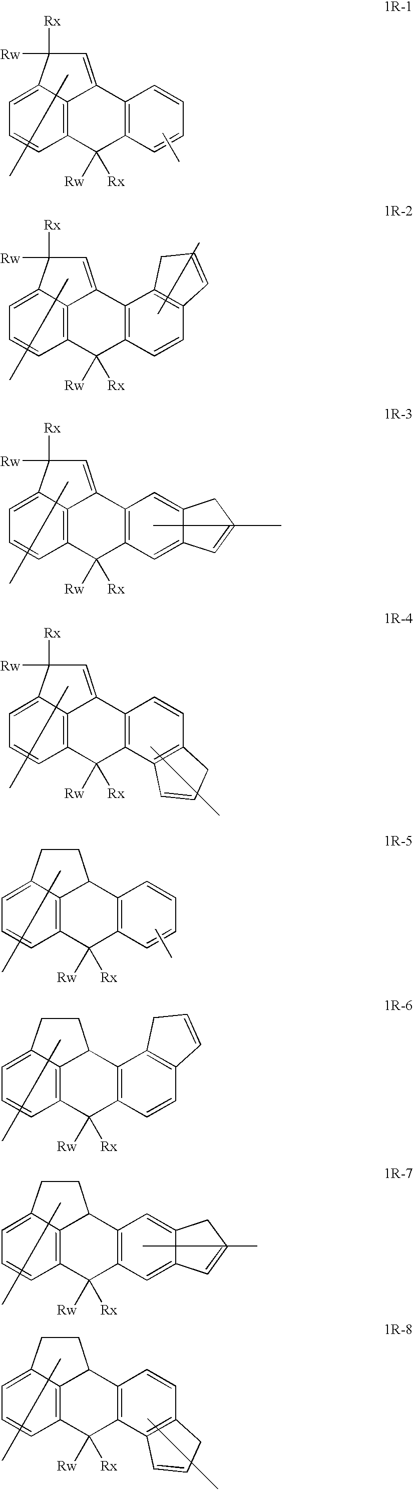

The structures of the formulae (2) and (3) in which Z₁ represents carbon and a ring B′ and a ring B″ represent a thiophene ring include the following formulae (1S-1) to (1S-3), and these structures having a substituent. In the following structures, bonds on aromatic hydrocarbon rings can take any position.

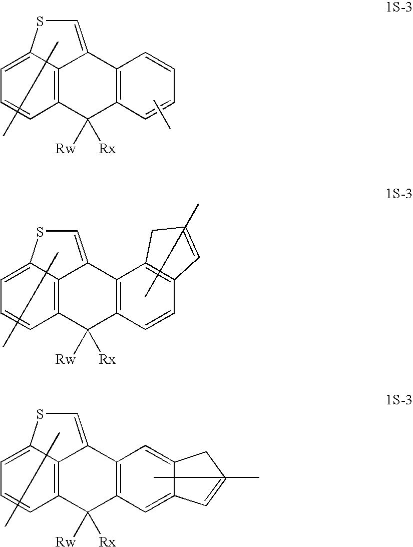

The structures of the formulae (2) and (3) in which Z₁ represents carbon and a ring B′ and a ring B″ represent a furan ring include the following formulae (1T-1) to (1T-3), and these structures having a substituent. In the following structures, bonds on aromatic hydrocarbon rings can take any position.

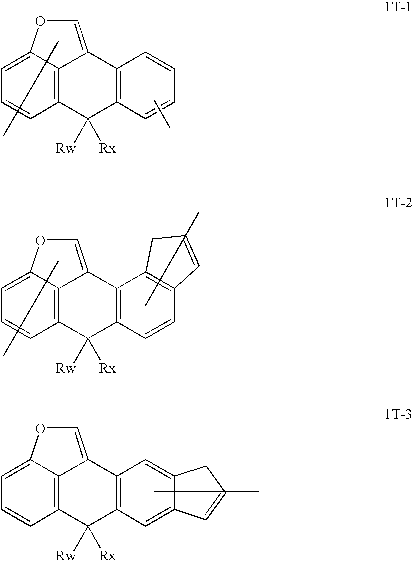

Additionally, the structures of the formulae (2) and (3) in which Z₁ represents carbon include the following formulae (1U-1) to (1U-3), and these structures having a substituent. In the following structures, bonds on aromatic hydrocarbon rings can take any position.

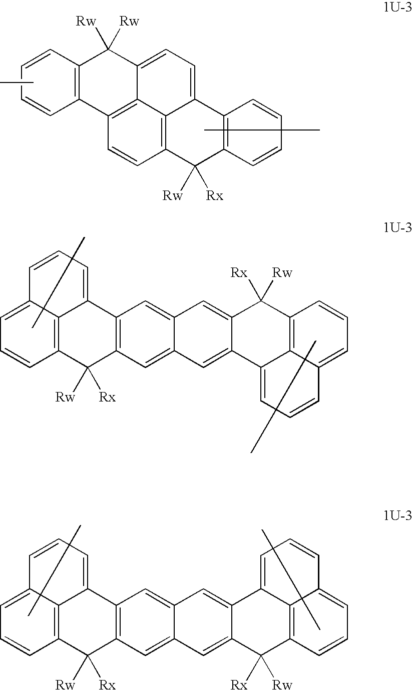

From the standpoint of stability of a compound, it is preferable that all of Z₁ to Z₃ represent a carbon atom in the structures of the formulae (1), (2) and (3).

In the structure of the formula (1), it is preferable from the standpoints of heat stability, fluorescence intensity and the like that all of a ring A, ring B and ring C represent an aromatic hydrocarbon ring, it is more preferable that they are selected from a benzene ring, naphthalene ring and anthracene ring, and it is further most preferable that all of a ring A, ring B and ring C represent a benzene ring.

In the structures of the formulae (2) and (3), it is more preferable that a ring A′, ring B′, ring C′, ring A″, ring B″ and ring C″ represent simultaneously a conjugated polymer compound having a benzene ring, and it is further preferable from the standpoint of electron injection property that one bond is present on the ring B and one bond is present on the ring C, or one bond is present on the ring A and one bond is present on the ring C.

Among others, conjugated polymer compounds containing a repeating unit of the following formulae (6) and (7) are preferable.

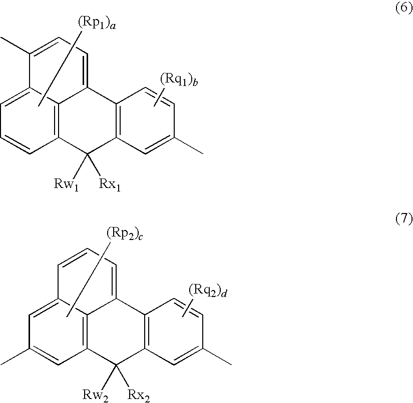

(wherein, Rₚ₁, R_{q1}, Rₚ₂, R_{q2}, R_{w1}, Rₓ₁, R_{w2} and Rₓ₂ represent each independently a substituent. a and c represent an integer of 0 to 5, and b and d represent an integer of 0 to 3. Rₚ₁ and R_{q1}, Rₚ₂ and R_{q2}, R_{w1} and Rₓ₁, and R_{w2} and Rₓ₂ each may be mutually connected to form a ring.).

It is preferable, in the formulae (6) and (7), that the carbon number of at least one of R_{w1} and Rₓ₁, and at least one of R_{w2} and Rₓ₂ is 2 or more.

In the formulae (6) and (7), Rₚ₁, R_{q1}, Rₚ₂, R_{q2}, Rₓ₁, R_{w2} and Rₓ₂ represent preferably an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, nitro group, amide group, acid imide group, monovalent heterocyclic group, carboxyl group, substituted carboxyl group and cyano group, further preferably an aryl group and arylalkyl group. As the aryl group, more specific examples include aryl groups having a carbon number of usually about from 6 to 60 such as a phenyl group, C₁ to C₁₂ alkoxyphenyl groups (C₁ to C₁₂ shows that the carbon number is 1 to 12, being applicable also in the following descriptions), C₁ to C₁₂ alkylphenyl groups, 1-naphtyl group, 2-naphtyl group, 1-anthracenyl group, 2-anthracenyl group, 9-anthracenyl group, pentafluorophenyl group and the like.

As the arylalkyl group, more specific examples are arylalkyl groups having a carbon number of usually about from 7 to 60, preferably a carbon number of about from 7 to 48 such as phenyl-C₁ to C₁₂ alkyl groups, C₁ to C₁₂ alkoxyphenyl-C₁ to C₁₂ alkyl groups, C₁ to C₁₂ alkylphenyl-C₁ to C₁₂ alkyl groups, 1-naphthyl-C₁ to C₁₂ alkyl groups, 2-naphthyl-C₁ to C₁₂ alkyl groups and the like.

From the standpoint of easiness of synthesis, conjugated polymer compounds containing a repeating unit of the above-described formula (6) are preferable.

When an aromatic ring, non-aromatic ring or Z₁ in the formula (1) has a substituent, it is preferable from the standpoints of solubility in organic solvents, device properties, easiness of synthesis, and the like that the substituent is selected from an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group, nitro group and cyano group. A hydrogen atom contained in these substituents may be substituted by a fluorine atom.

The alkyl group may be linear, branched or cyclic, the carbon number is usually about from 1 to 20, preferably 3 to 20, and examples thereof include a methyl group, ethyl group, propyl group, i-propyl group, n-butyl group, i-butyl group, t-butyl group, pentyl group, isoamyl group, hexyl group, cyclohexyl group, heptyl group, octyl group, 2-ethylhexyl group, nonyl group, decyl group, 3,7-dimethyloctyl group, lauryl group, trifluoromethyl group, pentafluoroethyl group, perfluorobutyl group, perfluorohexyl group, perfluorooctyl group and the like, and for balance between heat resistance and standpoints such as solubility in organic solvents, device properties, easiness of synthesis and the like, preferable are a pentyl group, isoamyl group, hexyl group, octyl group, 2-ethylhexyl group, decyl group and 3,7-dimethyloctyl group.

The alkoxy group may be linear, branched or cyclic, the carbon number is usually about from 1 to 20, preferably 3 to 20, and examples thereof include a methoxy group, ethoxy group, propyloxy group, i-propyloxy group, butoxy group, i-butoxy group, t-butoxy group, pentyloxy group, hexyloxy group, cyclohexyloxy group, heptyloxy group, octyloxy group, 2-ethylhexyloxy group, nonyloxy group, decyloxy group, 3,7-dimethyloctyloxy group, lauryloxy group, trifluoromethoxy group, pentafluoroethoxy group, perfluorobutoxy group, perfluorohexyl group, perfluorooctyl group, methoxymethyloxy group, 2-methoxyethyloxy group and the like, and for balance between heat resistance and standpoints such as solubility in organic solvents, device properties, easiness of synthesis and the like, preferable are a pentyloxy group, hexyloxy group, octyloxy group, 2-ethylhexyloxy group, decyloxy group and 3,7-dimethyloctyloxy group.

The alkylthio group may be linear, branched or cyclic, the carbon number is usually about from 1 to 20, preferably 3 to 20, and examples thereof include a methylthio group, ethylthio group, propylthio group, i-propylthio group, butylthio group, 1-butylthio group, t-butylthio group, pentylthio group, hexylthio group, cyclohexylthio group, heptylthio group, octylthio group, 2-ethylhexylthio group, nonylthio group, decylthio group, 3,7-dimethyloctylthio group, laurylthio group, trifluoromethylthio group and the like, and for balance between heat resistance and properties such as solubility in organic solvents, device properties, easiness of synthesis and the like, preferable are a pentylthio group, hexylthio group, octylthio group, 2-ethylhexylthio group, decylthio group and 3,7-dimethyloctylthio group.

The aryl group is an atomic group obtained by removing one hydrogen atom from an aromatic hydrocarbon, and includes also those having a condensed ring, and those in which independent two or more benzene rings or condensed rings are connected directly or via a group such as vinylene and the like. The aryl group has a carbon number of usually about from 6 to 60, preferably 7 to 48, and examples thereof include a phenyl group, C₁ to C₁₂ alkoxyphenyl groups (C₁ to C₁₂ shows that the carbon number is 1 to 12, being applicable also in the following descriptions), C₁ to C₁₂ alkylphenyl groups, 1-naphtyl group, 2-naphtyl group, 1-anthracenyl group, 2-anthracenyl group, 9-anthracenyl group, pentafluorophenyl group and the like, and from the standpoints of solubility in organic solvents, device properties, easiness of synthesis and the like, preferable are C₁ to C₁₂ alkoxyphenyl groups and C₁ to C₁₂ alkylphenyl groups. Examples of the C₁ to C₁₂ alkoxy include methoxy, ethoxy, propyloxy, i-propyloxy, butoxy, i-butoxy, t-butoxy, pentyloxy, hexyloxy, cyclohexyloxy, heptyloxy, octyloxy, 2-ethylhexyloxy, nonyloxy, decyloxy, 3,7-dimethyloctyloxy, lauryloxy and the like.

Examples of the C₁ to C₁₂ alkylphenyl group include a methylphenyl group, ethylphenyl group, dimethylphenyl group, propylphenyl group, mesityl group, methylethylphenyl group, i-propylphenyl group, butylphenyl group, i-butylphenyl group, t-butylphenyl group, pentylphenyl group, isoamylphenyl group, hexylphenyl group, heptylphenyl group, octylphenyl group, nonylphenyl group, decylphenyl group, dodecylphenyl group and the like.

The aryloxy group has a carbon number of usually about from 6 to 60, preferably 7 to 48, and examples thereof include a phenoxy group, C₁ to C₁₂ alkoxyphenoxy groups, C₁ to C₁₂ alkylphenoxy groups, 1-naphtyloxy group, 2-naphtyloxy group, pentafluorophenyloxy group and the like, and from the standpoints of solubility in organic solvents, device properties, easiness of synthesis and the like, preferable are C₁ to C₁₂ alkoxyphenoxy groups and C₁ to C₁₂ alkylphenoxy groups.

Examples of the C₁ to C₁₂ alkoxy include methoxy, ethoxy, propyloxy, i-propyloxy, butoxy, i-butoxy, t-butoxy, pentyloxy, hexyloxy, cyclohexyloxy, heptyloxy, octyloxy, 2-ethylhexyloxy, nonyloxy, decyloxy, 3,7-dimethyloctyloxy, lauryloxy and the like.

Examples of the C₁ to C₁₂ alkylphenoxy group include a methylphenoxy group, ethylphenoxy group, dimethylphenoxy group, propylphenoxy group, 1,3,5-trimethylphenoxy group, methylethylphenoxy group, i-propylphenoxy group, butylphenoxy group, i-butylphenoxy group, t-butylphenoxy group, pentylphenoxy group, isoamylphenoxy group, hexylphenoxy group, heptylphenoxy group, octylphenoxy group, nonylphenoxy group, decylphenoxy group, dodecylphenoxy group and the like.

The arylthio group has a carbon number of usually about from 3 to 60, and examples thereof include a phenylthio group, C₁ to C₁₂ alkoxyphenylthio groups, C₁ to C₁₂ alkylphenylthio groups, 1-naphtylthio group, 2-naphtylthio group, pentafluorophenylthio group and the like, and from the standpoints of solubility in organic solvents, device properties, easiness of synthesis and the like, preferable are C₁ to C₁₂ alkoxyphenylthio groups and C₁ to C₁₂ alkylphenylthio groups.

The arylalkyl group has a carbon number of usually about from 7 to 60, preferably 7 to 48, and examples thereof include phenyl-C₁ to C₁₂ alkyl groups, C₁ to C₁₂ alkoxyphenyl-C₁ to C₁₂ alkyl groups, C₁ to C₁₂ alkylphenyl-C₁ to C₁₂ alkyl groups, 1-naphthyl-C₁ to C₁₂ alkyl groups, 2-naphthyl-C₁ to C₁₂ alkyl groups and the like, and from the standpoints of solubility in organic solvents, device properties, easiness of synthesis and the like, preferable are C₁ to C₁₂ alkoxyphenyl-C₁ to C₁₂ alkyl groups and C₁ to C₁₂ alkylphenyl-C₁ to C₁₂ alkyl groups.

The arylalkoxy group has a carbon number of usually about from 7 to 60, preferably 7 to 48, and examples thereof include phenyl-C₁ to C₁₂ alkoxy groups such as a phenylmethoxy group, phenylethoxy group, phenylbutoxy group, phenylpentyloxy group, phenylhexyloxy group, phenylheptyloxy group, phenyloctyloxy group and the like, C₁ to C₁₂ alkoxyphenyl-C₁ to C₁₂ alkoxy groups, C₁ to C₁₂ alkylphenyl-C₁ to C₁₂ alkoxy groups, 1-naphthyl-C₁ to C₁₂ alkoxy groups, 2-naphthyl-C₁ to C₁₂ alkoxy groups and the like, and from the standpoints of solubility in organic solvent, device properties, easiness of synthesis and the like, preferable are C₁ to C₁₂ alkoxyphenyl-C₁ to C₁₂ alkoxy groups and C₁ to C₁₂ alkylphenyl-C₁ to C₁₂ alkoxy groups.

The arylalkylthio group has a carbon number of usually about from 7 to 60, preferably 7 to 48, and examples thereof include phenyl-C₁ to C₁₂ alkylthio groups, C₁ to C₁₂ alkoxyphenyl-C₁ to C₁₂ alkylthio groups, C₁ to C₁₂ alkylphenyl-C₁ to C₁₂ alkylthio groups, 1-naphthyl-C₁ to C₁₂ alkylthio groups, 2-naphthyl-C₁ to C₁₂ alkylthio groups and the like, and from the standpoints of solubility in organic solvents, device properties, easiness of synthesis and the like, preferable are C₁ to C₁₂ alkoxyphenyl-C₁ to C₁₂ alkylthio groups and C₁ to C₁₂ alkylphenyl-C₁ to C₁₂ alkylthio groups.

The arylalkenyl group has a carbon number of usually about from 8 to 60, and examples thereof include phenyl-C₂ to C₁₂ alkenyl groups, C₁ to C₁₂ alkoxyphenyl-C₂ to C₁₂ alkenyl groups, C₁ to C₁₂ alkylphenyl-C₂ to C₁₂ alkenyl groups, 1-naphthyl-C₂ to C₁₂ alkenyl groups, 2-naphthyl-C₂ to C₁₂ alkenyl groups and the like, and from the standpoints of solubility in organic solvents, device properties, easiness of synthesis and the like, preferable are C₁ to C₁₂ alkoxyphenyl-C₂ to C₁₂ alkenyl groups and C₁ to C₁₂ alkylphenyl-C₂ to C₁₂ alkenyl groups.

The arylalkynyl group has a carbon number of usually about from 8 to 60, and examples thereof include phenyl-C₂ to C₁₂ alkynyl groups, C₁ to C₁₂ alkoxyphenyl-C₂ to C₁₂ alkynyl groups, C₁ to C₁₂ alkylphenyl-C₂ to C₁₂ alkynyl groups, 1-naphthyl-C₂ to C₁₂ alkynyl groups, 2-naphthyl-C₂ to C₁₂ alkynyl groups and the like, and from the standpoints of solubility in organic solvents, device properties, easiness of synthesis and the like, preferable are C₁ to C₁₂ alkoxyphenyl-C₂ to C₁₂ alkynyl groups and C₁ to C₁₂ alkylphenyl-C₂ to C₁₂ alkynyl groups.

The substituted amino group includes amino groups substituted with one or two groups selected from an alkyl group, aryl group, arylalkyl group or monovalent heterocyclic group, and the alkyl group, aryl group, arylalkyl group or monovalent heterocyclic group optionally has a substituent. The carbon number of the substituted amino group is usually about from 1 to 60, preferably 2 to 48 not including the carbon number of the substituent.

Examples include a methylamino group, dimethylamino group, ethylamino group, diethylamino group, propylamino group, dipropylamino group, i-propylamino group, diisopropylamino group, butylamino group, i-butylamino group, t-butylamino group, pentylamino group, hexylamino group, cyclohexylamino group, heptylamino group, octylamino group, 2-ethylhexylamino group, nonylamino group, decylamino group, 3,7-dimethyloctylamino group, laurylamino group, cyclopentylamino group, dicyclopentylamino group, cyclohexylamino group, dicyclohexylamino group, pyrrolidyl group, piperidyl group, ditrifluoromethylamino group, phenylamino group, diphenylamino group, C₁ to C₁₂ alkoxyphenylamino group, di(C₁ to C₁₂ alkoxyphenyl)amino group, di(C₁ to C₁₂ alkylphenyl)amino group, 1-naphthylamino group, 2-naphthylamino group, pentafluorophenylamino group, pyridylamino group, pyridazinylamino group, pyrimidylamino group, pyrazylamino group, triazylamino group, phenyl-C₁ to C₁₂ alkylamino group, C₁ to C₁₂ alkoxyphenyl-C₁ to C₁₂ alkylamino group, C₁ to C₁₂ alkylphenyl-C₁ to C₁₂ alkylamino group, di(C₁ to C₁₂ alkoxyphenyl-C₁ to C₁₂ alkyl)amino group, di (C₁ to C₁₂ alkylphenyl-C₁ to C₁₂ alkyl)amino group, 1-naphthyl-C₁ to C₁₂ alkylamino group, 2-naphthyl-C₁ to C₁₂ alkylamino group and the like.

The substituted silyl group includes silyl groups substituted with one, two or three groups selected from an alkyl group, aryl group, arylalkyl group or monovalent heterocyclic group. The carbon number of the substituted silyl group is usually about from 1 to 60, preferably 3 to 48. The alkyl group, aryl group, arylalkyl group or monovalent heterocyclic group optionally has a substituent.

Examples include a trimethylsilyl group, triethylsilyl group, tripropylsilyl group, tri-i-propylsilyl group, dimethyl-i-propylsilyl group, diethyl-i-propylsilyl group, t-butylsilyldimethylsilyl group, pentyldimethylsilyl group, hexyldimethylsilyl group, heptyldimethylsilyl group, octyldimethylsilyl group, 2-ethylhexyl-dimethylsilyl group, nonyldimethylsilyl group, decyldimethylsilyl group, 3,7-dimethyloctyl-dimethylsilyl group, lauryldimethylsilyl group, phenyl-C₁ to C₁₂ alkylsilyl group, C₁ to C₁₂ alkoxyphenyl-C₁ to C₁₂ alkylsilyl group, C₁ to C₁₂ alkylphenyl-C₁ to C₁₂ alkylsilyl group, 1-naphthyl-C₁ to C₁₂ alkylsilyl group, 2-naphthyl-C₁ to C₁₂ alkylsilyl group, phenyl-C₁ to C₁₂ alkyldimethylsilyl group, triphenylsilyl group, tri-p-xylylsilyl group, tribenzylsilyl group, diphenylmethylsilyl group, t-butyldiphenylsilyl group, dimethylphenylsilyl group and the like.

Examples of the halogen atom include a fluorine atom, chlorine atom, bromine atom and iodine atom.

The acyl group has a carbon number of usually about from 2 to 20, preferably 2 to 18, and examples thereof include an acetyl group, propionyl group, butyryl group, isobutyryl group, pivaloyl group, benzoyl group, trifluoroacetyl group, pentafluorobenzoyl group and the like.

The acyloxy group has a carbon number of usually about from 2 to 20, preferably 2 to 18, and examples thereof include an acetoxy group, propionyloxy group, butyryloxy group, isobutyryloxy group, pivaloyloxy group, benzoyloxy group, trifluoroacetyloxy group, pentafluorobenzoyloxy group and the like.

The imine residue includes residues obtained by removing one hydrogen atom from imine compounds (meaning organic compounds having —N═C— in the molecule. Examples thereof include aldimines, ketimines, and compounds obtained by substituting a hydrogen atom on N of these compounds by an alkyl group and the like), and has a carbon number of usually about from 2 to 20, preferably 2 to 18. Specific examples include groups of the following structural formulae, and the like.

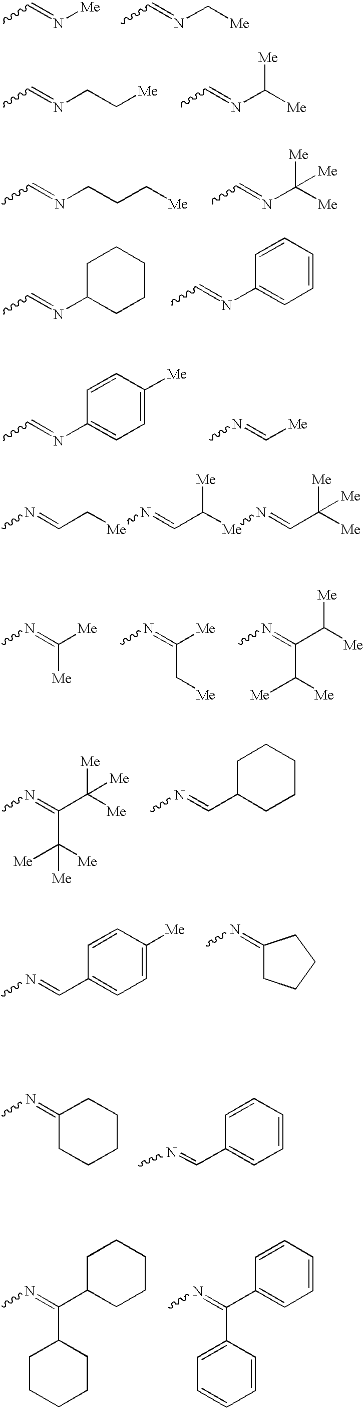

The amide group has a carbon number of usually about from 2 to 20, preferably 2 to 18, and examples thereof include a formamide group, acetamide group, propioamide group, butyroamide group, benzamide group, trifluoroacetamide group, pentafluorobenzamide group, diformamide group, diacetamide group, dipropioamide group, dibutyroamide group, dibenzamide group, ditrifluoroacetamide group, dipentafluorobenzamide group and the like.

The acid imide group includes residues obtained by removing from an acid imide a hydrogen atom bonded to its nitrogen atom, and the carbon number is about from 4 to 20, and specific examples include the following groups and the like.

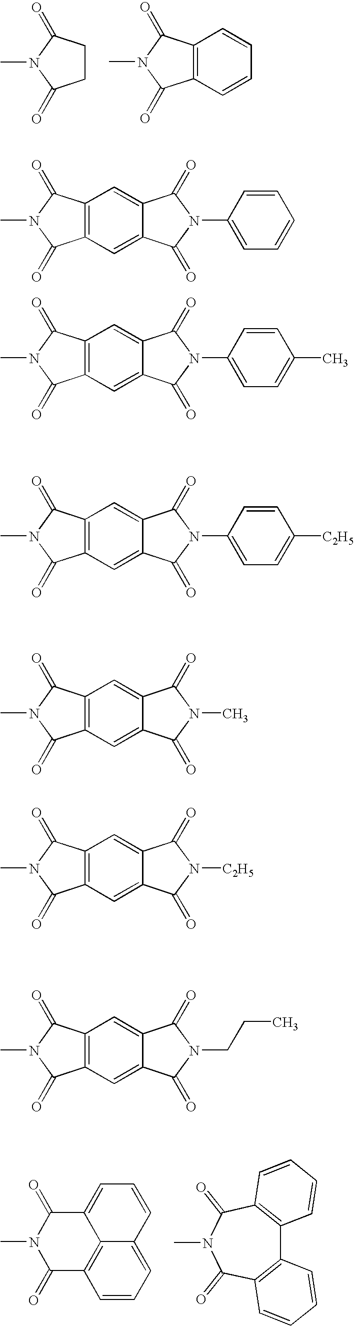

The monovalent heterocyclic group means an atomic group remaining after removing one hydrogen atom from a heterocyclic compound, and the carbon number is usually about from 4 to 60, preferably 4 to 20. The carbon number of a heterocyclic group does not include the carbon number of a substituent. Here, the heterocyclic compound refers to organic compounds having a cyclic structure in which elements constituting the cyclic structure include not only a carbon atom, but also a heteroatom such as oxygen, sulfur, nitrogen, phosphorus, boron, silicon and the like contained in the ring. Examples include a thienyl group, C₁ to C₁₂ alkylthienyl group, pyrrolyl group, furyl group, pyridyl group, C₁ to C₁₂ alkylpyridyl group, piperidyl group, quinolyl group, isoquinolyl group and the like, and preferable are a thienyl group, C₁ to C₁₂ alkylthienyl group, pyridyl group and C₁ to C₁₂ alkylpyridyl group.

The substituted carboxyl group includes carboxyl groups substituted with an alkyl group, aryl group, arylalkyl group or monovalent heterocyclic group, and the carbon number is usually about from 2 to 60, preferably 2 to 48, and examples thereof include a methoxycarbonyl group, ethoxycarbonyl group, propoxycarbonyl group, i-propoxycarbonyl group, butoxycarbonyl group, i-butoxycarbonyl group, t-butoxycarbonyl group, pentyloxycarbonyl group, hexyloxycarbonyl group, cyclohexyloxycarbonyl group, heptyloxycarbonyl group, octyloxycarbonyl group, 2-ethylhexyloxycarbonyl group, nonyloxycarbonyl group, decyloxycarbonyl group, 3,7-dimethyloctyloxycarbonyl group, dodecyloxycarbonyl group, trifluoromethoxycarbonyl group, pentafluoroethoxycarbonyl group, perfluorobutoxycarbonyl group, perfluorohexyloxycarbonyl group, perfluorooctyloxycarbonyl group, phenoxycarbonyl group, naphthoxycarbonyl group, pyridyloxycarbonyl group, and the like. The alkyl group, aryl group, arylalkyl group or monovalent heterocyclic group optionally has a substituent. The carbon number of the substituted carboxyl group does not include the carbon number of the substituent.

In a conjugated polymer compound of the present invention, it is preferable that Rw, Rx, Rw1, Rx1, Rw2 and Rx2 represent each independently an aryl group or arylalkyl group, and it is more preferable that Rw and Rx represent the same aryl group, that Rw1 and Rx1 represent the same aryl group, and that Rw2 and Rx2 represent the same aryl group, from the standpoint of electron injection prorperty.

Here, the definitions and examples of the aryl group and arylalkyl group are the same as described above.

The aryl group includes a phenyl group, 2-methylphenyl group, 3-methylphenyl group, 4-methylphenyl group, 2,6-dimethylphenyl group, 3,5-dimethylphenyl group, 2,4,6-trimethylphenyl group, 2-ethylphenyl group, 3-ethylphenyl group, 4-ethylphenyl group, 2,6-diethylphenyl group, 3,5-diethylphenyl group, 2-propylphenyl group, 3-propylphenyl group, 4-propylphenyl group, 2,6-dipropylphenyl group, 3,5-dipropylphenyl group, 2,4,6-tripropylphenyl group, 2-isopropylphenyl group, 3-isopropylphenyl group, 4-isopropylphenyl group, 2,6-diisopropylphenyl group, 3,5-diisopropylphenyl group, 2,4,6-triisopropylphenyl group, 2-butylphenyl group, 3-butylphenyl group, 4-butylphenyl group, 2,6-butylphenyl group, 3,5-butylphenyl group, 2,4,6-butylphenyl group, 2-t-butylphenyl group, 3-t-butylphenyl group, 4-t-butylphenyl group, 2,6-di-t-butylphenyl group, 3,5-di-t-butylphenyl group, 2,4,6-tri-t-butylphenyl group and the like, the carbon number thereof is preferably about from 6 to 20, and a phenyl group is further preferable, from the standpoints of electron injection property, solubility, device properties and the like.

From the standpoint of solubility of a compound, examples of structures of repeating units of the above-described formula (2) include the following formulae (2A-1) to (2A-3).

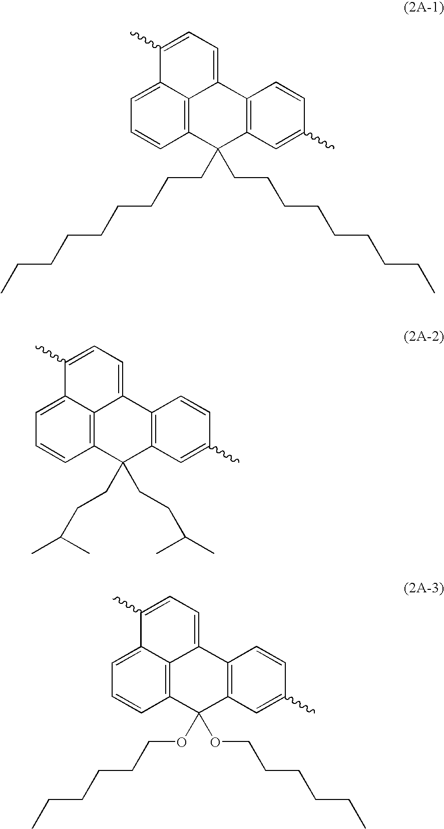

From the standpoint of solubility of a compound, examples of structures of repeating units of the above-described formula (3) include the following formulae (3A-1) to (3A-3).

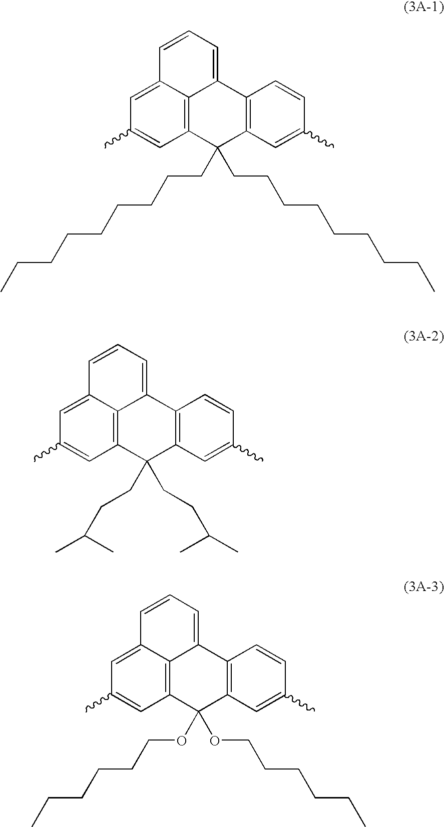

Structures of repeating units of the above-described formula (6) excellent in electron injection property include those in which Rw1 and Rx1 represent the same aryl group, and examples include the following formulae (2B-1) to (2B-4).

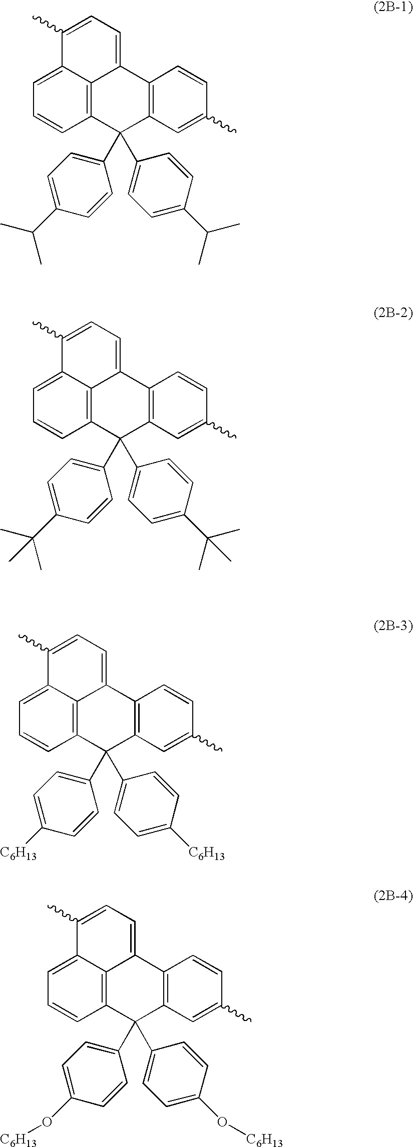

Structures of repeating units of the above-described formula (7) excellent in electron injection property include those in which Rw1 and Rx1 represent the same aryl group, and examples include the following formulae (3B-1) to (3B-4).

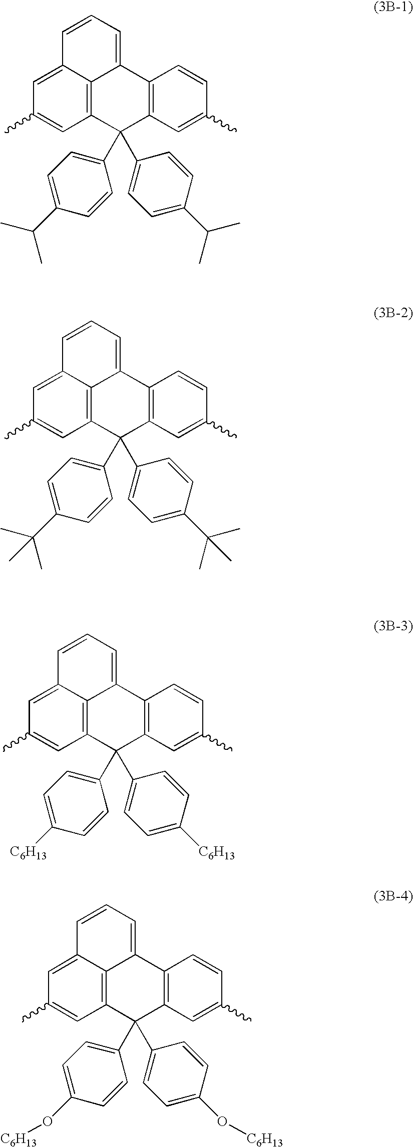

Regarding examples of repeating units of the above-described formula (2) examples of those in which Rw and Rx are mutually connected to form a ring include the following formulae (2C-1) to (2C-4).

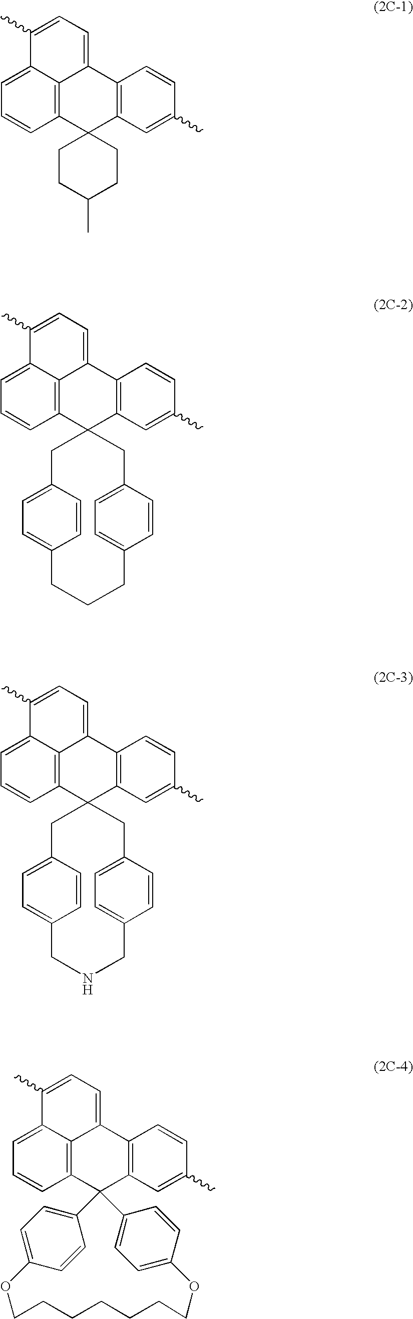

Regarding examples of repeating units of the above-described formula (3), examples of those in which Rw and Rx are mutually connected to form a ring include the following formulae (3C-1) to (3C-4).

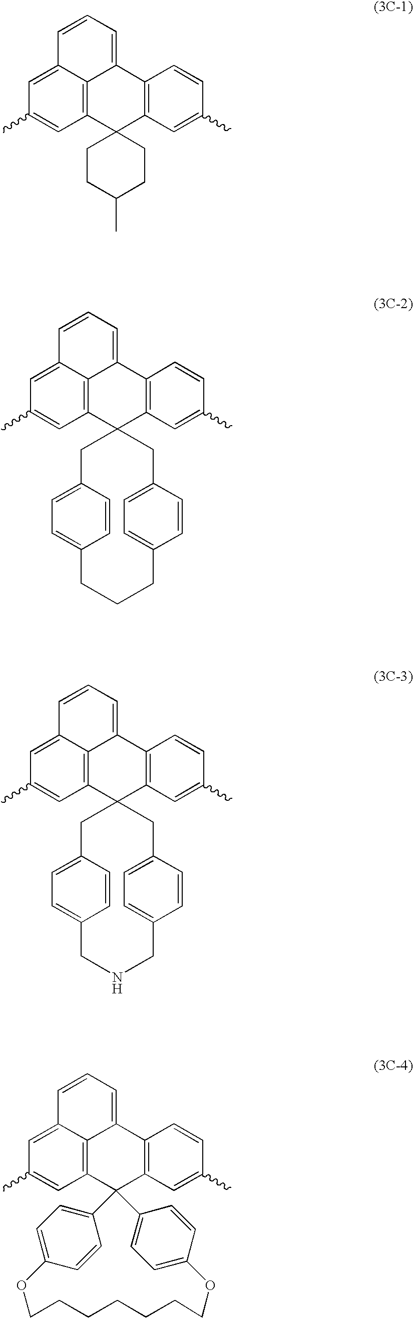

Regarding repeating units of the above-described formula (2), examples of those in which substituents of a ring B′ and a ring C′ are mutually connected to form a ring include structures of the following formulae (2D-1) to (2D-4).

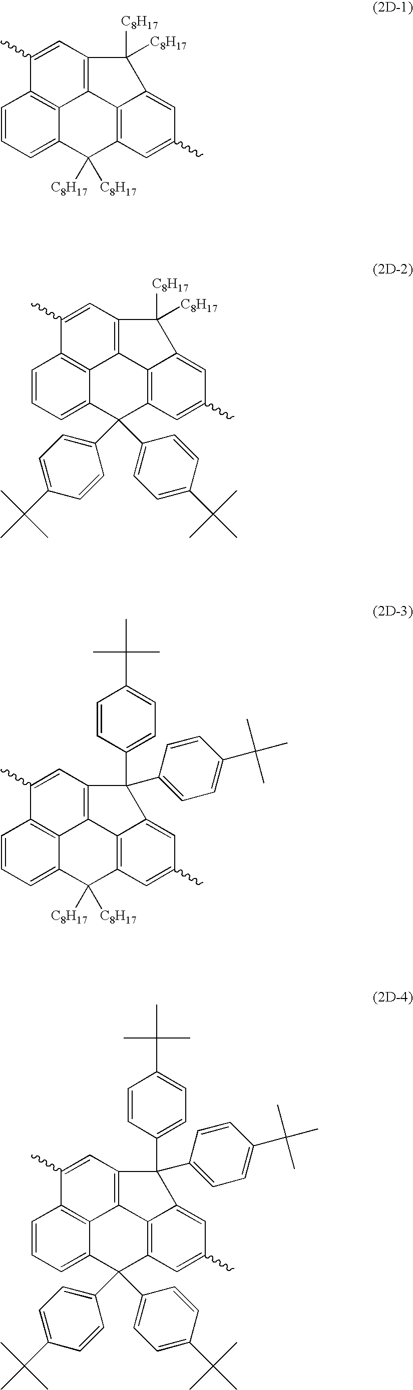

Regarding repeating units of the above-described formula (3), examples of those in which substituents of a ring B″ and a ring C″ are mutually connected to form a ring include structures of the following formulae (3D-1) to (3D-4).

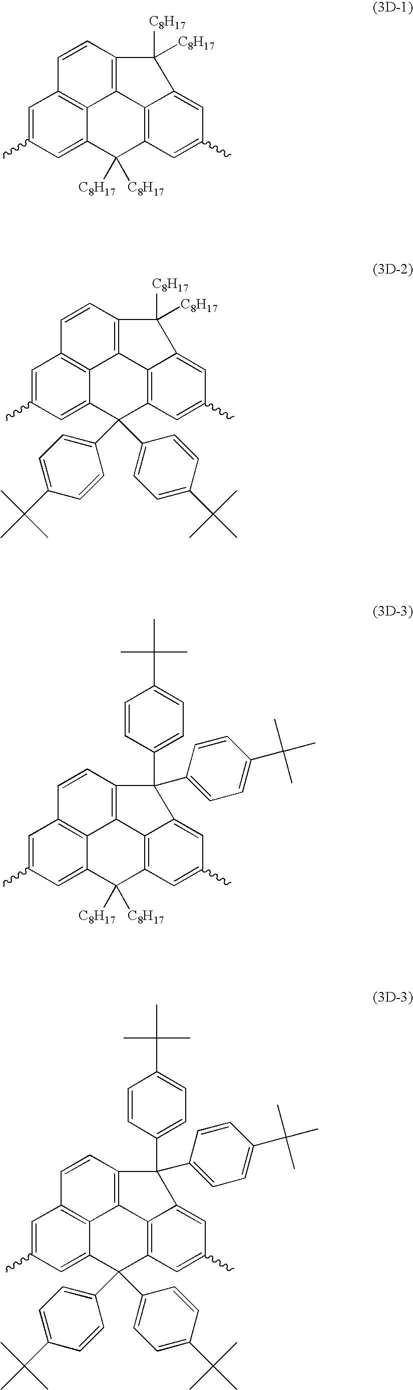

Among repeating units of the above-described formula (6), examples of those in which Rw1 and R1x represent a non-aromatic ring such as aliphatic hydrocarbon rings, non-aromatic heterocyclic rings and the like include the following units.

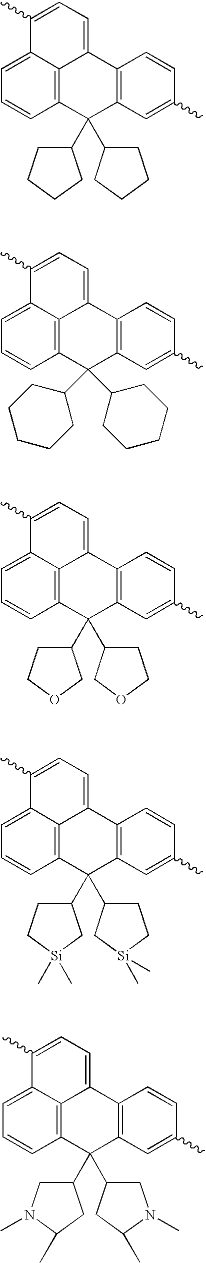

Among repeating units of the above-described formula (7), examples of those in which Rw1 and R1x represent a non-aromatic ring such as aliphatic hydrocarbon rings, non-aromatic heterocyclic rings and the like include the following units.

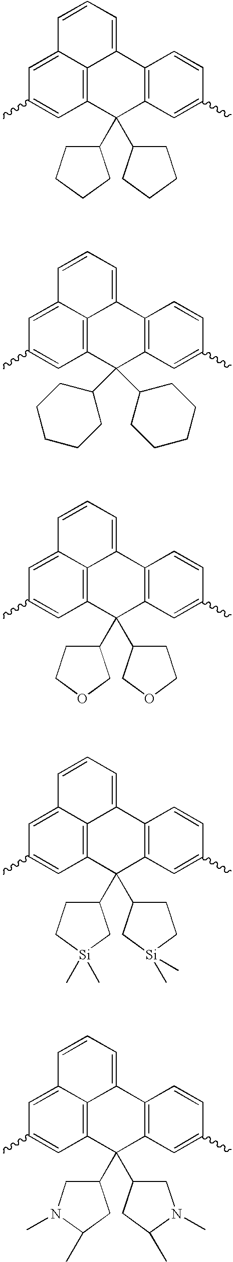

From the standpoint of heat resistance of a compound, at least one of R_{w1} and Rₓ₁, or at least one of R_{w2} and Rₓ₂ is a substituent having a carbon number of preferably 2 or more, more preferably 4 to 12.

Other preferable structures of the above-described formula (1) include structures of the following formula (4).

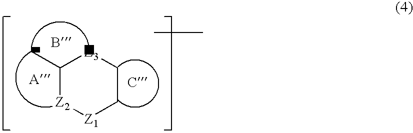

(wherein, a ring A′″ and a ring B′″ represent an aromatic ring optionally having a substituent or a non-aromatic ring optionally having a substituent, and at least one of the ring A′″ and the ring B′″ is an aromatic ring optionally having a substituent. A ring C′″ represents an aromatic ring optionally having a substituent. A bond is present on the ring A′″, the ring B′″ or the ring C′″. Z₁ to Z₃ represent the same meanings as described above.).

The definitions, examples and the like of the aromatic ring optionally having a substituent or the non-aromatic ring optionally having a substituent in the formula (4) are the same as the definitions and examples thereof in the above-described formula (1).

The structure of the formula (4) is present on the side chain or end of a conjugated polymer compound. In such cases, repeating units of the conjugated polymer compound may or may not contain a structure of the above-described formula (2) or (3).

Examples of the structure of the formula (4) include structures obtained by deleting one bond from the above-described structures (1A-1) to (1U-3) and these structures having a substituent.

Other preferable structures of the above-described formula (1) include structures of the following formula (5).

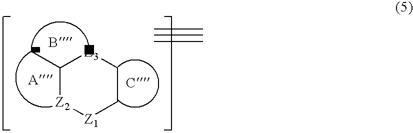

(wherein, a ring A″″ and a ring B″″ represent each independently an aromatic ring optionally having a substituent or a non-aromatic ring optionally having a substituent, and at least one of the ring A and the ring B is an aromatic ring optionally having a substituent. A ring C″″ represents an aromatic hydrocarbon ring optionally having a substituent. Three bonds are present on the ring A″″, the ring B″″ or the ring C″″, and Z₁ to Z₃ represent the same meanings as described above.).

Any one of the ring A″″, ring B″″ and ring C″″ may have a plurality of bonds.

The definitions, examples and the like of the aromatic ring optionally having a substituent or the non-aromatic ring optionally having a substituent in the formula (5) are the same as the definitions and specific examples thereof in the above-described formula (1).

In the case of inclusion of a structure of the above-described formula (5), a conjugated polymer compound usually takes a branched structure. In the case of inclusion of the above-described formula (5) as a repeating unit, the proportion of repeating units the above-described formula (5) based on all repeating units is preferably 10 mol % or less, more preferably 1 mol % or less, from the standpoints of solubility and the like.

Examples of the structure of the above-described formula (5), include structures obtained by adding one bond to any of a ring A, ring B and ring C of the above-described structures (1A-1) to (1U-3).

The conjugated polymer compound of the present invention preferably contains at least one structure other than the above-described formula (1), from the standpoint of a light emitting property.

Further, the polymer compound preferably contains at least one repeating unit having a structure other than (1).

The repeating unit having a structure other than (1) includes repeating units of the following formulae (8) to (11).

—Ar₁— (8)

—(Ar₂—X₁)ₑ—Ar₃— (9)

—Ar₄—X₂— (10)

—X₃— (11)

(wherein, Ar₁, Ar₂, Ar₃ and Ar₄ represent each independently an arylene group, divalent heterocyclic group or divalent group having a metal complex structure. X₁, X₂ and X₃ represent each independently —CR₉=═CR₁₀—, —N(R₁₁)— or —(SiR₁₂R₁₃)ₘ—. R₉ and R₁₀ represent each independently a hydrogen atom, alkyl group, aryl group, monovalent heterocyclic group, carboxyl group, substituted carboxyl group or cyano group. R₁₁, R₁₂ and R₁₃ represent each independently a hydrogen atom, alkyl group, aryl group, monovalent heterocyclic group, arylalkyl group or substituted amino group. ff represents 1 or 2. m represents an integer of 1 to 12. When there are two or more R₉s, R₁₀s, R₁₁s, R₁₂s or R₁₃s, respectively, they may be the same or different.).

Here, the arylene group is an atomic group obtained by removing two hydrogen atoms from an aromatic hydrocarbon, and includes those having a condensed ring, and those obtained by two or more independent benzene rings or condensed rings directly or via a group such as vinylene and the like. The arylene group optionally has a substituent. The substituent includes an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group and cyano group.

The carbon number of a portion excluding substituents of the arylene group is usually about from 6 to 60, preferably 6 to 20. The total carbon number including substituents of the arylene group is usually about from 6 to 100.

Examples of the arylene group include phenylene groups (for example, the following formulae 1 to 3), naphthalenediyl groups (the following formulae 4 to 13), anthracene-diyl groups (the following formulae 14 to 19), biphenyl-diyl groups (the following formulae 20 to 25), fluorene-diyl groups (the following formulae 36 to 38), terphenyl-diyl groups (the following formulae 26 to 28), condensed ring compound groups (the following formulae 29 to 35), stilbene-diyl (the following formulae A to D), distilbene-diyl (the following formulae E, F), and the like. Of them, preferable are phenylene groups, biphenylene groups, fluorene-diyl groups and stilbene-diyl groups.

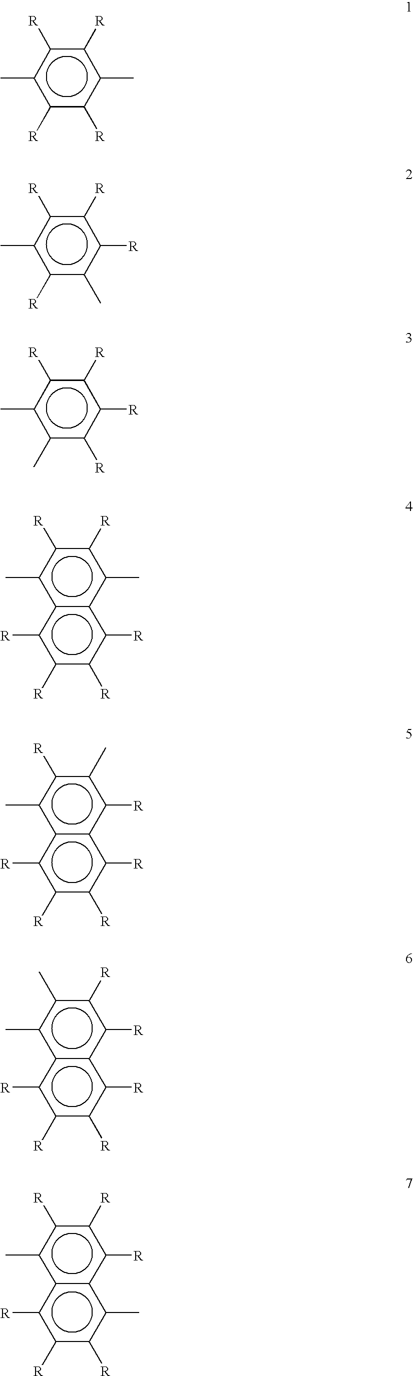

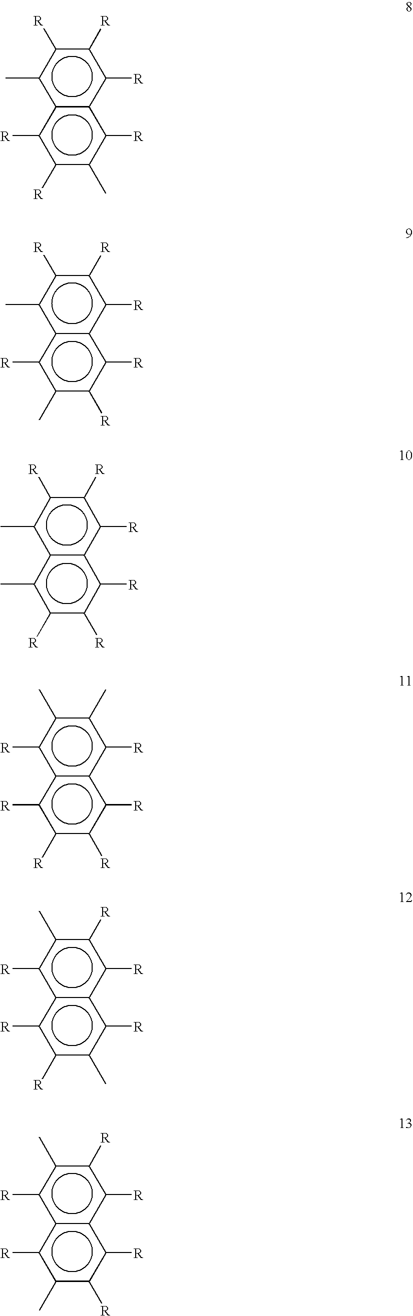

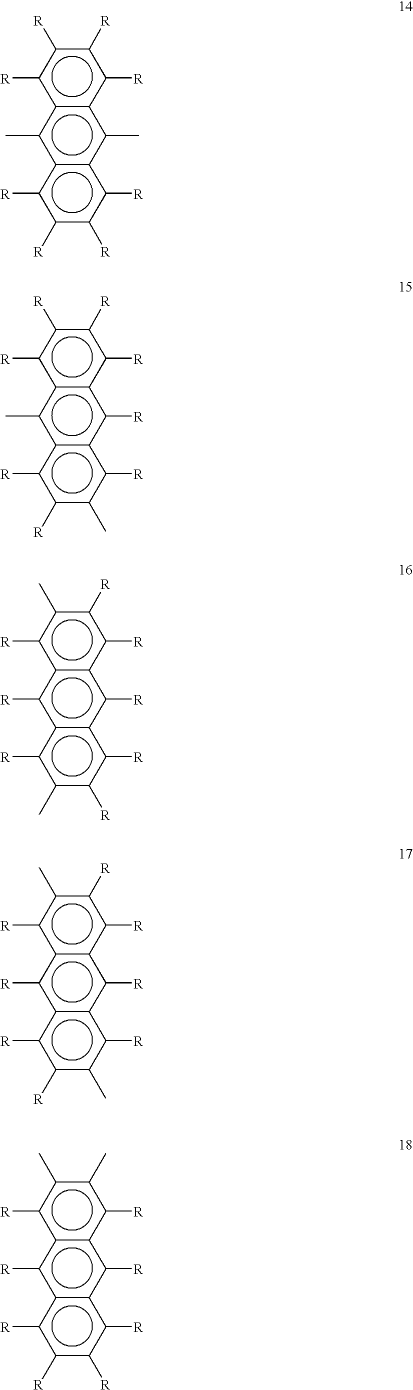

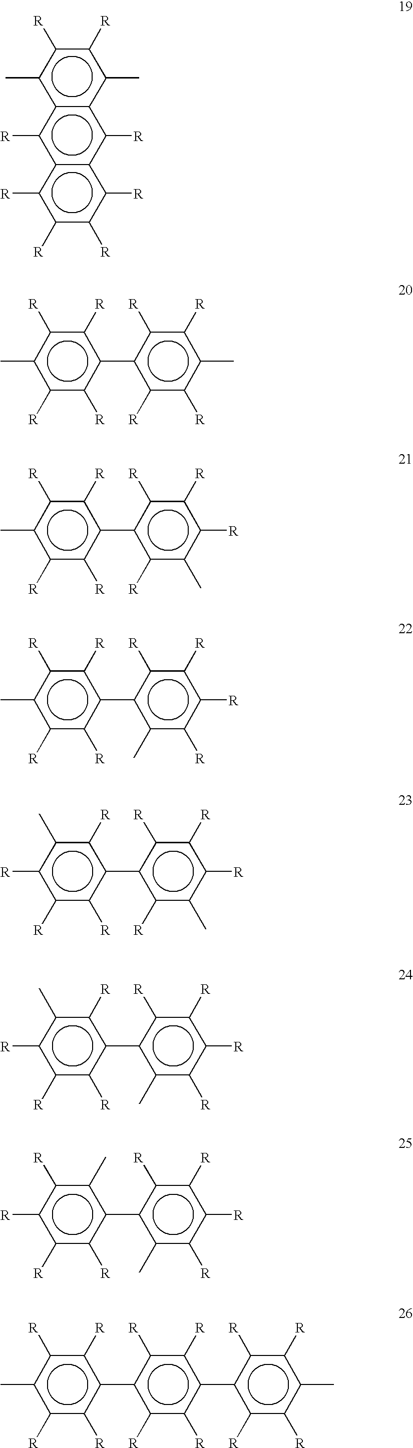

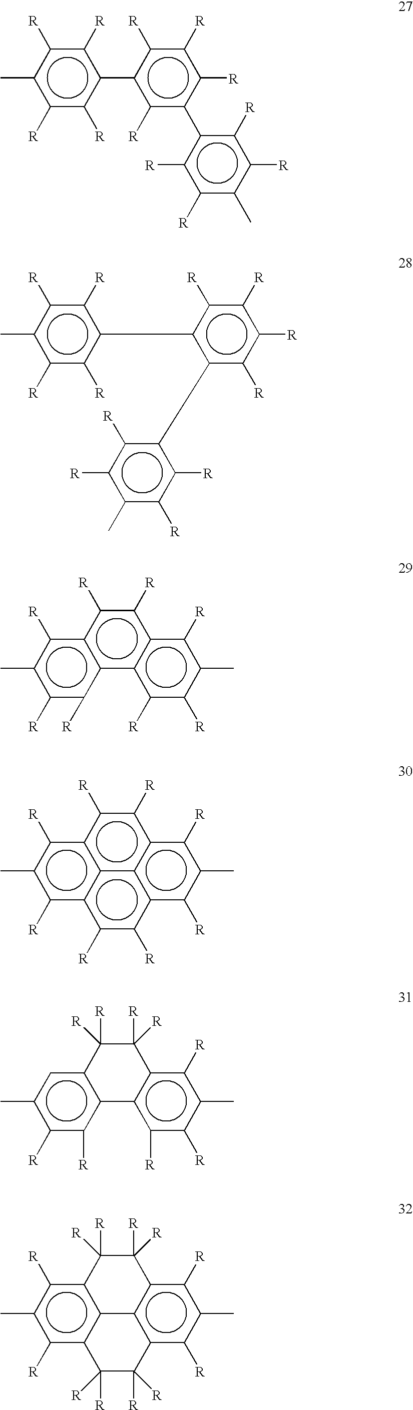

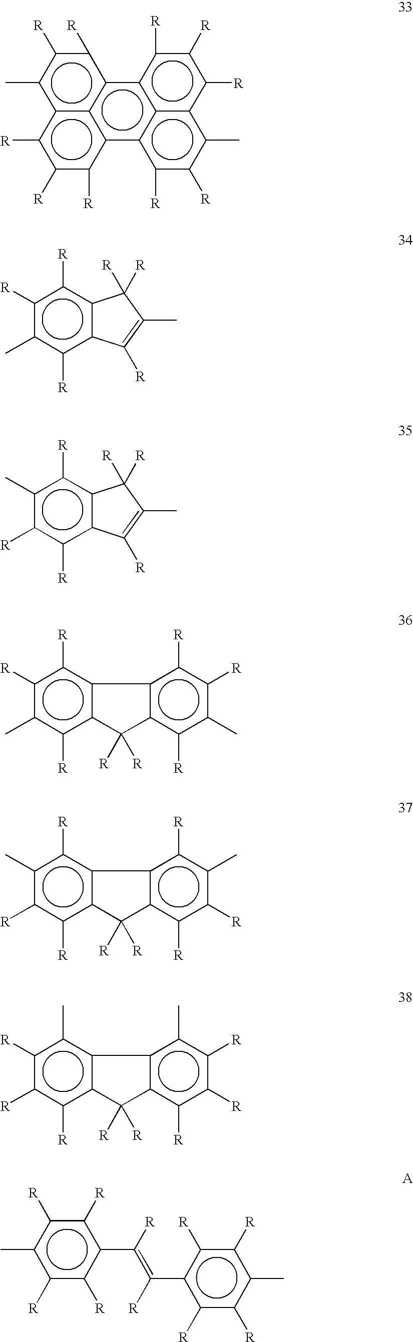

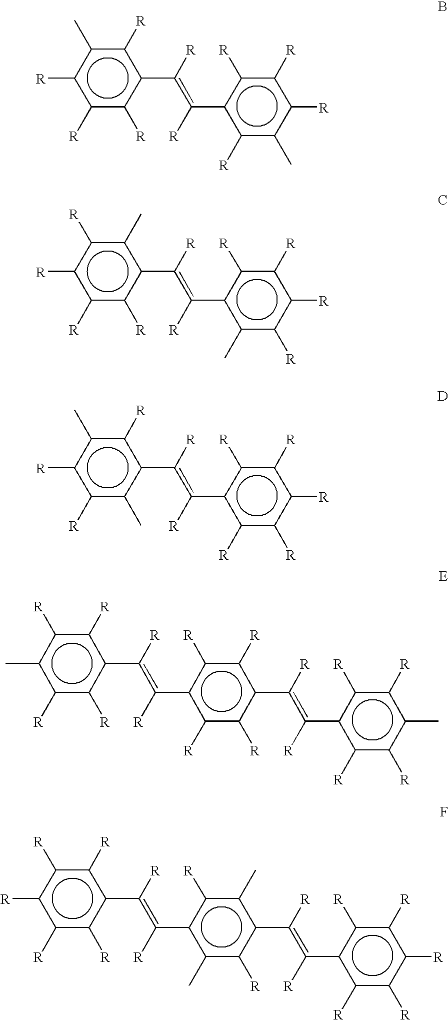

The divalent heterocyclic group for Ar₁, Ar₂, Ar₃ and Ar₄ means an atomic group remaining after removing two hydrogen atoms from a heterocyclic compound, and this group optionally has a substituent. Here, the heterocyclic compound refers to organic compounds having a cyclic structure in which elements constituting the ring include not only a carbon atom, but also a heteroatom such as oxygen, sulfur, nitrogen, phosphorus, boron, arsenic and the like contained in the ring. Of divalent heterocyclic groups, aromatic heterocyclic groups are preferable.

The substituent includes an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group and cyano group.

The carbon number of a portion excluding substituents of the divalent heterocyclic group is usually about from 3 to 60. The total carbon number including substituents of the divalent heterocyclic group is usually about from 3 to 100.

Examples of the divalent heterocyclic group include the following groups; divalent heterocyclic groups containing nitrogen as a heteroatom; pyridine-diyl group (the following formulae 38 to 44), diazaphenylene group (the following formulae 45 to 48), quinolinediyl group (the following formulae 49 to 63), quinoxalinediyl group (the following formulae 64 to 68), acridinediyl group (the following formulae 69 to 72), bipyridyldiyl group (the following formulae 73 to 75), phenanthrolinediyl group (the following formulae 76 to 78), and the like.

Groups containing oxygen, silicon, nitrogen, selenium and the like as a heteroatom and having a fluorene structure (the following formulae 79 to 93).

5-membered ring heterocyclic groups containing oxygen, silicon, nitrogen, sulfur, selenium and the like as a heteroatom (the following formulae 94 to 98) are mentioned.

5-membered ring condensed heterocyclic groups containing oxygen, silicon, nitrogen, selenium and the like as a heteroatom (the following formulae 99 to 110) are mentioned.

5-membered ring heterocyclic groups containing oxygen, silicon, nitrogen, sulfur, selenium and the like as a heteroatom, and having bonding at α-position of its heteroatom to form a dimer or oligomer (the following formulae 111 to 112) are mentioned.

5-membered ring heterocyclic groups containing oxygen, silicon, nitrogen, sulfur, selenium and the like as a heteroatom, and having bonding to a phenyl group at α-position of its heteroatom (the following formulae 113 to 119) are mentioned.

5-membered ring condensed heterocyclic groups containing oxygen, nitrogen, sulfur and the like as a heteroatom, and having substitution with a phenyl group, furyl group or thienyl group (the following formulae 120 to 125).

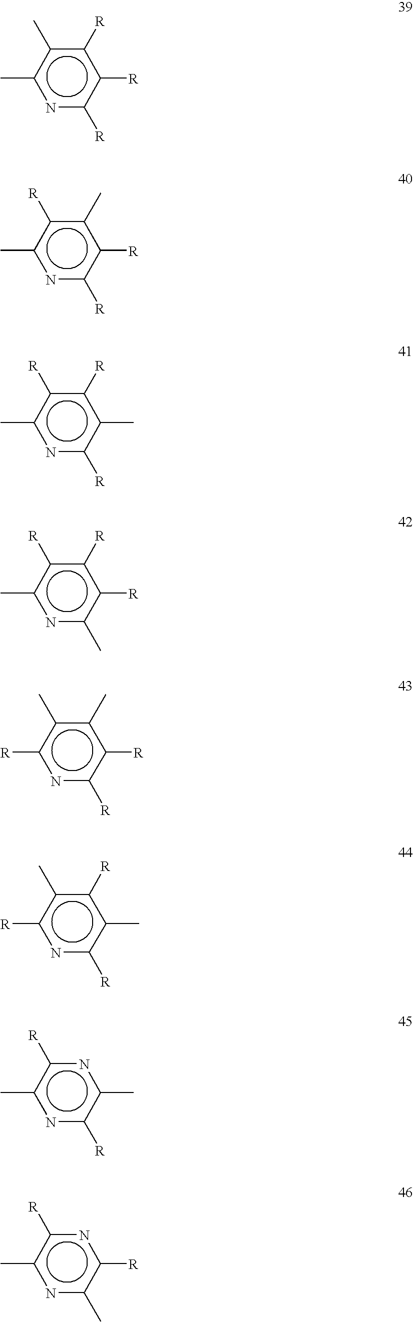

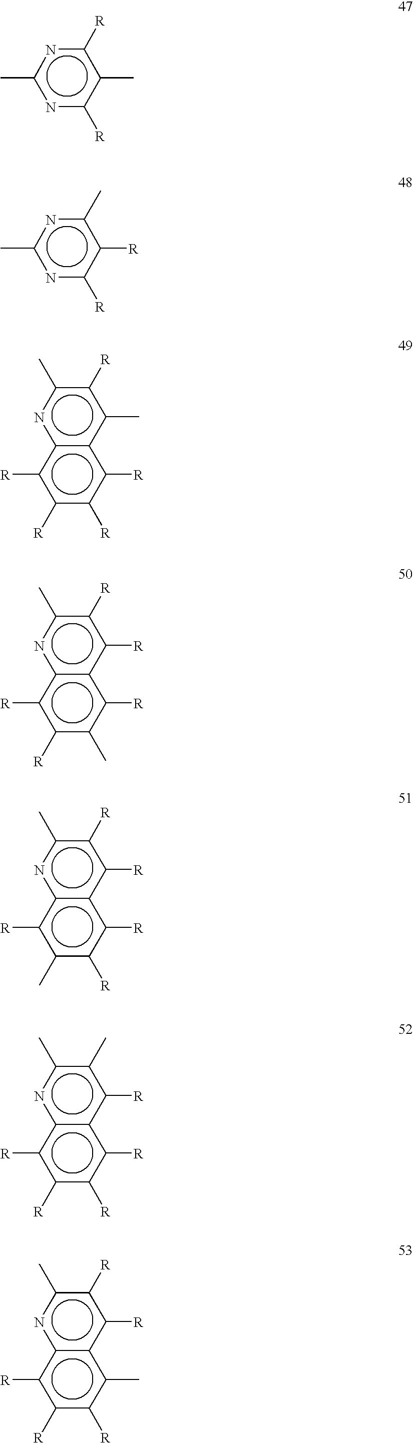

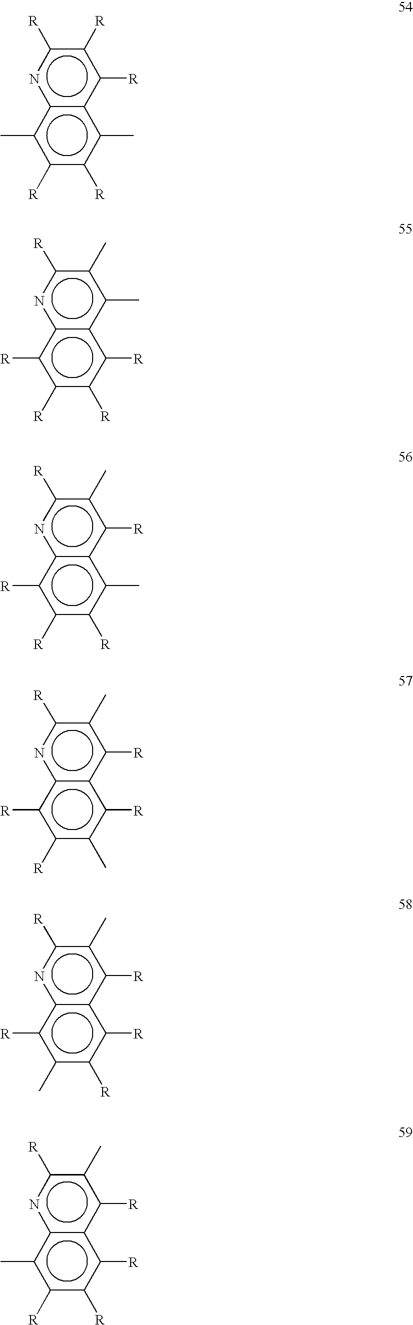

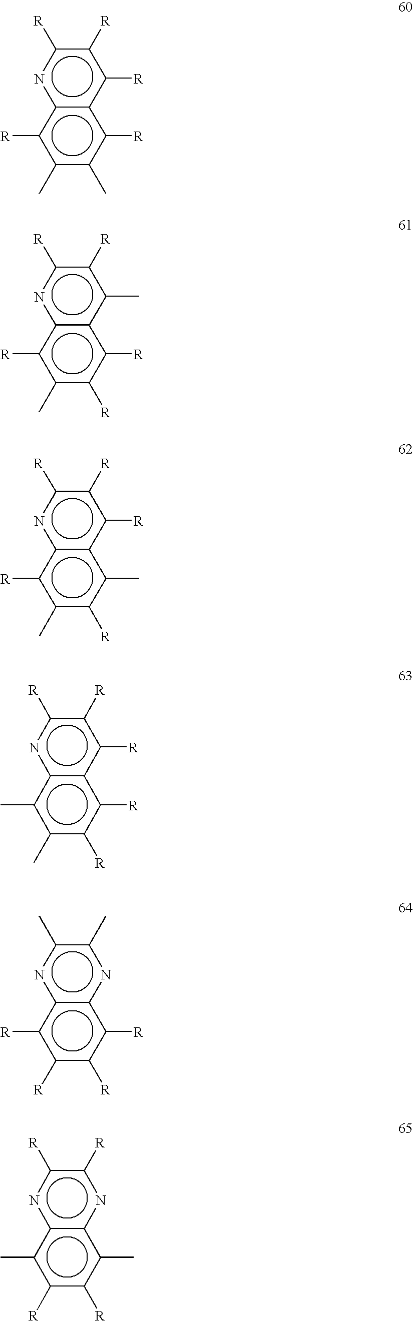

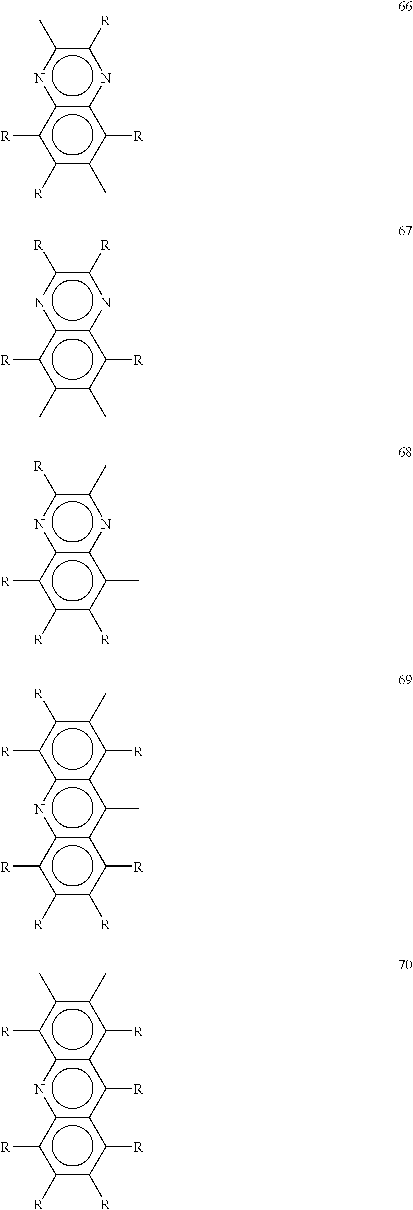

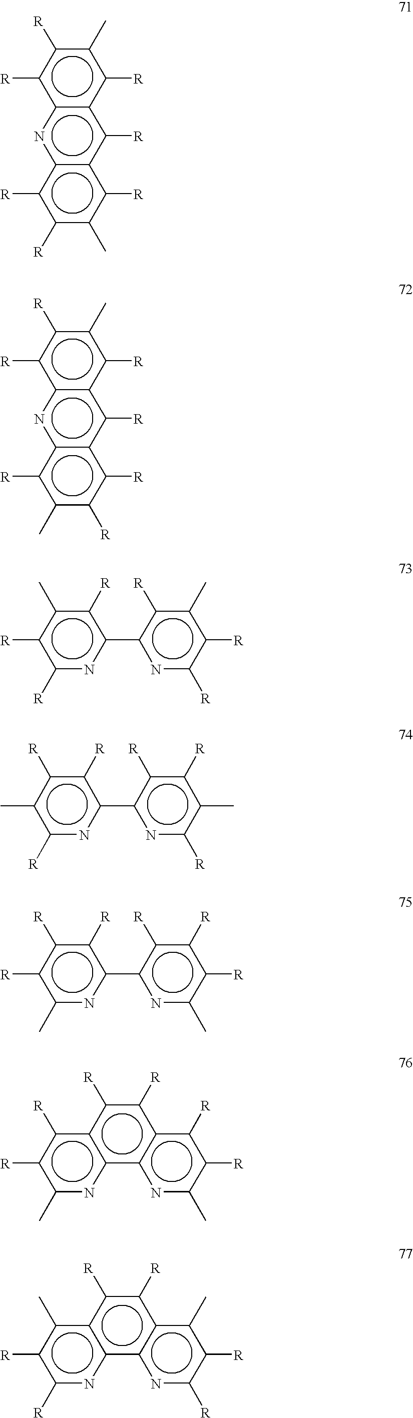

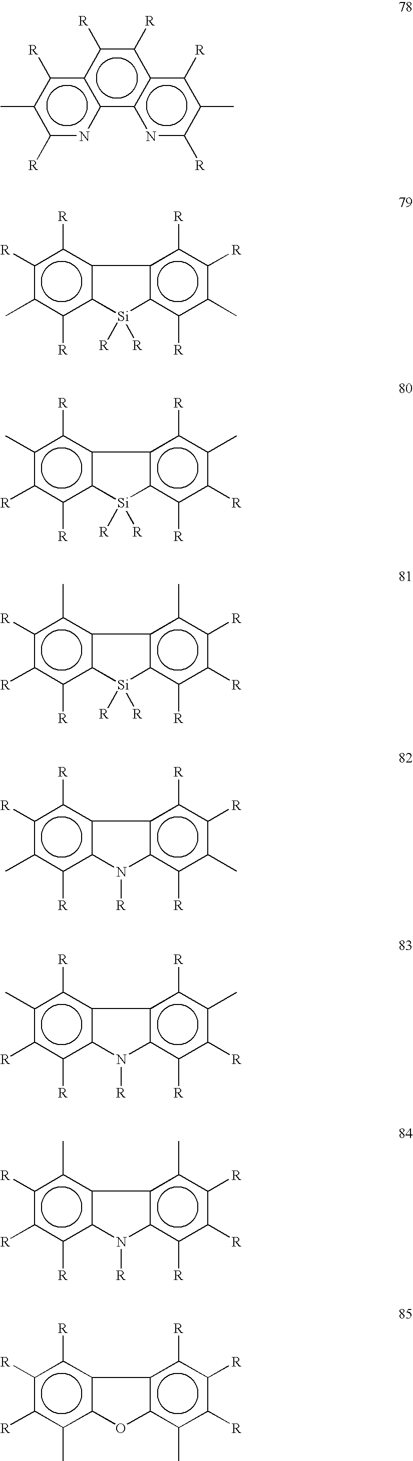

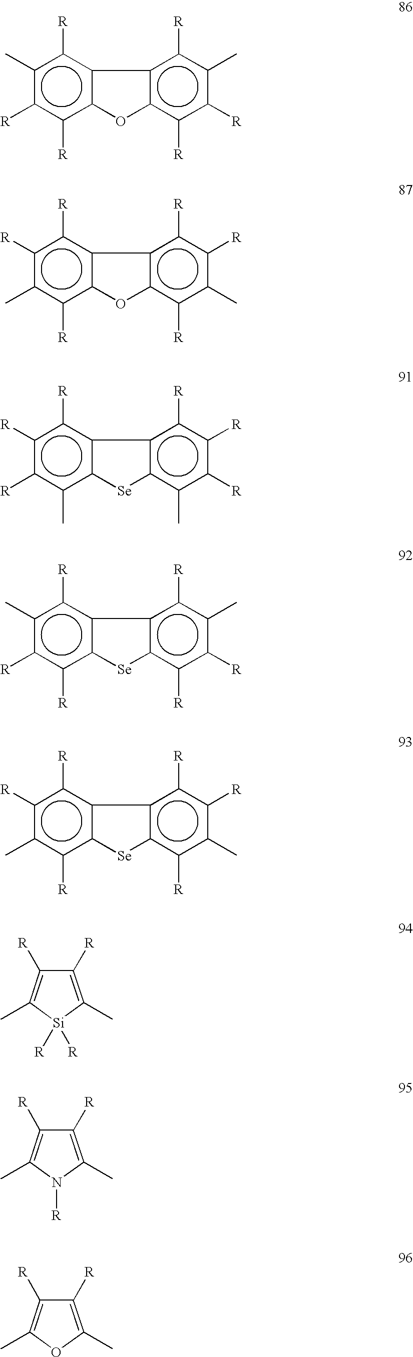

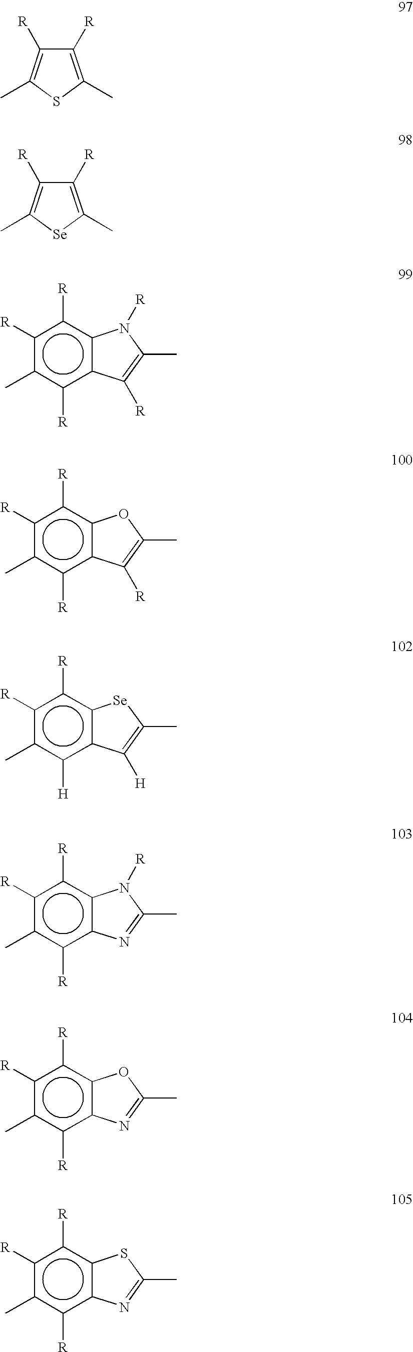

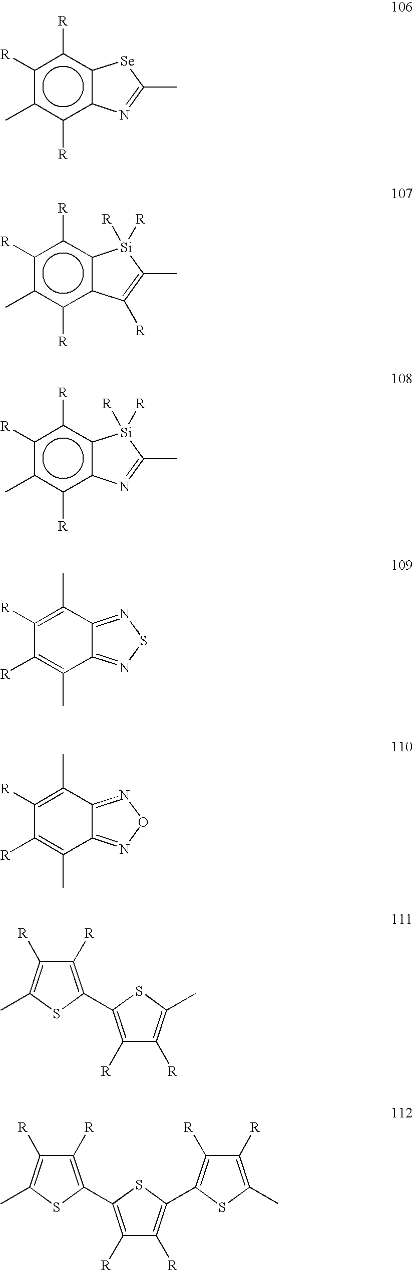

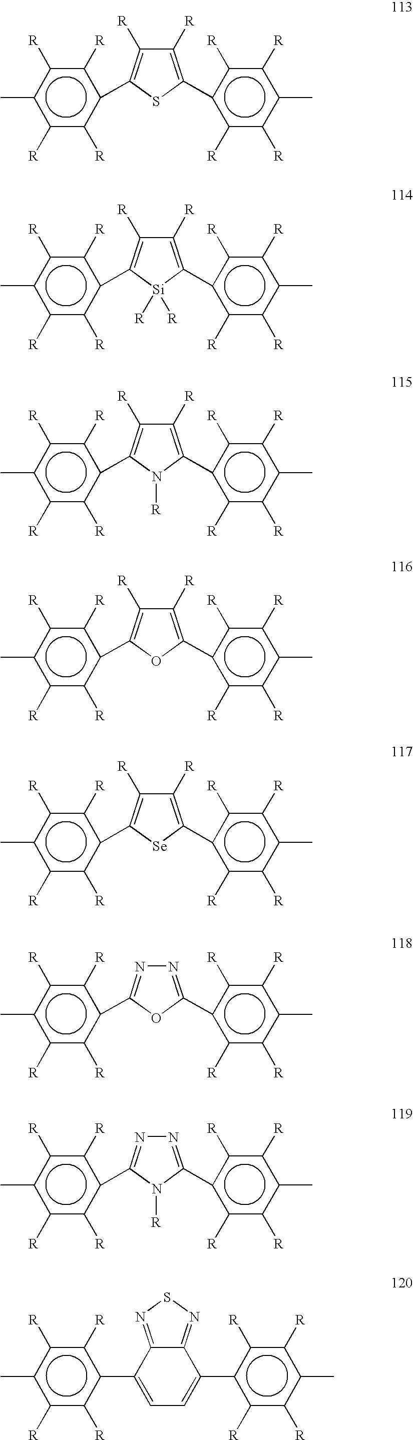

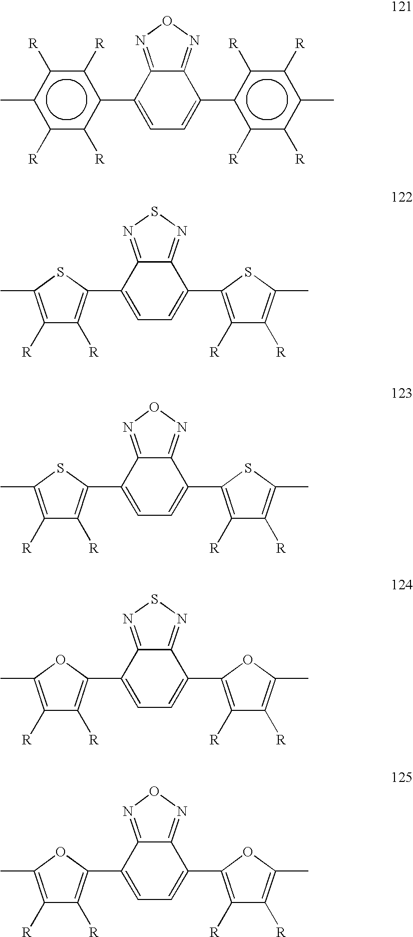

The divalent group having a metal complex structure for Ar₁, Ar₂, Ar₃ and Ar₄ is a divalent group remaining after removing two hydrogen atoms from an organic ligand of a metal complex having an organic ligand.

The organic ligand has a carbon number of usually about from 4 to 60, and examples thereof include 8-quinolinol and derivatives thereof, benzoquinolinol and derivatives thereof, 2-phenyl-pyridine and derivatives thereof, 2-phenyl-benzothiazole and derivatives thereof, 2-phenyl-benzoxazole and derivatives thereof, porphyrin and derivatives thereof, and the like.

The central metal of the complex includes, for example, aluminum, zinc, beryllium, iridium, platinum, gold, europium, terbium and the like.

The metal complex having an organic ligand includes metal complexes, triplet light emitting complexes and the like known as fluorescent materials and phosphorescence materials of lower molecular weight.

The example of divalent group having a metal complex structure includes the following (126 to 132).

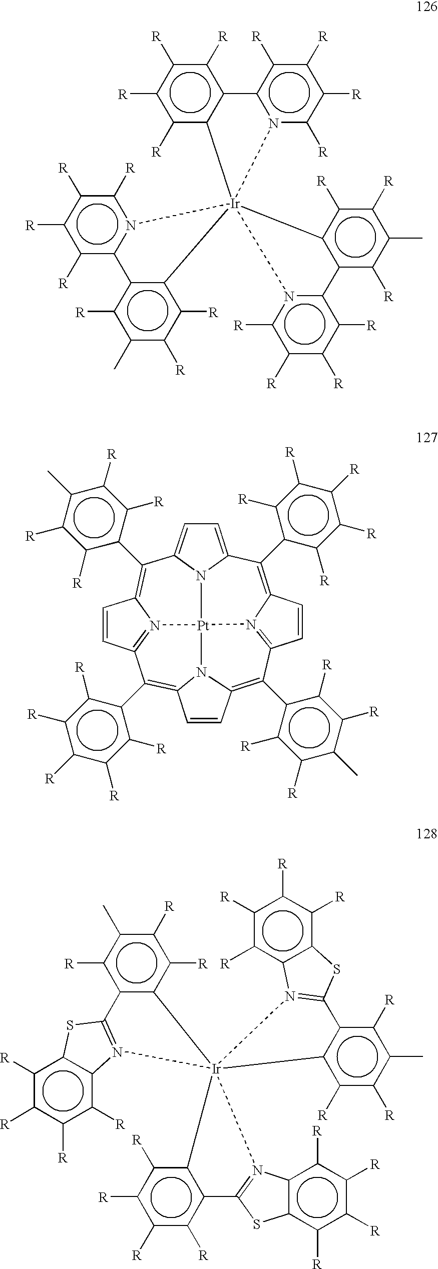

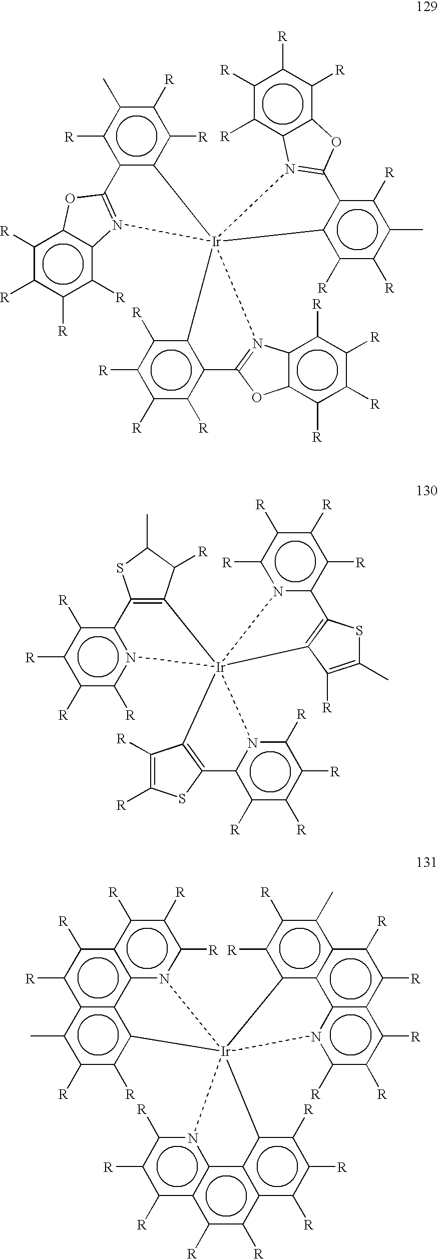

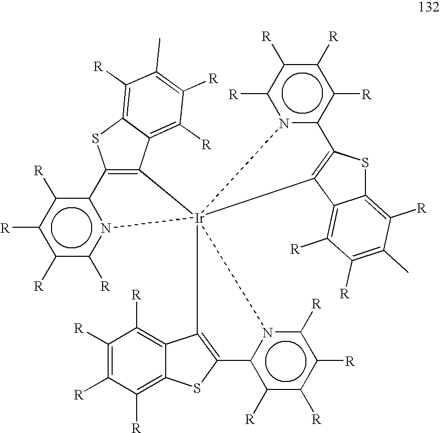

In the above-described formulae 1 to 132, Rs represent each independently a hydrogen atom, alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group, nitro group or cyano group. A carbon atom in groups of the formulae 1 to 132 may be substituted by a nitrogen atom, oxygen atom or sulfur atom, and a hydrogen atom in the groups may be substituted by a fluorine atom.

Here, the definitions, examples and preferable examples of the alkyl group, alkoxy group, alkylthio group, aryl group, aryloxy group, arylthio group, arylalkyl group, arylalkoxy group, arylalkylthio group, arylalkenyl group, arylalkynyl group, substituted amino group, substituted silyl group, halogen atom, acyl group, acyloxy group, imine residue, amide group, acid imide group, monovalent heterocyclic group and substituted carboxyl group are the same as those when the above-described aromatic hydrocarbon ring has a substituent.

From the standpoint of solubility, device properties and the like, the above-described formula (8) is preferably a repeating unit of the following formula (12).

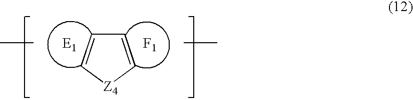

(wherein, it is preferable that a ring E1 and a ring F1 represent each independently a benzene ring or naphthalene ring, and it is further preferable that all of the ring E1 and the ring F1 represent a benzene ring. Two bonds are present on the ring E1 or the ring F1. Z₄ represents —C(Rₐ)(R_{b})—, >C═C(Rₐ)(R_{b}), —O—, —S—, —S(═O)—, —S(═O)(═O)—, —N(Rₐ)—, —Si(Rₐ)(R_{b})—, —P(═O)(Rₐ)—, —P(Rₐ)—, —B(Rₐ)—, —C(Rₐ)(R_{b})—O—, —C(═O)—O—, —C(Rₐ)═N— or —Se—. (Rₐ) and (R_{b}) represent each independently a substituent.).

Examples of structures in which Z4 is carbon in a repeating unit of the above-described formula (12) include the following structures (12-1 to 12-73) and these structures having a substituent. Examples of the kind of the substituent on the ring E1 and the ring F1, include the same groups as the above-described substituents on the rings A to C.

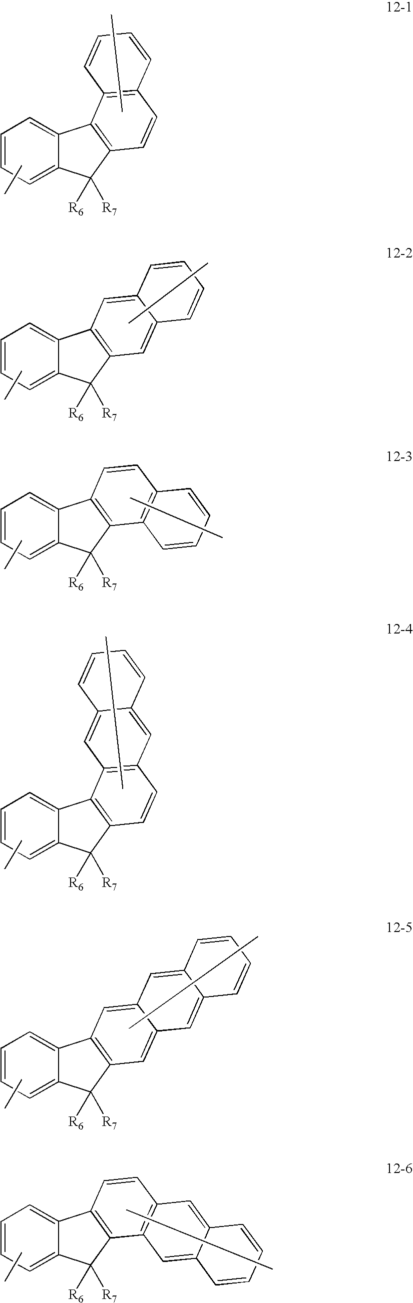

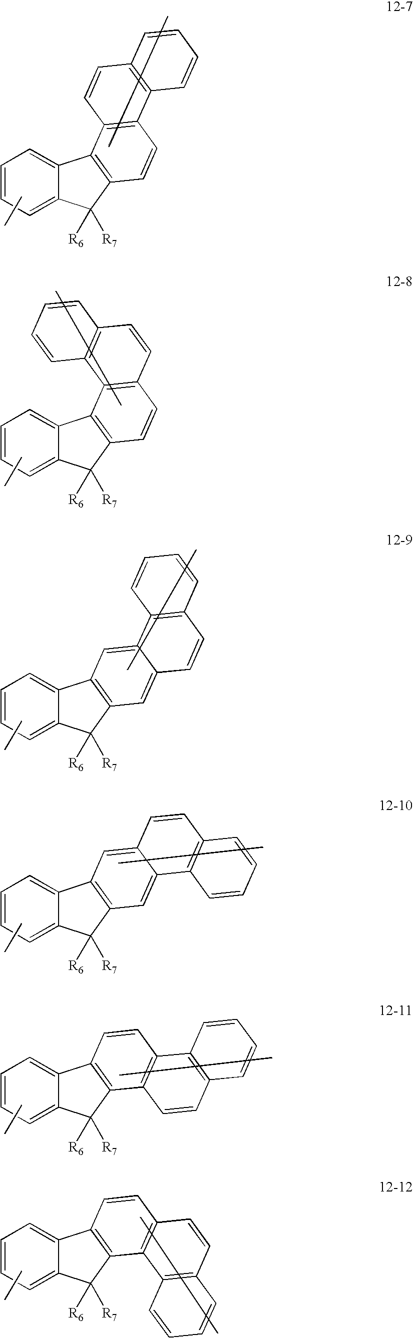

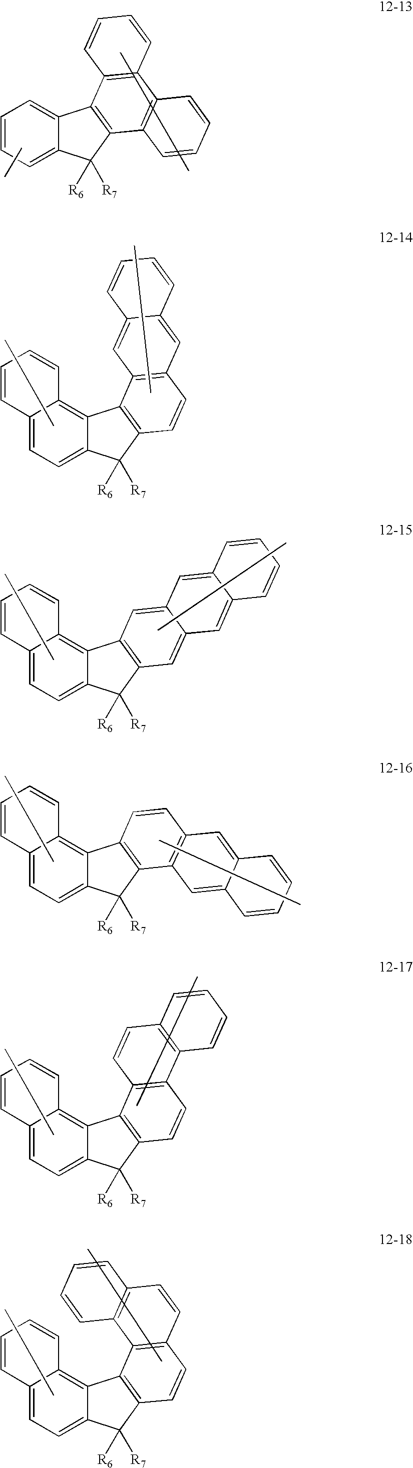

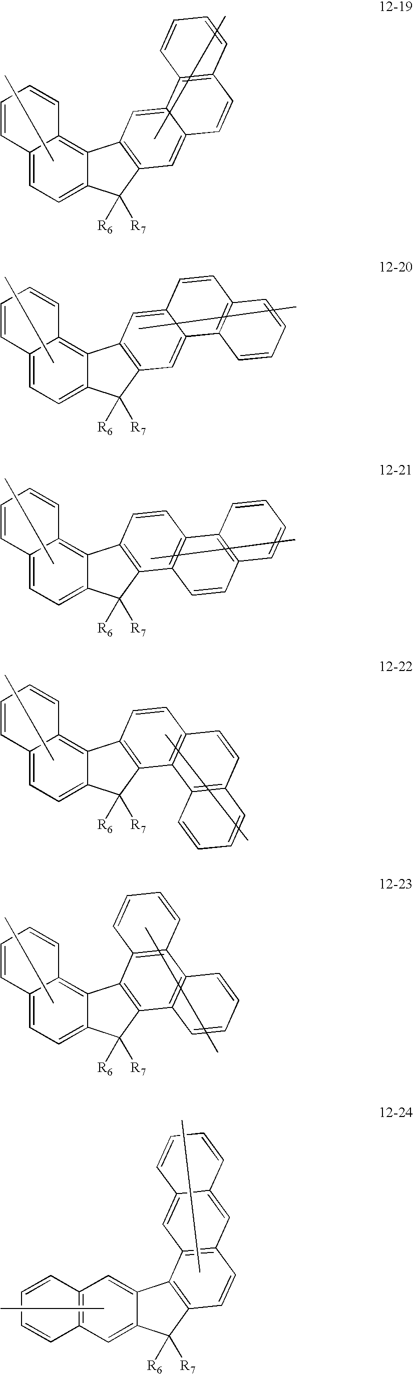

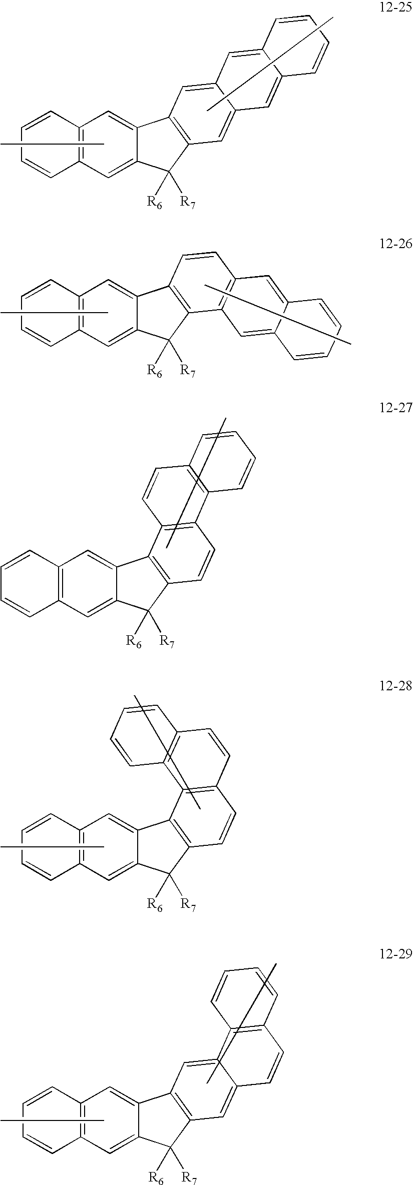

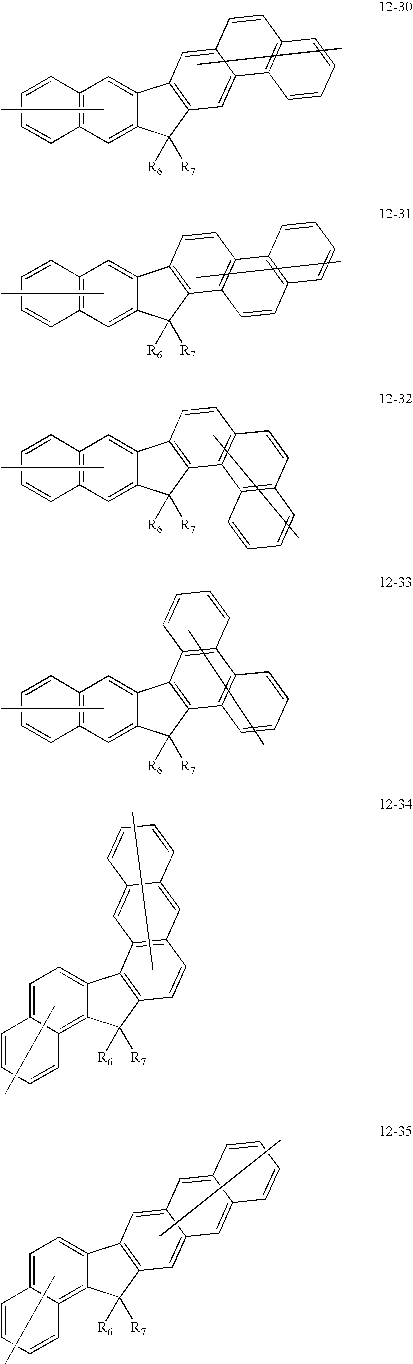

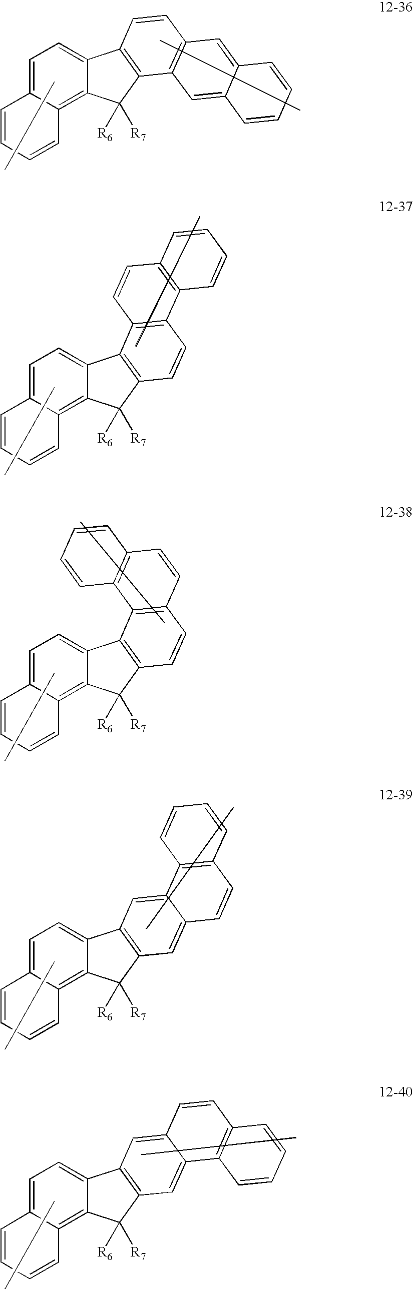

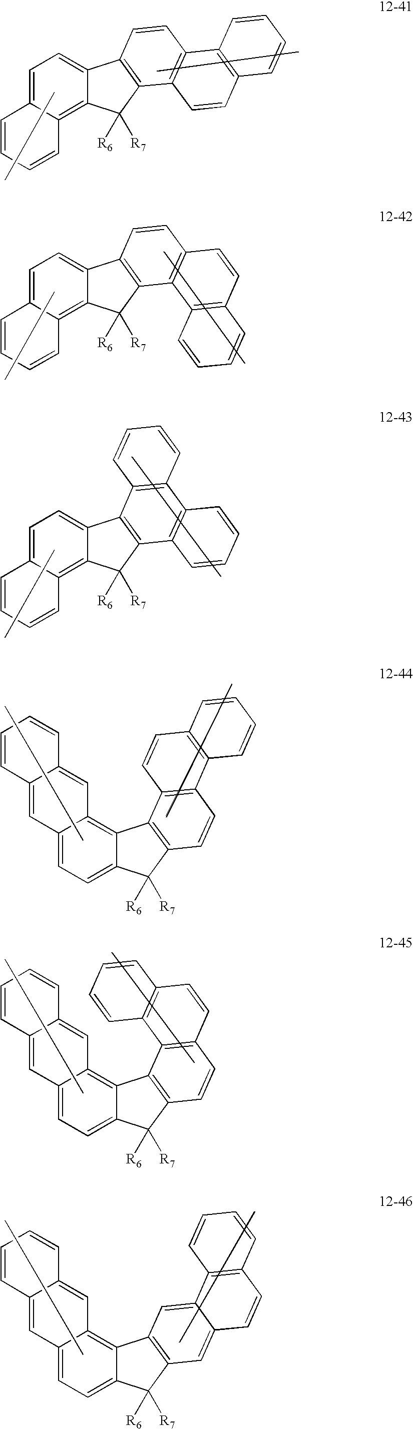

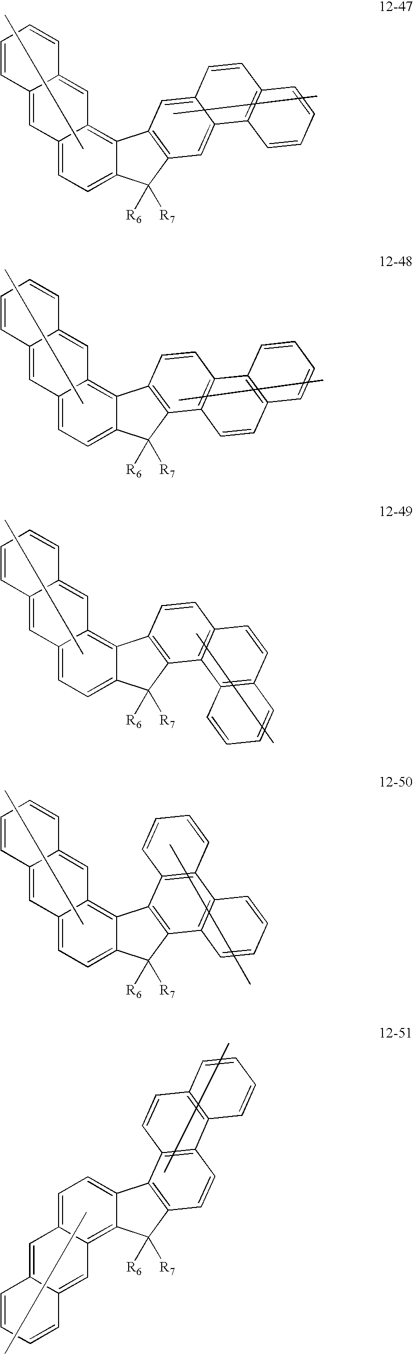

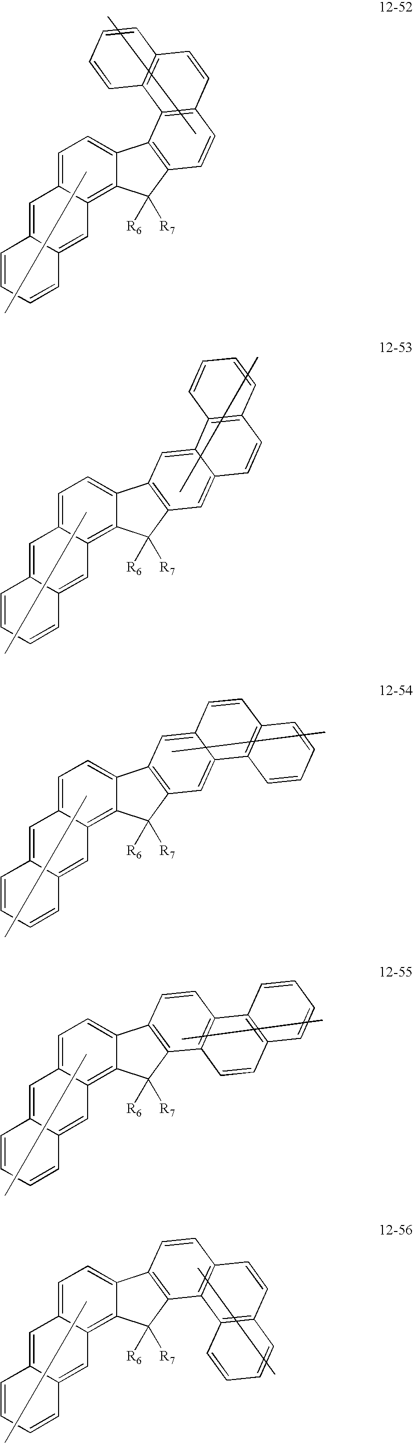

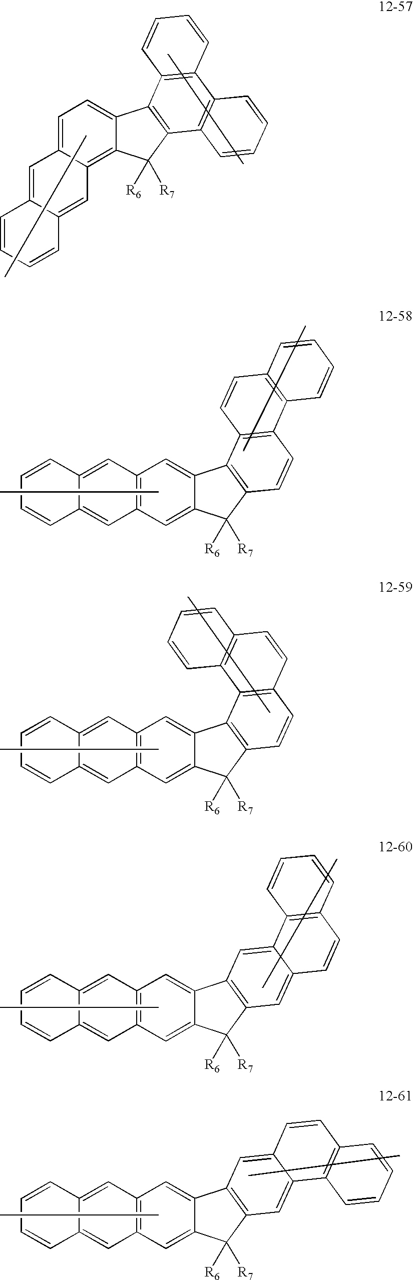

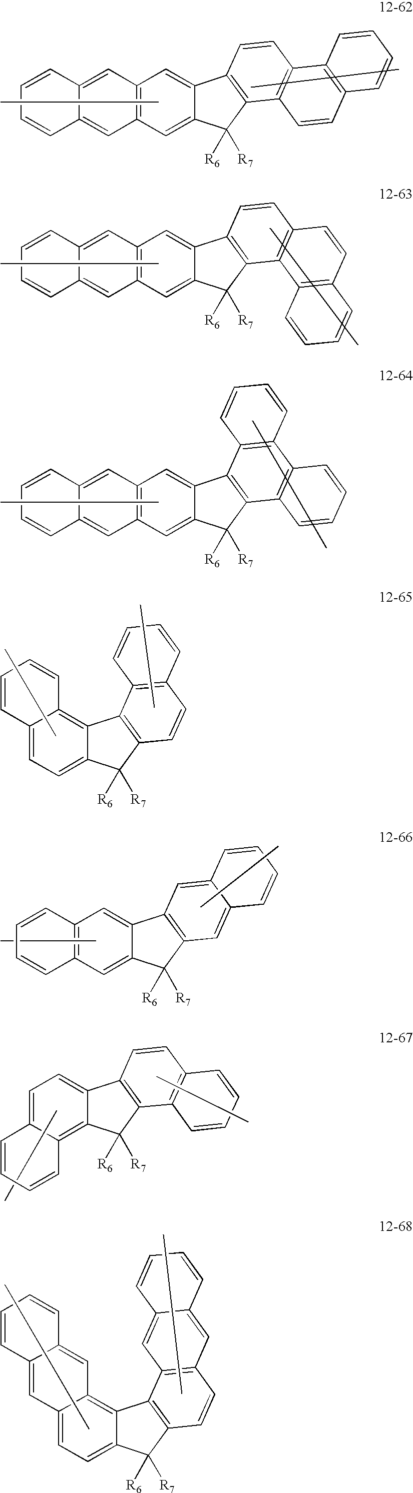

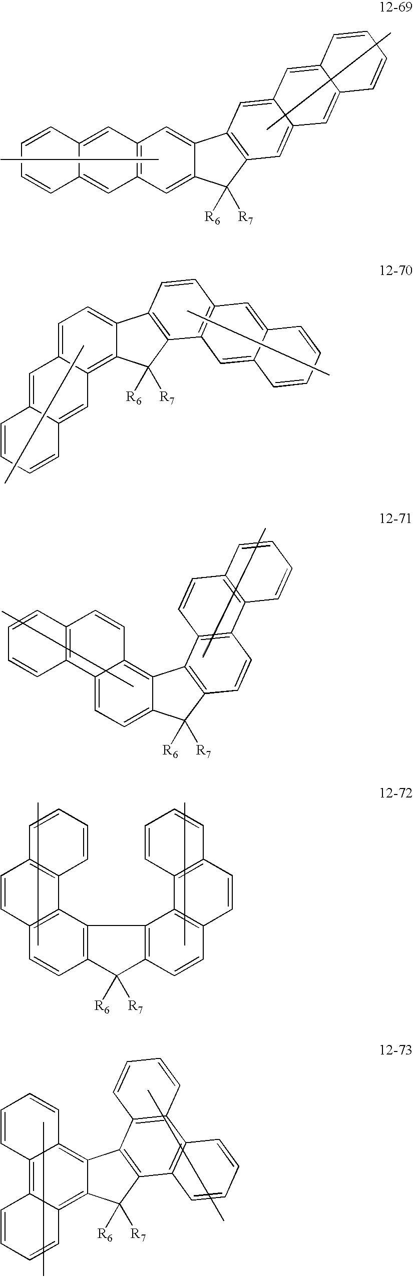

In the above-described formulae, Ra, Rb, and R₆ to R₇ represent each independently a substituent. The substituent includes an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, nitro group, amide group, acid imide group, monovalent heterocyclic group, carboxyl group, substituted carboxyl group or cyano group, and the like.

Examples of structures in which Z4 is an atom other than carbon in a repeating unit of the above-described formula (12) are the following structures (12-74 to 12-85) and these structures having a substituent. Examples of the kind of the substituent include the same groups as the above-described substituents on the ring E and the ring F.

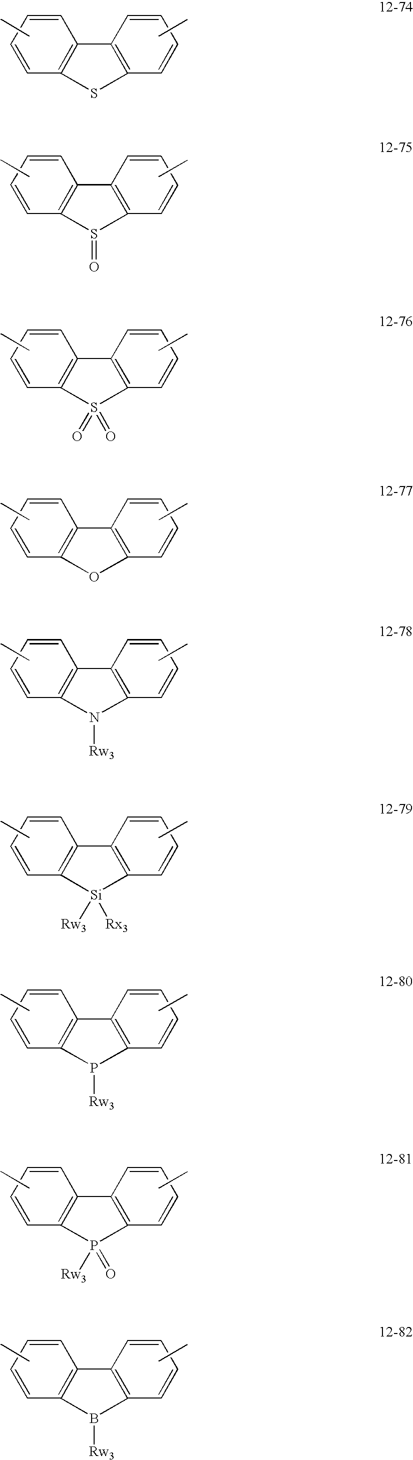

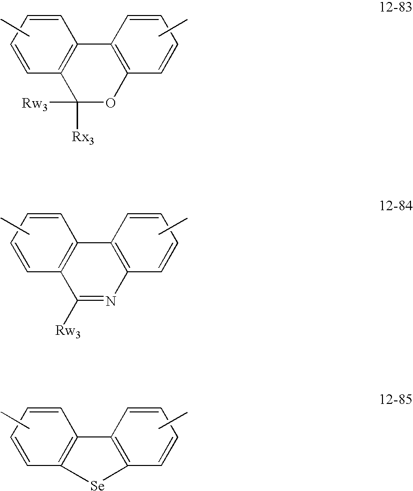

(In the above-described formulae, Rw3 and Rx3 represent each independently a substituent. The substituent includes an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, nitro group, amide group, acid imide group, monovalent heterocyclic group, carboxyl group, substituted carboxyl group or cyano group, and the like.)

The repeating unit of the above-described formula (8) preferably includes repeating units of the following formulae (13) to (19).

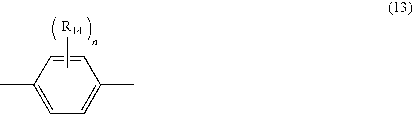

(wherein, R₁₄ represents an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group or cyano group. n represents an integer of 0 to 4. When there are two or more R₁₄s, they may be the same or different.)

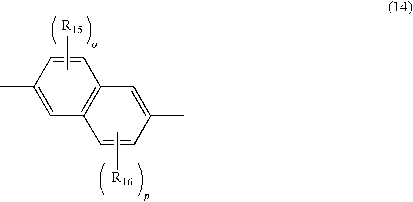

(wherein, R₁₅ and R₁₆ represent each independently an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group or cyano group. o and p represent each independently an integer of 0 to 3. When there are two or more R₁₅s or R₁₆s, respectively, they may be the same or different.)

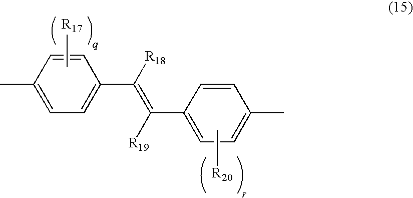

(wherein, R₁₇ and R₂₀ represent each independently an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group or cyano group. q and r represent each independently an integer of 0 to 4. R₁₈ and R₁₉ represent each independently a hydrogen atom, alkyl group, aryl group, monovalent heterocyclic group, carboxyl group, substituted carboxyl group or cyano group. When there are two or more R₁₇s or R₂₀s, they may be the same or different.)

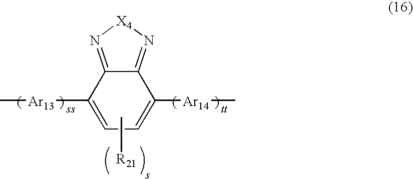

(wherein, R₂₁ represents an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group or cyano group. s represents an integer of 0 to 2. Ar₁₃ and Ar₁₄ represent each independently an arylene group, divalent heterocyclic group or divalent group having a metal complex structure. ss and tt represent each independently 0 or 1.

X₄ represents O, S, SO, SO₂, Se or Te. When there are two or more R₂₁s, they may be the same or different.)

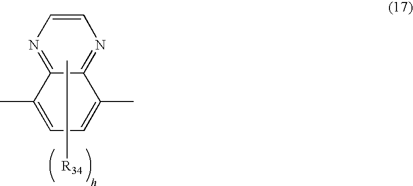

(wherein, R₃₄ represents an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group or cyano group. h represents an integer of 0 to 4. When there are two or more R₃₄s, they may be the same or different.)

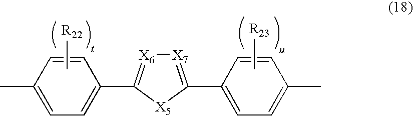

(wherein, R₂₂ and R₂₅ represent each independently an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group or cyano group. t and u represent each independently an integer of 0 to 4. X₅ represents O, S, SO₂, Se, Te, N—R₂₄ or SiR₂₅R₂₆. X₆ and X₇ represent each independently N or C—R₂₇. R₂₄, R₂₅, R₂₆ and R₂₇ represent each independently a hydrogen atom, alkyl group, aryl group, arylalkyl group or monovalent heterocyclic group. When there are two or more R₂₂, R₂₃ or R₂₇, they may be the same or different.).

Examples of the center 5-membered ring in a repeating unit of the formula (18) include thiadiazole, oxadiazole, triazole, thiophene, furan, silole and the like.

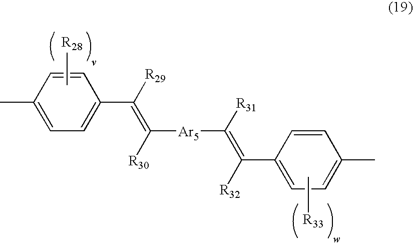

(wherein, R₂₈ and R₃₃ represent each independently an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group or cyano group. v and w represent each independently an integer of 0 to 4. R₂₉, R₃₀, R₃₁ and R₃₆ represent each independently a hydrogen atom, alkyl group, aryl group, monovalent heterocyclic group, carboxyl group, substituted carboxyl group or cyano group. Ar₅ represents an arylene group, divalent heterocyclic group or divalent group having a metal complex structure. When there are two or more R₂₈ or R₃₃, they may be the same or different.).

Among repeating units of the above-described formula (9), repeating units of the following formula (20) are preferable from the standpoints of variation of light emission wavelength, enhancement of light emission efficiency, and improvement in heat resistance.

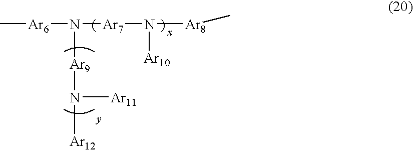

(wherein, Ar₆, Ar₇, Ar₈ and Ar₉ represent each independently an arylene group or divalent heterocyclic group. Ar₁₀, Ar₁₁ and Ar₁₂ represent each independently an aryl group or monovalent heterocyclic group. Ar₆, Ar₇, Ar₈, Ar₉, Ar₁₀, Ar₁₁ and Ar₁₂ may have a substituent. x and y represent each independently 0 or a positive integer.).

From the standpoints of stability of a light emitting device and easiness of synthesis, it is preferable that 1 or more and 3 or less repeating units of the above-described formula (20) are contained, and it is more preferable that 1 or 2 repeating units are contained. Further preferable is a case of containing one repeating unit of the formula (20).

Among conjugated polymer compounds of the present invention, preferable is a case containing two repeating units of the above-described formula (20) as a repeating units, and preferable from the standpoints of regulation of light emission wavelength, device properties and the like is a case containing a combination of a repeating unit in which x=y=0 and a repeating unit in which x=1 and y=0, or a combination of two repeating units in which x=1 and y=0.

In the present invention, when a repeating unit of the above-described formula (2) or (3) and a repeating unit of the above-described formula (9) are contained, the molar ratio thereof is preferably 98:2 to 60:40.

From the standpoints of fluorescence intensity, device properties and the like, it is more preferable that the proportion of a repeating unit of the above-described formula (9) based on the sum of a repeating unit of the above-described formula (2) or (3) and a repeating unit of the above-described formula (20) is 30 mol % or less. In the case of producing a device for EL using only one conjugated polymer compound of the present invention, the ratio of a repeating unit of the above-described formula (2) or (3) to a repeating unit of the above-described formula (20) is preferably 95:5 to 70:30, from the standpoints of device properties and the like.

In the present invention, when a repeating unit of the above-described formula (2) or (3) and a repeating unit of the above-described formula (8), (10) or (11) are contained, the molar ratio thereof is preferably 90:10 to 10:90.

In the present invention, when a repeating unit of the above-described formula (2) or (3) and repeating units of the above-described formulae (8) to (11) (excluding a case in which the above-described formula (8) is the above-described formula (8), (9) or (11), and a case in which the above-described formula (9) is the above-described formula (20)) are contained, the molar ratio thereof is preferably 99:1 to 60:40, more preferably 99:1 to 70:30.

Examples of a repeating unit of the above-described formula (9) include those of the following formulae (133 to 140).

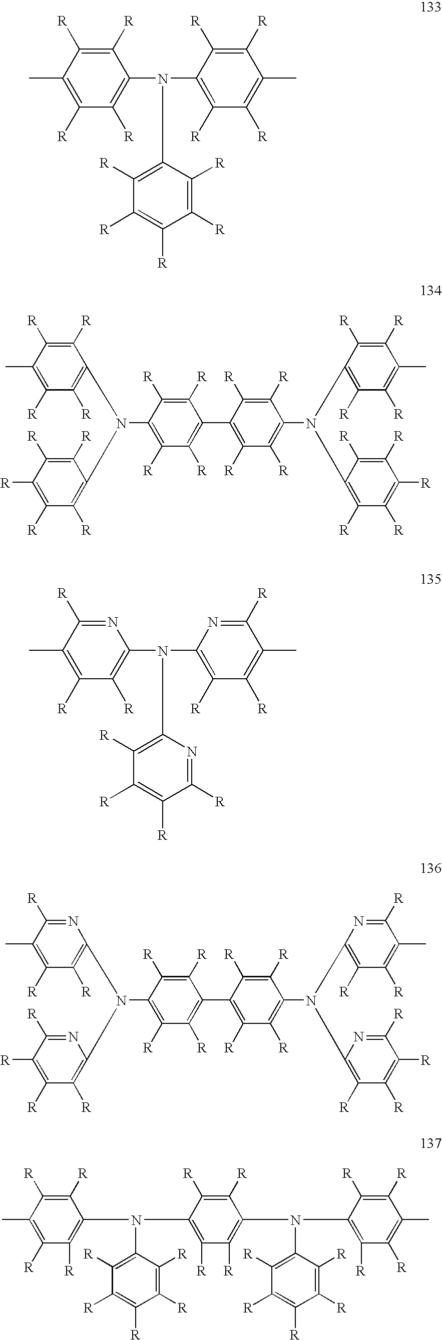

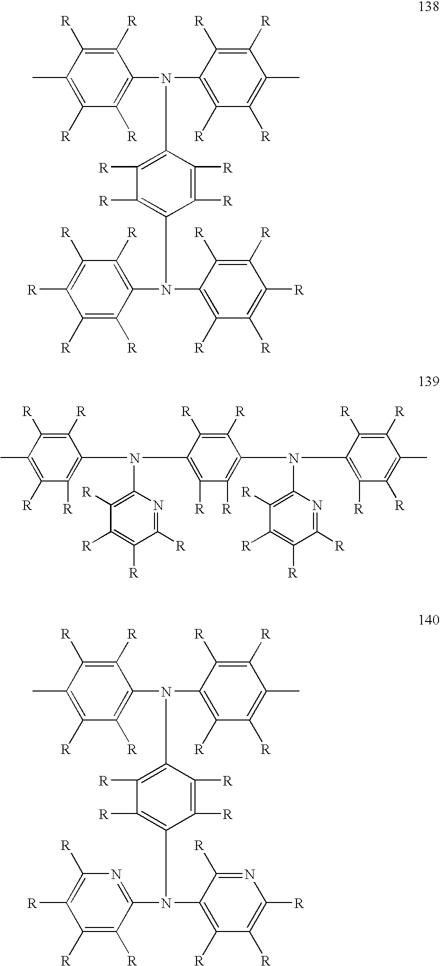

In the above-described formulae, R has the same meaning as in the above-described formulae 1 to 132. For enhancing solubility in organic solvents, it is preferable that at least one group other than a hydrogen atom is contained, and it is preferable that the form of a repeating unit including a substituent shows small symmetry.

When R is a substituent containing alkyl in the above-described formula, it is preferable that cyclic or branched alkyl is contained in at least one substituent for enhancing solubility of a conjugated polymer compound in organic solvents. Further, when R contains partially an aryl group or heterocyclic group in the above-described formula, these may further have at least one substituent. Among structures of the above-described formulae 133 to 140, structures of the above-described formula 134 and the above-described formula 137 are preferable from the standpoint of regulation of light emission wavelength.

In the repeating unit of the above-described formula (20), it is preferable that Ar₆, Ar₇, Ar₈ and Ar₉ represent each independently an arylene group and Ar₁₀, Ar₁₁ and Ar₁₂ represent each independently an aryl group, from the standpoints of regulation of light emission wavelength, device properties and the like.

It is preferable that Ar₆, Ar₇ and Ar₈ represent each independently an un-substituted phenylene group, un-substituted biphenyl group, un-substituted naphthylene group, or un-substituted anthracene-diyl group.

From the standpoints of solubility in organic solvents, device properties and the like, it is preferable that Ar₁₀, Ar₁₁ and Ar₁₂ represent each independently an aryl group having 3 or more substituents, it is more preferable that Ar₁₀, Ar₁₁ and Ar₁₂ represent a phenyl group having 3 or more substituents, naphthyl group having 3 or more substituents or anthranyl group having 3 or more substituents, it is further preferable that Ar₁₀, Ar₁₁ and Ar₁₂ represent a phenyl group having 3 or more substituents.

Particularly, it is preferable that Ar₁₀, Ar₁₁ and Ar₁₂ represent each independently group of the following formula (35) and x+y=3, it is more preferable that x+y=1, it is further preferable that x=1 and y=0.

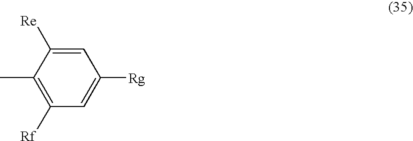

(wherein, Re, Rf and Rg represent each independently an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, silyloxy group, substituted silyloxy group, monovalent heterocyclic group or halogen atom. A hydrogen atom contained in Re, Rf and Rg may be substituted by a fluorine atom.).

More preferably, in the above-described formula (35), Re and Rf represent each independently an alkyl group having 3 or less carbon atoms, alkoxy group having 3 or less carbon atoms or alkylthio group having 3 or less carbon atoms and Rg represents an alkyl group having 3 to 20 carbon atoms, alkoxy group having 3 to 20 carbon atoms or alkylthio group having 3 to 20 carbon atoms.

In the repeating unit of the above-described formula (20), Ar₇ is preferably the following formula (36-1) or (36-2).

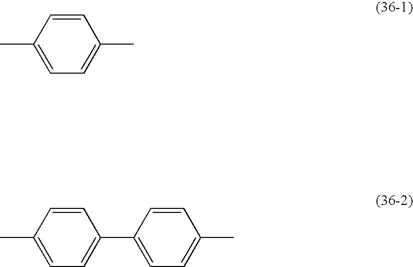

(wherein, benzene rings contained in the structures of (36-1) and (36-2) may have each independently 1 to 4 substituents. These substituents may be mutually the same or different. Two or more of substituents may be connected to form a ring. Further, another aromatic hydrocarbon ring or heterocyclic ring may be bonded next to the benzene ring.).

Particularly preferable examples of the repeating unit of the above-described formula (20) include those of the following formulae (141 to 142).

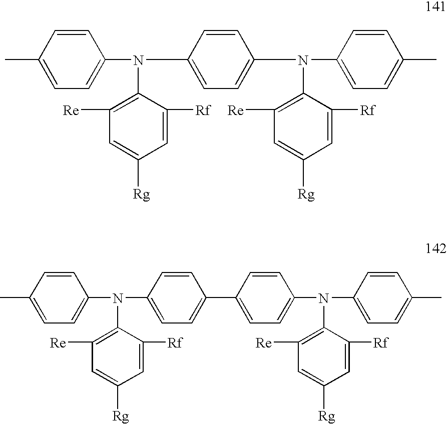

As the conjugated polymer compound containing a repeating unit other than the repeating unit of the above-described formula (2) or (3), preferable are those containing at least one repeating unit selected from repeating units of the above-described formulae (6) and (7) and at least one repeating unit of the above-described formulae (12), (14) to (20), more preferable are those containing any one of repeating units of the formulae 133, 134, 137 and 138 and a repeating unit of the formula (6) or (7), and further preferable are those containing any one of repeating units of the formulae 134 and 137 and a repeating unit of the formula (6) or (7), from the standpoints of fluorescence property, device properties and the like.

Preferable among conjugated polymer compounds of the present invention are those in which all bonds between aromatic rings constituting the main chain are substantially direct bonds, or formed via —O—, —N(R)— (R represents a substituent), —S—, —CR═CR— or —C≡C—.

As the conjugated polymer compound of the present invention, preferable are those containing at least one repeating unit selected from repeating units of the above-described formulae (6) and (7) and at least one repeating unit of the above-described formulae (12), (14) to (20), more preferable are those containing any one of repeating units of the formulae 133, 134, 137 and 138 and a repeating unit of the formula (6) or (7), and further preferable are those containing any one of repeating units of the formulae 134 and 137 and a repeating unit of the formula (6) or (7), from the standpoints of a fluorescence property, device properties and the like.

Next, the method for producing a conjugated polymer compound of the present invention will be illustrated.

Among conjugated polymer compounds of the present invention, conjugated polymer compounds having a repeating unit of the formulae (2) to (3) can be produced, for example, by polymerizing a compound of the formula (27) as one of raw materials.

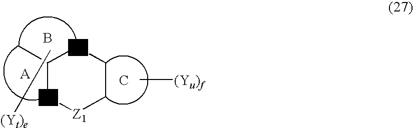

(wherein, a ring A, a ring B, a ring C and Z₁ to Z₃ are as described above. Yₜ and Yᵤ represent each independently a substituent participating in condensation polymerization. and f represent an integer of 0 or more, and e+f=1 and e=2, f=1.). From the standpoints of easiness of increase in polymerization degree and easiness of control of polymerization, it is preferable to perform polymerization using a compound of the following formulae (38) and (39), among compounds of the above-described formula (27).

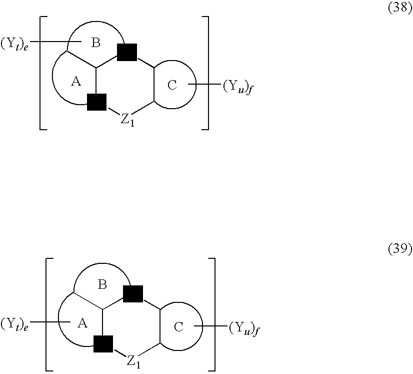

(wherein, a ring A, a ring B and a ring C are as described above. Yₜ and Yᵤ represent each independently a substituent, and Yₜ is connected to the ring A or ring B and Yᵤ is connected to the ring C.).

Raw materials of the conjugated polymer compound having a repeating unit of the formulae (6) and (7) include compounds of the following formulae (28) and (29). A compound of the formula:

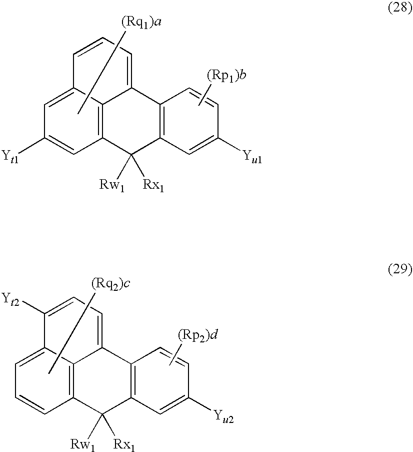

(wherein, R_{w1}, Rₓ₁, R_{w2} and Rₓ₂ represent each independently an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group, nitro group or cyano group, a and c represent an integer of 0 to 5, b and d represent an integer of 0 to 3, and when there are two or more Rₚ₁s, R_{q1}s, Rₚ₂s or R_{q2}s, respectively, they may be the same or different. Yᵤ, Yᵤ₁, Yₜ₂ and Yᵤ₂ represent each independently a substituent participating in condensation polymerization.) can be polymerized as one of raw materials to attain production.

A conjugated polymer compound containing a repeating unit of the above-described formula (29) is preferable from the standpoint of simplicity of synthesis.

The definitions and examples of the alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group and substituted carboxyl group for R_{w1}, Rₓ₁, R_{w2} and Rₓ₂ and Rₚ₁, R_{q1}, Rₚ₂ and R_{q2} are the same as definitions and examples thereof for substituents when a ring A and a ring B in the above-described formula (1) have a substituent.

It is preferable that substituents participating in polymerization for Yₜ, Yᵤ, Yₜ₁, Yᵤ₁, Yₜ₂ and Yᵤ₂ are selected each independently from halogen atoms, alkyl sulfonate groups, aryl sulfonate groups and aryl alkyl sulfonate groups, since the polymer compound is easy to synthesis and can be used as a raw material for various polymerization reactions.

It is preferable that Yₜ₁ and Yᵤ₁ represent a bromine atom in (28), since the polymer compound is easy to synthesis, manifests easy functional group conversion and can be used as a raw material for various polymerization reactions.

It is preferable that Yₜ₂ and Yᵤ₂ represent a bromine atom in (29), since the polymer compound is easy to synthesis, manifests easy functional group conversion and can be used as a raw material for various polymerization reactions.

From the standpoint of improvement in heat resistance in (28), it is preferable that a and b are 0.

From the standpoint of improvement in heat resistance in (29), it is preferable that c and d are 0.

In the case of production of conjugated polymer compounds and dendrimers having branching in the main chain and having three or more end parts, they can be produced by polymerizing a compound of the following formula (40) as one of raw materials. A compound of the formula:

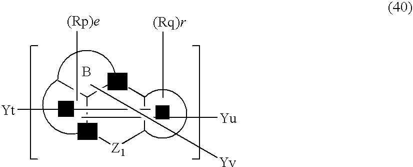

(wherein, Yₜ, Yᵤ and Yᵥ represent a substituent participating in condensation polymerization. e and f represent 0 or a positive integer.) can be polymerized as one of raw materials to attain production.

The raw material of the formula (40) preferably includes compounds of the following formulae (41) and (42).

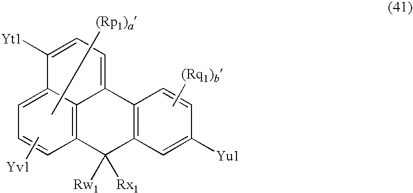

(wherein, R_{w1}, Rₓ₁, Rₚ₁, Yₜ₁, Yᵤ₁ and Yᵥ₁ represent each independently a substituent, a′ represents an integer of 0 to 4, and b′ represents an integer of 0 to 3. When there are two or more Rₚ₁s or R_{q1}s, respectively, they may be the same or different.).

The raw material of the formula (40) preferably includes compounds of the following formula (42).

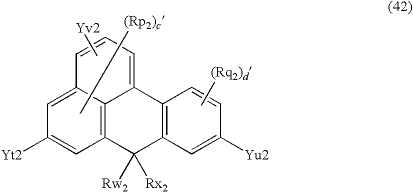

(wherein, R_{w2}, Rₓ₂, Rₚ₂, R_{q2}, Yₜ₂, Yᵤ₂ and Yᵥ₂ represent each independently a substituent, c′ represents an integer of 0 to 4, and d′ represents an integer of 0 to 3. When there are two or more Rₚ₂s or R_{q2}s, respectively, they may be the same or different.).

From the standpoint of improvement in heat resistance in (41), it is preferable that a′ and b′ are 0.

From the standpoint of improvement in heat resistance in (42), it is preferable that c′ and d′ are 0.

In the production of a conjugated polymer compound of the present invention, a conjugated polymer compound having higher molecular weight is obtained when a compound of the above-described formula (40) or (41), (42) is contained in raw material monomers. In this case, a compound of the above-described formula (40) or (41), (42) is contained in an amount of preferably 10 mol % or less, further preferably 1 mol % or less in raw material monomers when the amount of a compound of the above-described formula (28) is 100 mol %.

When the conjugated polymer compound of the present invention has a repeating unit other than the formula (2) or (3), it may be advantageous to perform polymerization in the co-existence of a compound having two substituents participating in polymerization as a repeating unit other than the formula (41) or (42).

The compound having two polymerizable substituents as a repeating unit other than the repeating unit of the above-described formula (2) or (3) includes compounds of the following formulae (31) to (34).

By polymerizing a compound of any of the following formulae (31) to (34) in addition to a compound of the above-described formula (40), a conjugated polymer compound can be produced having at least one unit of (8) to (11) respectively in addition to a unit of the above-described formula (2) or (3).

Y₇—Ar₁—Y₈ (31)

Y₉—(Ar₂—X₁)_{f}—Ar₃—Y₁₀ (32)

Y₁₁—Ar₄—X₂—Y₁₂ (33)

Y₁₃—X₃—Y₁₃ (34)

(wherein, Ar₁, Ar₂, Ar₃, Ar₄, ff, X₁, X₂ and X₃ are the same as described above. Y₅, Y₆, Y₇, Y₈, Y₉, Y₁₀, Y₁₁ and Y₁₂ represent each independently a substituent participating in polymerization.).

An end-capped conjugated polymer compound can be produced by polymerizing a compound of the following formulae (43) and (44) as a raw material, in addition to the above-described formulae (38), (39), (28), (29), (40), (41) and (42), and the above-described formulae (31) to (34).

E₁-Y₁₅ (43)

E₂-Y₁₆ (44)

(E₁ and E₂ represent a monovalent heterocyclic ring, an aryl group having a substituent, a monovalent aromatic amine group, or a monovalent group derived from a heterocyclic ring coordinated metal complex, and Y₁₅ and Y₁₆ represent each independently a substituent participating in polymerization.).

The compound having two substituents participating in condensation corresponding to the above-described formula (20) as a repeating unit other than the repeating unit of the above-described formula (2) or (3) includes compounds of the following formula (45).

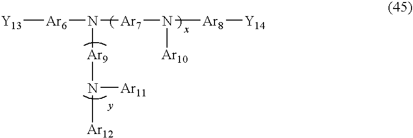

(wherein, the definitions and preferable examples of Ar₆, Ar₇, Ar₈, Ar₉, Ar₁₀, Ar₁₁, Ar₁₂, x and y are the same as described above. Y₁₃ and Y₁₄ represent each independently a substituent participating in polymerization.).

A conjugated polymer having a chain of the following formula (46) can be obtained by copolymerizing a compound of the above-described formula (38) or (39) and a compound of the above-described formula (45).

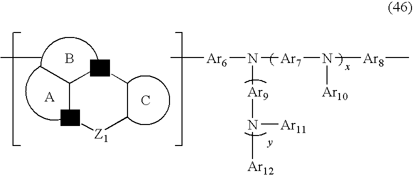

The substituent participating in polymerization in the production method of the present invention includes a halogen atom, alkyl sulfonate group, aryl sulfonate group, aryl alkyl sufonate group; borate group, sulfoniummethyl group, phosphoniummethyl group, phosphonatemethyl group, methyl monohalide group, —B(OH)₂, formyl group, cyano group, vinyl group and the like, among substituents participating in polymerization.

Here, the halogen atom include a fluorine atom, chlorine atom, bromine atom and iodine atom.

Examples of the alkyl sulfonate group include a methane sulfonate group, ethane sulfonate group, trifluoromethane sulfonate group and the like, examples of the aryl sulfonate group include a benzene sulfonate group, p-toluene sulfonate group and the like, and examples of the aryl sulfonate group include benzyl sulfonate group and the like.

The borate group includes groups of the following formulae.

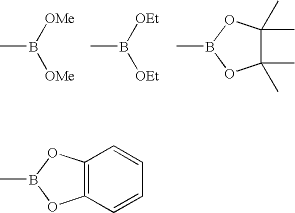

In the formulae, Me represents a methyl group and Et represents an ethyl group.

The sulfoniummethyl group includes groups of the following formulae.

—CH₂S⁺Me₂X⁻, —CH₂S⁺Ph₂X⁻

(wherein, X represents a halogen atom and Ph represents a phenyl group.)

The phosphoniummethyl group includes groups of the following formula.

—CH₂P⁺Ph₃X⁻ (X represents a halogen atom.)

The phosphonatemethyl group includes groups of the following formula.

—CH₂PO(OR′)₂ (X represents a halogen atom, R′ represents an alkyl group, aryl group or arylalkyl group.)

The methyl monohalide group includes a methyl fluoride group, methyl chloride group, methyl bromide group and methyl iodide group.

A preferable substituent as the substituent participating in condensation polymerization varies with the kind of the polymerization reaction, and in the case of use of a 0-valent nickel complex such as, for example, in the Yamamoto coupling reaction and the like, mentioned are halogen atoms, alkyl sulfonate groups, aryl sulfonate group or aryl akyl sulfonate groups. In the case of use of a nickel catalyst or palladium catalyst such as in the Suzuki coupling reaction and the like, mentioned are alkyl sulfonate groups, halogen atoms, borate groups, —B(OH)₂ and the like.

The production method of the present invention can be carried out, specifically, by dissolving a compound having two or more substituents participating in polymerization, as a monomer, in an organic solvent if necessary, and using, for example, an alkali and a suitable catalyst, at temperatures of not lower than the melting point and not higher than the boiling point of the organic solvent. Known methods can be used described, for example, in “Organic Reactions”, vol. 14, p. 270 to 490, John Wiley & Sons, Inc., 1965, “Organic Syntheses”, Collective Volume VI, p. 407 to 411, John Wiley & Sons, Inc., 1988, Chem. Rev., vol. 95, p. 2457 (1995), J. Organomet. Chem., vol. 576, p. 147 (1999), Makromol. Chem., Macromol. Symp., vol. 12, p. 229 (1987), and the like.

In the method for producing a conjugated polymer compound of the present invention, production can be performed by using a known condensation reaction, depending on a substituent participating in condensation polymerization of a raw material compound, as the condensation polymerization method.

When the conjugated polymer compound of the present invention generates a double bond in condensation polymerization, for example, a method described in JP-A No. 5-202355 is mentioned. Namely, polymerization by the Wittig reaction of a compound having a formyl group and a compound having a phosphoniummethyl group, or of a compound having a formyl group and a phosphoniummethyl group, polymerization by the Heck reaction of a compound having a vinyl group and a compound having a halogen atom, polycondensation by a dehydrohalogenation method of a compound having two or more methyl halide groups, polycondensation by a sulfonium salt decomposition method of a compound having two or more methylsulfonium groups, polymerization by the Knoevenagel reaction of a compound having a formyl group and a compound having a cycno group, polymerization by the McMurry reaction of compound having two or more formyl groups, and the like, are illustrated.

When the conjugated polymer compound of the present invention generates a triple bond in the main chain by condensation polymerization, for example, the Heck reaction and the Sonogashira reaction can be utilized.

In the case of no generation of double bond or triple bond, for example, a method of polymerization by the Suzuki coupling reaction from the corresponding monomer, a method of polymerization by the Grignard method, a method of polymerization by a Ni(0) complex, a method of polymerization by an oxidizer such as FeCl₃ and the like, a method of electrochemical oxidation polymerization, a method by decomposition of an intermediate polymer having a suitable leaving group, and the like, are illustrated.

Of them, polymerization by the Wittig reaction, polymerization by the Heck reaction, polymerization by the

Knoevenagel reaction, method of polymerization by the Suzuki coupling reaction, method of polymerization by the Grignard reaction and method of polymerization by a nickel 0-valent complex are preferable since the structure can be controlled easily. Of them, the method of polymerization by a nickel 0-valent complex is preferable from the standpoint of easiness of molecular weight control and from the standpoints of heat resistance and device properties such as life of polymer LED, light emission initiation voltage, current density, increase of voltage in driving, and the like.

Since the conjugated polymer compound of the present invention has an asymmetrical skeleton in its repeating unit as shown in the formula (2) or (3), the orientation of a repeating unit is present in the polymer compound. In the case of control of the orientation of a repeating unit, for example, a method of polymerization in which the orientation of a repeating unit is controlled by selecting a combination of a polymerization reaction to be used and a substituent participating in condensation polymerization of the corresponding monomer, and the like, are illustrated.

In the case of control of a sequence of two or more repeating units in the conjugated polymer compound of the present invention, a method in which an oligomer having part or all of repeating units in the intended sequence is synthesized before polymerization, a method in which substituents participating in condensation polymerization and a polymerization reaction to be used are selected, and a sequence of repeating units is controlled in polymerization, and the like, are illustrated.

In the production method of the present invention, it is preferable that substituents participating in condensation polymerization are selected from halogen atoms, alkyl sulfonate groups, aryl sulfonate groups and aryl alkyl sulfonate groups, and condensation polymerization is carried out in the present of a nickel 0-valent complex.

The raw material compound includes dihalogenated compounds, bis(alkyl sulfonate) compounds, bis(aryl sulfonate) compounds, bis(aryl alkyl sulfonate) compounds or halogen-alkyl sulfonate compounds, halogen-aryl sulfonate compounds, halogen-aryl alkyl sulfonate compounds, alkyl sulfonate-aryl sulfonate compounds, alkyl sulfonate-aryl alkyl sulfonate compounds, and aryl sulfonate-aryl alkyl sulfonate compounds.

In this case, there is mentioned a method in which, a conjugated polymer compound in which the orientation of a repeating unit and a sequence are controlled is produced by using, for example, a halogen-alkyl sulfonate compound, halogen-aryl sulfonate compound, halogen-aryl alkyl sulfonate compound, alkyl sulfonate-aryl sulfonate compound, alkyl sulfonate-aryl alkyl sulfonate compound, and aryl sulfonate-aryl alkyl sulfonate compound as the raw material compound.

Among the production methods of the present invention, preferable is a production method in which substituents participating in condensation polymerization are selected from halogen atoms, alkyl sulfonate groups, aryl sulfonate groups, aryl alkyl sulfonate groups, boric acid group or borate groups, the ratio of the sum (J) of mol numbers of halogen atoms, alkyl sulfonate groups, aryl sulfonate groups and aryl alkyl sulfonate groups to the sum (K) of mol numbers of boric acid group (—B(OH)₂) and borate groups, in all raw material compounds, is substantially 1 (usually, K/J is in a range of 0.7 to 1.2), and condensation polymerization is carried out using a nickel catalyst or palladium catalyst.

Combinations of raw material compounds include combinations of a dihalogenated compound, bis(alkyl sulfonate) compound, bis(aryl sulfonate) compound or bis(aryl alkyl sufonate) compound with a diboric acid compound or diborate compound.

Further mentioned are a halogen-boric acid compound, halogen-borate compound, alkyl sulfonate-boric acid compound, alkyl sulfonate-borate compound, aryl sulfonate-boric acid compound, aryl sulfonate-borate compound, aryl alkyl sulfonate-boric acid compound, aryl alkyl sulfonate-boric acid compound and aryl alkyl sulfonate-borate compound.

In this case, there is mentioned a method in which, a conjugated polymer compound in which the orientation of a repeating unit and a sequence are controlled in produced by using, for example, a halogen-boric acid compound, halogen-borate compound, alkyl sulfonate-boric acid compound, alkyl sulfonate-borate compound, aryl sulfonate-boric acid compound, aryl sulfonate-borate compound, aryl alkyl sulfonate-boric acid compound, aryl alkyl sulfonate-boric acid compound or aryl alkyl sulfonate-borate compound as the raw material compound.

The organic solvent varies with the reaction and compound to be used, and for suppressing a side reaction, in general, it is preferable that a solvent to be used is subjected to a sufficient deoxygenation treatment and the reaction is progressed in an inert atmosphere. Further, it is preferable to perform a dehydration treatment likewise. However, this is not the case when a reaction in a two-phase system with water such as in the Suzuki coupling reaction is carried out.

Examples of the solvent include saturated hydrocarbons such as pentane, hexane, heptane, octane, cyclohexane and the like, unsaturated hydrocarbons such as benzene, toluene, ethylbenzene, xylene and the like, halogenated saturated hydrocarbons such as carbon tetrachloride, chloroform, dichloromethane, chlorobutane, bromobutane, chloropentane, bromopentane, chiorohexane, bromohexane, chiorocyclohexane, bromocyclohexane and the like, halogenated unsaturated hydrocarbons such as chlorobenzene, dichlorobenzene, trichlorobenzene and the like, alcohols such as methanol, ethanol, propanol, isopropanol, butanol, t-butyl alcohol and the like, carboxylic acids such as formic acid, acetic acid, propionic acid and the like, ethers such as dimethyl ether, diethyl ether, methyl-t-butyl ether, tetrahydrofuran, tetrahydropyran, dioxane and the like, amines such as trimethylamine, triethylamine, N,N,N′,N′-tetramethylethylenediamine, pyridine and the like, amides such as N,N-dimethylformamide, N,N-dimethylacetamide, N,N-diethylacetamide, N-methylmorpholine oxide, and the like, and single solvents or mixed solvents thereof may also be used. Of them, ethers are preferable, and tetrahydrofuran and diethyl ether are further preferable.

For reacting, an alkali or suitable catalyst is appropriately added. These may be advantageously selected depending on the reaction to be used. As the alkali or catalyst, those which are sufficiently dissolved in the solvent used in the reaction are preferable. As the method of mixing an alkali or catalyst, there is, for example, a method in which a solution of an alkali or catalyst is added slowly while stirring the reaction liquid under an inert atmosphere such as argon and nitrogen and the like, or reversely, the reaction liquid is slowly added to a solution of an alkali or catalyst.

The conjugated polymer compound of the present invention may be a random, block or graft copolymer, or a polymer having an intermediate structure, for example, a random copolymer having a block property. From the standpoint of obtaining a polymer light emitter showing high quantum yield of fluorescence or phosphorescence, a random copolymer having a block property and a block or graft copolymer are more preferable than a complete random copolymer. Those having branching in the main chain and thus having 3 or more end parts, and dendrimers are also included.

When two structures of the above-described formula (2) are adjacent, structures of the following formulae (21), (22) and (23) are obtained. From the standpoints of electron injection property and transportability, it is preferable that at least one of (21) to (23) is contained in a conjugated polymer compound.

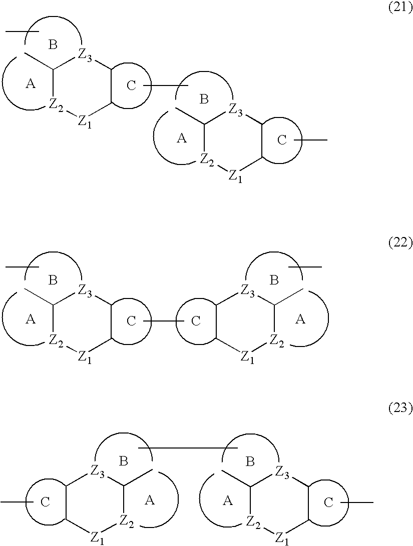

When two structures of the above-described formula (3) are adjacent, structures of the following formulae (24), (25) and (26) are obtained. From the standpoints of electron injection property and transportability, it is preferable that at least one of (25) to (27) is contained in a conjugated polymer compound.

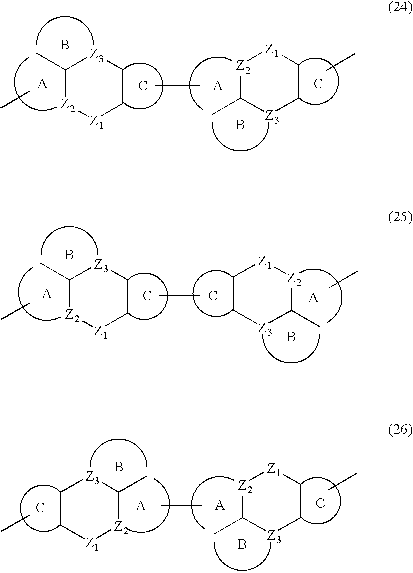

To be capable of standing various processes for producing a light emitting device and the like, a conjugated polymer compound has a glass transition temperature of preferably 100° C. or higher, more preferably 130° C. or higher, further preferably 150° C. or higher.

The conjugated polymer compound of the present invention has a polystyrene-reduced number-average molecular weight of usually about from 10³ to 10⁸, preferably 10⁴ to 10⁶. The polystyrene-reduced weight-average molecular weight is usually about from 10³ to 10⁸, and from the standpoint of film formability and from the standpoint of efficiency in the case of making a device, preferably 5×10⁴ or more, further preferably 10⁵ or more. From the standpoint of solubility, it is preferably 10⁵ to 5×10⁶. In the case of conjugated polymer compounds having a molecular weight in a preferable range, even if the compound is used singly in a device or two or more of them are mixed and used in a device, high efficiency is obtained. Likewise, from the standpoint of enhancing film formability of a conjugated polymer compound, the degree of dispersion (weight-average molecular weight/number-average molecular weight) is preferably 1.5 or more.

The conjugated polymer compound of the present invention may have a branched structure in the main chain, and the branched structure includes cases containing a structure of the above-described formula (5), and a case containing at least one bond in a ring B and at least one bond in a ring C is preferable.

As the branched structure, a case of the following formula (37) is further preferable.

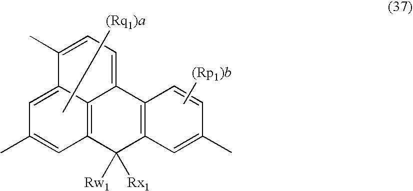

(wherein, Rₚ₁, R_{q1}, R_{w1} and Rₓ₁ represent the same meanings as described above. a represents an integer value of 0 to 4, and b represents an integer value of 0 to 3.).

An end group of the conjugated polymer compound of the present invention is preferably protected by a stable group since when a polymerization active group remains intact, there is a possibility of decrease in light emission property and life when made into a device. A structure containing a conjugation bond continuous with a conjugation structure of the main chain is preferable, and for example, a structure bonding to an aryl group or heterocyclic group via a carbon-carbon bond is illustrated. Specific examples are substituents described in chemical formula 10 in JP-A No. 9-45478, and the like.

In the conjugated polymer compound of the present invention, it is preferable that at least one of its molecule chain ends has an aromatic end group selected from monovalent heterocyclic groups, monovalent aromatic amine groups, monovalent groups derived from heterocyclic ring coordinated metal complexes and aryl groups having a formula weight of 90 or more. The aromatic end groups may be present singly or in combination. The ratio of end groups other than the aromatic end group is preferably 30% or less, more preferably 20% or less, further preferably 10% or less based on all end groups, and substantially no presence is more preferable, from the standpoints of a fluorescent property and device properties. Here, the molecular chain end means an aromatic end group present at the end of a conjugated polymer compound according to the production method of the present invention, a leaving group of a monomer used for polymerization which remains at the end of a polymer compound, or a proton bonded instead of an aromatic end group though a leaving group of a polymer has departed in a monomer present at the end of a conjugated polymer compound. If a conjugated polymer compound of the present invention is produced using a monomer having a leaving group of a monomer used for polymerization which remains at the end of a conjugated polymer compound, among these molecule chain ends, for example, a monomer having a halogen atom, as a raw material, then, there is a tendency of decrease in a fluorescent property and the like if a halogen remains at the end of the conjugated polymer compound, thus, it is preferable that a leaving group of a monomer does not substantially remain at the end.

In the conjugated polymer compound of the present invention, by capping at least one of its molecule chain ends with an aromatic end group selected from monovalent heterocyclic groups, monovalent aromatic amine groups, monovalent groups derived from heterocyclic ring coordinated metal complexes and aryl groups having a formula weight of 90 or more, it is expected to impart various properties to the conjugated polymer compound. Specifically mentioned are an effect of elongating time necessary for decrease in brilliance of a device, an effect of enhancing charge injection property, charge transportability, light emission property and the like, an effect of enhancing compatibility and mutual action between copolymers, an anchor-like effect, and the like.

The monovalent heterocyclic group includes the above-described groups, and specifically, the following structures are illustrated.

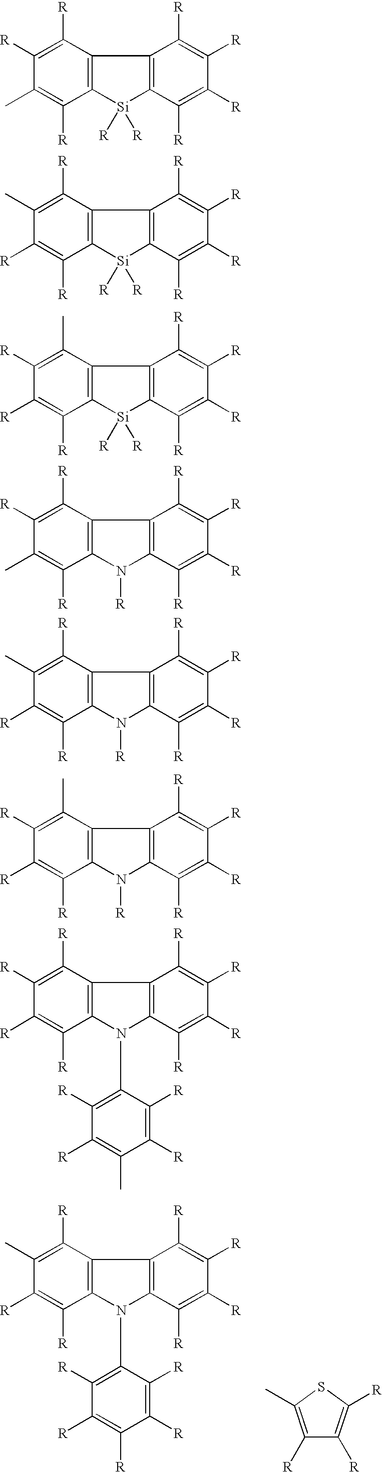

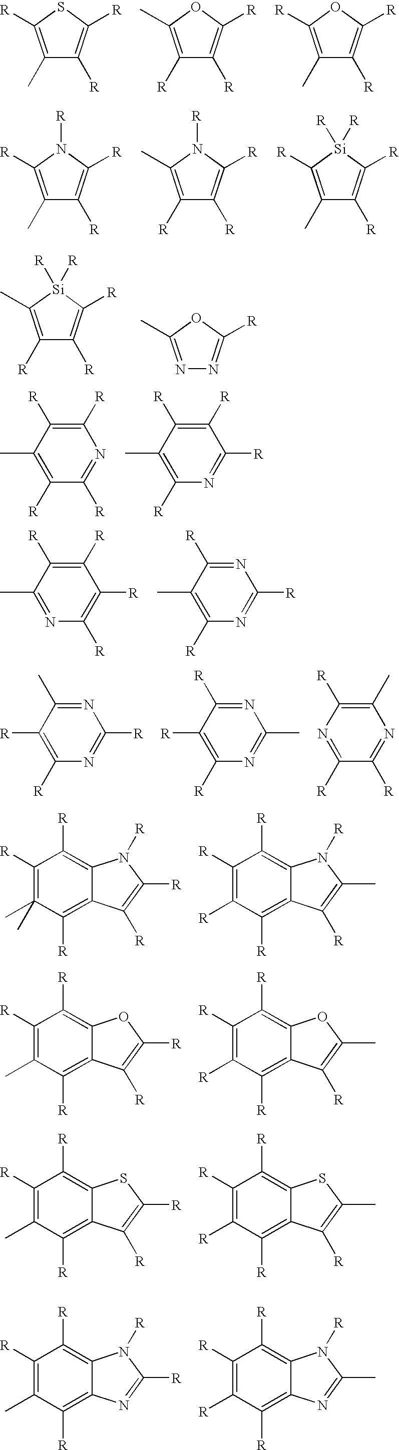

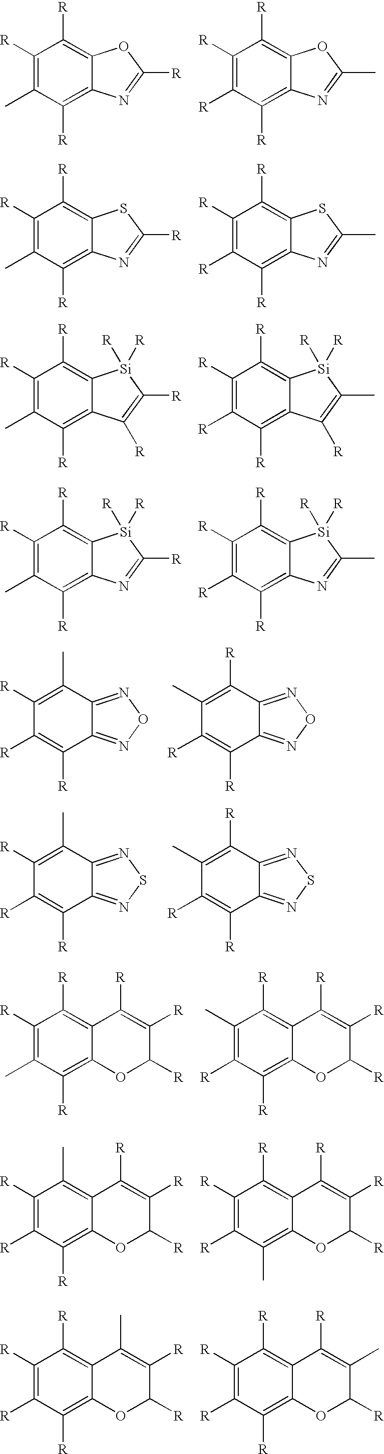

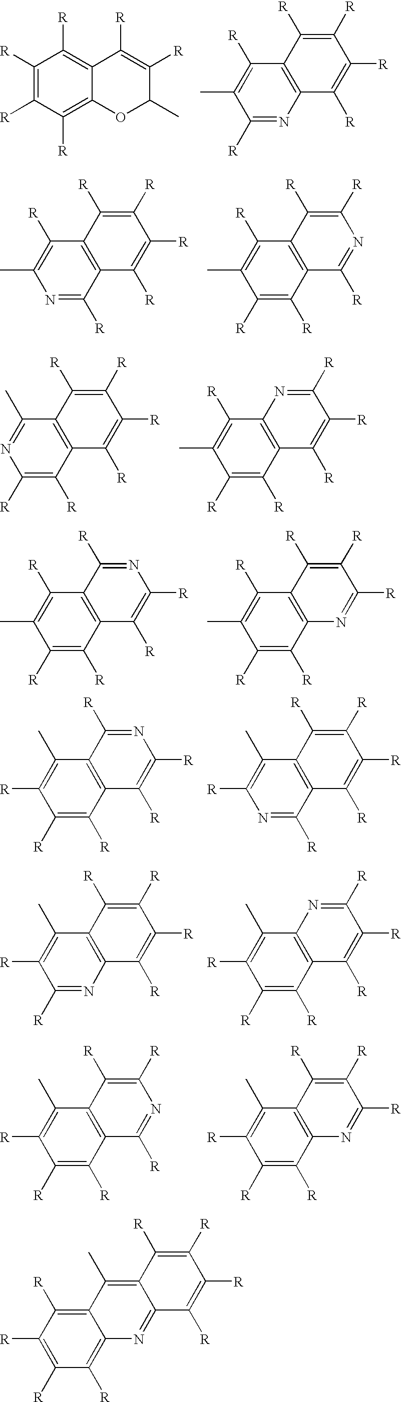

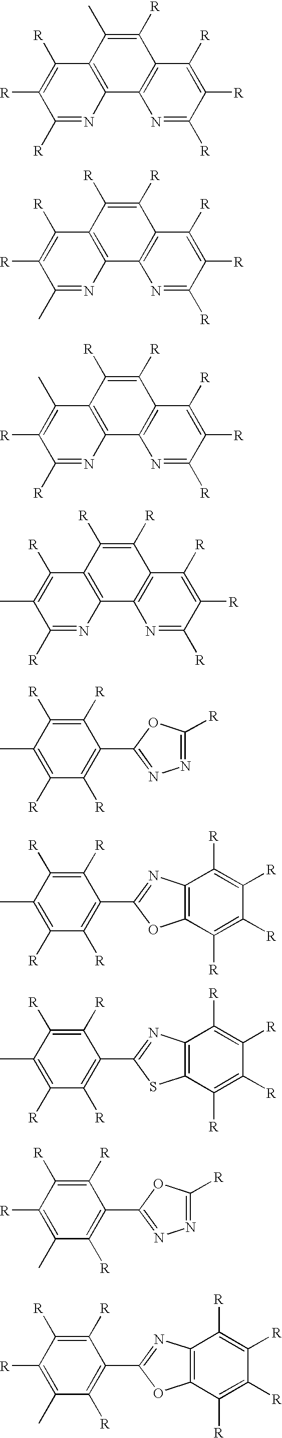

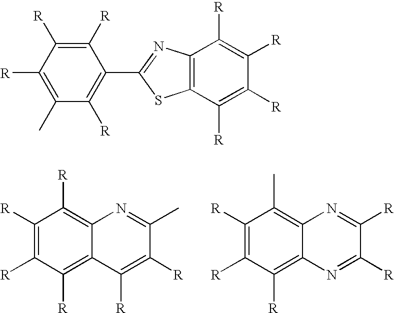

The monovalent aromatic amine group includes structures of the above-described formula (20) in which one of two bonds is capped by R.

The monovalent group derived from a heterocyclic ring coordinated metal complex includes structures in which one of two bonds in a divalent group having the above-described metal complex structure is capped by R.

Among the end groups, the aryl group having a formula weight of 90 or more has usually about from 6 to 60 carbon atoms. Here, the formula weight of the aryl group means a sum of products of the atomic weights and atomic numbers of respective elements in the chemical formula representing the aryl group.

The aryl group includes a phenyl group, a naphthyl group, an anthracenyl group, a group having a fluorene structure, a condensed ring compound group and the like.

Examples of the phenyl group capping an end include:

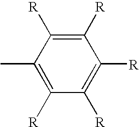

Examples of the naphthyl group capping an end include:

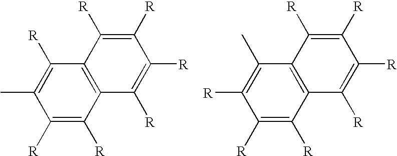

Examples of the anthracenyl group include:

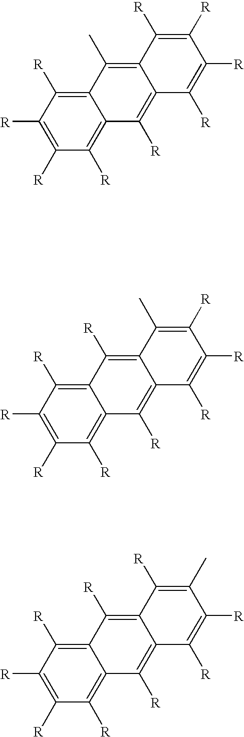

Examples of the group containing a fluorene structure include:

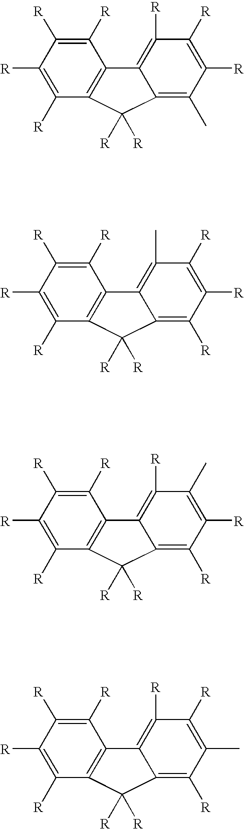

Examples of the condensed ring compound group include:

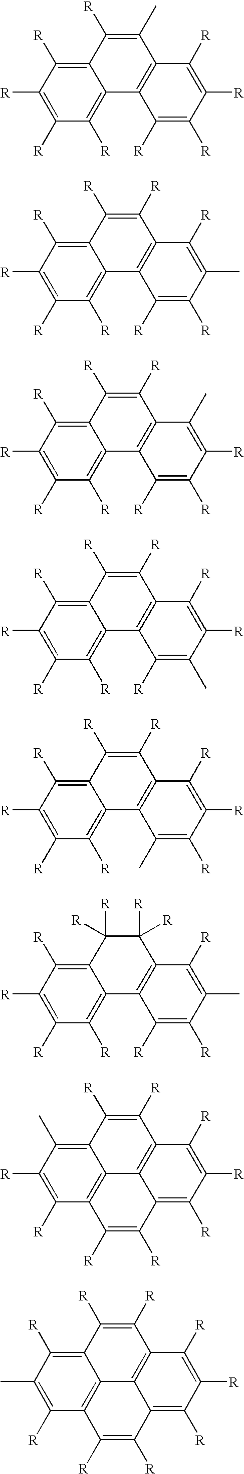

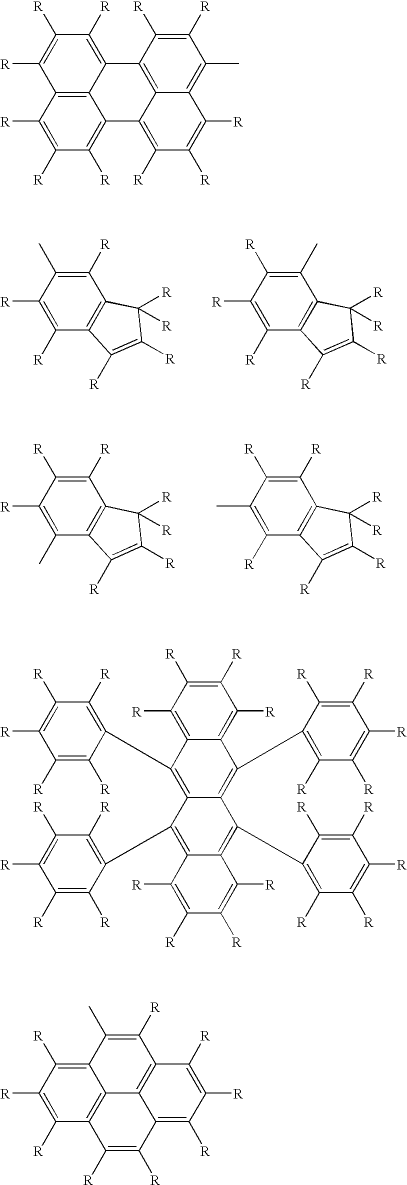

The end group enhancing charge injection property and charge transportability includes preferably monovalent heterocyclic groups, monovalent aromatic amine groups and condensed ring compound groups, and more preferably are monovalent heterocyclic groups and condensed ring compound groups.

The end group enhancing a light emission property includes preferably monovalent groups derived from a naphthyl group, anthracenyl group, condensed ring compound group, heterocyclic ring coordinated metal complex.

The end group having an effect of elongating time required for decrease in brilliance of a device includes preferably aryl groups having a substituent, and phenyl groups having 1 to 3 alkyl groups are preferable.

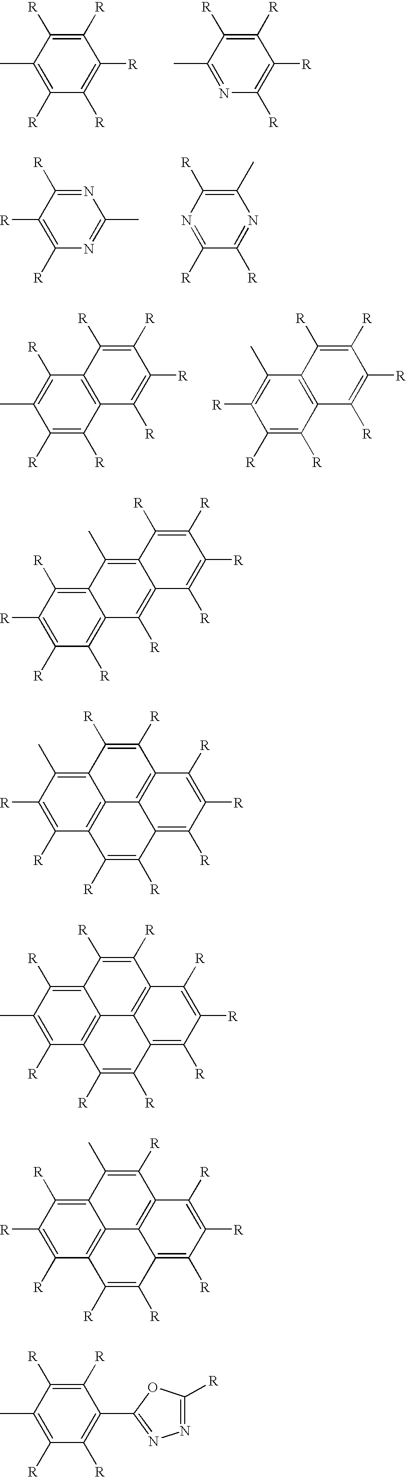

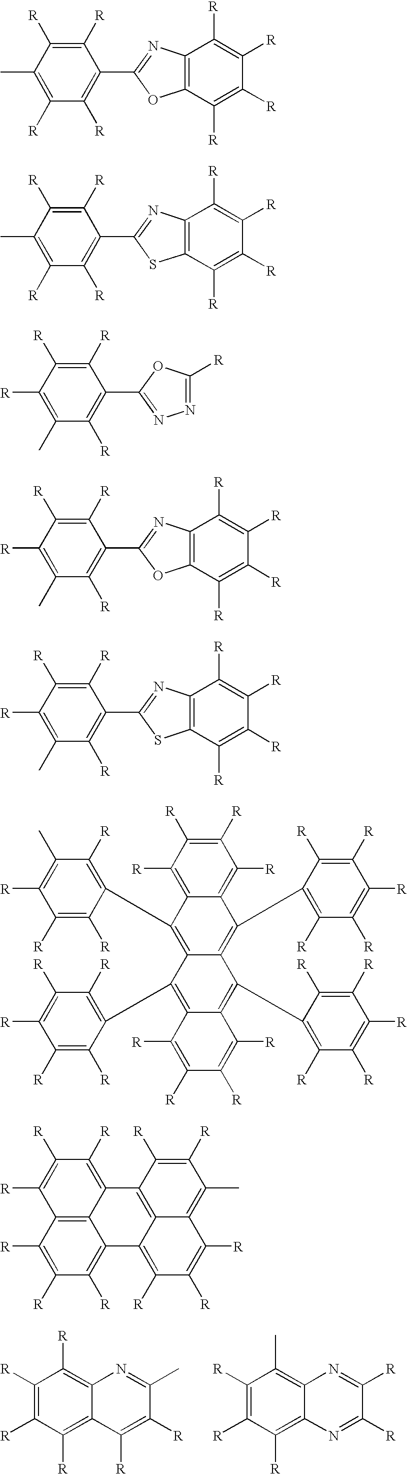

The end group having an effect of enhancing compatibility and mutual action between conjugated polymer compounds includes preferably aryl groups having a substituent. By use of phenyl groups substituted with an alkyl group having 6 or more carbon atoms, an anchor-like effect can be performed. The anchor effect means an effect that an end group plays an anchor-like role on an agglomerate of a polymer, to enhance a mutual action.

The group enhancing device properties preferably includes the following structures.

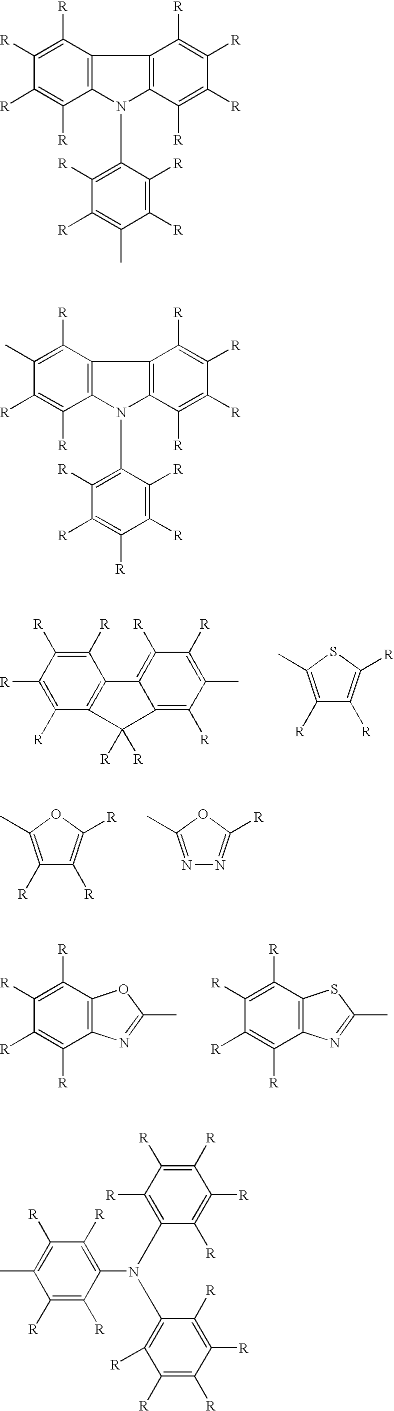

(As R in the formulae, the above-described examples for R are mentioned, and preferable are hydrogen, cyano group, alkyl group having 1 to 20 carbon atoms, alkoxy group, alkylthio group, aryl group having 6 to 18 carbon atoms, aryloxy group, and heterocyclic group having 4 to 14 carbon atoms.).

The group enhancing device properties preferably includes the following structures.

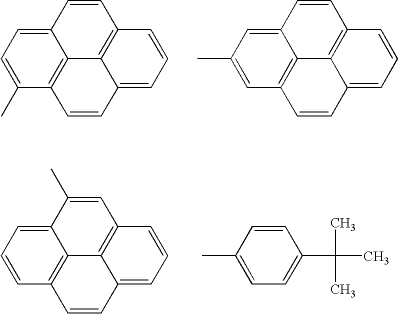

The preferable solvent for a conjugated polymer compound of the present invention includes chloroform, methylene chloride, dichloroethane, tetrahydrofuran, toluene, xylene, mesitylene, tetralin, decalin, n-butylbenzene and the like are illustrated. Depending on the structure and molecular weight of a conjugated polymer compound, the compound can be dissolved usually in an amount of 0.1 wt % or more in these solvents.

The conjugated copolymer compound of the present invention shows a fluorescence quantum yield of preferably 30% or more, more preferably 50% or more, further preferably 60% or more, from the standpoints of fluorescence intensity, device properties and the like.

One of properties desired for a conjugated polymer compound for polymer LED is electron injection property. Electron injection property generally depends on a value of the lowest unoccupied molecular orbital (LUMO) of a conjugated polymer compound, and when the value of the absolute value of LUMO is higher; electron injection property is more excellent. The absolute value of LUMO is preferably 2.5 eV or more, more preferably 2.7 eV or more, further preferably 2.8 eV or more.

When the conjugated polymer compound of the present invention is used in a polymer LED and the like, its purity exerts an influence on performances of a device such as a light emitting property and the like, therefore, it is preferable to purify a monomer before polymerization by a method such as distillation, sublimation purification, re-crystallization and the like before polymerization. It is preferable, after polymerization, to carry out a refinement treatment such as re-precipitation purification, chromatographic fractionation and the like. Among conjugated polymer compounds of the present invention, those produced by the method of polymerization with a nickel zerovalent complex are preferable from the standpoints of device properties such as life, light emission initiation voltage, current density, voltage increase in driving and the like, or heat resistance and the like, of a polymer LED.

Compounds in which the substituent participating in condensation polymerization in raw materials of a conjugated polymer compound of the present invention is halogen are obtained by synthesizing compounds having a structure in which Yᵣ₁, Yₛ₁, Yᵣ₂, Yₛ₂, Yₜ, Yᵤ, Yᵥ, Yₜ₁, Yᵤ₁, Yᵥ₁, Rₜ₂, Yᵤ₂ and Yᵥ₂ in the formulae (38), (39), (28), (29), (40), (41), (42), (31), (32), (33), (34), (43), (44) and (45) are substituted by a hydrogen atom, using, for example, a coupling reaction, ring-closing reaction and the like, then, halogenating them with various halogenation reagents such as, for example, chlorine, bromine, iodine, N-chlorosuccinimide, N-bromosuccinimide, benzyltrimethylammmonium tribromide and the like.

The substituent participating in condensation polymerization in raw materials of a conjugated polymer compound of the present invention is preferably a halogen, and from the standpoint of attaining higher molecular weight and from the standpoint of easiness of purification after completion of the reaction, the halogen is preferably bromine.

Compounds in which the substituent participating in condensation polymerization in raw materials of a conjugated polymer compound of the present invention is an alkyl sulfonate group, aryl sulfonate group or aryl alkyl sulfonate group are obtained, for example, by subjecting compounds having a functional group which can be derived into a hydroxyl group such as an alkoxy group and the like to a coupling reaction, ring-closing reaction and the like to synthesize compounds in which Yᵣ₁, Yₛ₁, Yᵣ₂, Yₛ₂, Yₜ, Yᵤ, Yᵥ, Yₜ₁, Yᵤ₁, Yᵥ₁, Yₜ₂, Yᵤ₂ and Yᵥ₂ in the formulae (38), (39), (28), (29), (40), (41), (42), (31), (32), (33), (34), (43), (44) and (45) are substituted by a functional group which can be derived into a hydroxyl group such as an alkoxy group and the like, then, synthesizing compounds in which Yᵣ₁, Yₛ₁, Yᵣ₂, Yₛ₂, Yₜ, Yᵤ, Yᵥ, Yₜ₁, Yᵤ₁, Yᵥ₁, Yₜ₂, Yᵤ₂ and Yᵥ₂ are substituted by a hydroxyl group by various reactions such as a reaction using a dealkylation reagent with, for example, boron tribromide and the like, then, sulfonylating a hydroxyl group with, for example, various sulfonyl chloride, sulfonic anhydride and the like. Compounds in which the substituent participating in condensation polymerization in raw materials of a conjugated polymer compound of the present invention is a boric acid group or borate group are obtained by synthesizing compounds in which Yᵣ₁, Yₛ₁, Yᵣ₂, Yₛ₂, Yₜ, Yᵤ, Yᵥ, Yₜ₁, Yᵤ₁, Yᵥ₁, Yₜ₂, Yᵤ₂ and Yᵥ₂ in the formulae (38), (39), (28), (29), (40), (41), (42), (31), (32), (33), (34), (43), (44) and (45) are substituted by a halogen atom, by the above-described methods, then, allowing alkyllithium, metal magnesium and the like to act on, further, forming boric acid with trimethyl borate, to convert the halogen atom into a boric acid group, and after the boric acid formation, allowing alcohol to act on thereby attaining borate formation. Further, compounds in which Yᵣ₁, Yₛ₁, Yᵣ₂, Yₛ₂, Yₜ, Yᵤ, Yᵥ, Yₜ₁, Yᵤ₁, Yᵥ₁, Yₜ₂, Yᵤ₂ and Yᵥ₂ in the formulae (38), (39), (28), (29), (40), (41), (42), (31), (32), (33), (34), (43), (44) and (45) are substituted by a halogen, trifluoromethane sulfonate group and the like are synthesized by the above-described methods, then, borate formation is carried out by methods described in non-patent literature (Journal of Organic Chemistry, 11995, 60, 7508-7510, Tetrahedron

Letters, 1997, 28 (19), 3447-3450) and the like. Among the conjugated polymer compounds of the present invention, those produced by the method of polymerization with a nickel zerovalent complex are preferable from the standpoint of a life property.

Next, use of the conjugated polymer compound of the present invention will be illustrated.

The conjugated polymer compound of the present invention usually emits fluorescence or phosphorescence in solid state and can be used as a polymer light emitter (light emitting material of high molecular weight).

The conjugated polymer compound has an excellent charge transporting ability, and can be suitably used as a polymer LED material or charge transporting material. The polymer LED using this polymer light emitter is a high performance polymer LED which can be driven at low voltage with high efficiency. Therefore, the polymer LED can be preferably used for back light of liquid crystal displays, curved or plane light source for illumination, segment type display, flat panel display of dot matrix, and the like.

The conjugated polymer compound of the present invention can also be used as a coloring matter for laser, a material for organic solar battery, an organic semiconductor for organic transistors, and a material for conductive thin films such as electrically conductive thin films, organic semiconductor thin films and the like.

Further, it can also be used as a light emitting thin film material which emits fluorescence or phosphorescence.

Next, use of the compound of the present invention will be illustrated.

The compound of the above-described formula (14) can be used as a LED material or charge transporting material.

Next, the polymer LED of the present invention will be illustrated.

The polymer LED of the present invention is characterized in that an organic layer is present between electrodes composed of an anode and a cathode and the organic layer contains a conjugated polymer compound of the present invention.

The organic layer (layer containing an organic substance) may be any of a light emitting layer, hole transporting layer, electron transporting layer and the like, and it is preferable that the organic layer is a light emitting layer.

Here, the light emitting layer means a layer having a function of light emission, the hole transporting layer means a layer having a function of transporting holes, and the electron transporting layer means a layer having a function of transporting electrons. The electron transporting layer and the hole transporting layer are generically called a charge transporting layer. Two or more light emitting layers, two or more hole transporting layers and two or more electron transporting layers may be used each individually.

When the organic layer is a light emitting layer, the light emitting layer as an organic layer may further contain a hole transporting material, electron transporting material or light emitting material. Here, the light emitting material means a material showing fluorescence or phosphorescence.

When the conjugated polymer compound of the present invention and a hole transporting material are mixed, the mixing ratio of the hole transporting material based on the whole mixtures is 1 wt % to 80 wt %, preferably 5 wt % to 60 wt %. When the polymer material of the present invention and an electron transporting material are mixed, the mixing ratio of the electron transporting material based on the whole mixtures is 1 wt % to 80 wt %, preferably 5 wt % to 60 wt %. Further, when the polymer compound and light emitting material of the present invention are mixed, the mixing ratio of the light emitting material based on the whole mixtures is 1 wt % to 80 wt %, preferably 5 wt % to 60 wt %. When the conjugated polymer compound of the present invention, a light emitting material, a hole transporting material and/or an electron transporting material are mixed, the mixing ratio of the light emitting material based on the whole mixtures is 1 wt % to 50 wt %, preferably 5 wt % to 40 wt %, the ratio of the sum of the hole transporting material and the electron transporting material is 1 wt % to 50 wt %, preferably 5 wt % to 40 wt %, and the content of the conjugated polymer compound of the present invention is 99 wt % to 20 wt %.

As the hole transporting material, electron transporting material and light emitting material to be mixed, known low molecular weight compounds, triplet light emitting complexes or conjugated polymer compounds can be used, and conjugated polymer compounds are preferably used. Examples of the hole transporting material, electron transporting material and light emitting material in the conjugated polymer compound include polyfluorene, derivatives and copolymers thereof, polyarylene, derivatives and copolymers thereof, polyarylenevinylene, derivatives and copolymers thereof, and (co)polymers of aromatic amines and its derivatives, disclosed in WO99/13692, WO99/48160, GB2340304A, WO00/53656, WO01/19834, WO00/55927, GB2348316, WO00/46321, WO00/06665, WO99/54943, WO99/54385, U.S. Pat. No. 5,77,7070, WO98/06773, WO97/05184, WO00/35987, WO00/53655, WO01/34722, WO99/24526, WO00/22027, WO00/22026, WO98/27136, U.S. Pat. No. 573,636, WO98/21262, U.S. Pat. No. 5,741,921, WO97/09394, WO96/29356, WO96/10617, EP0707020, WO95/07955, JP-A No. 2001-181618, JP-A No. 2001-123156, JP-A No. 2001-3045, JP-A No. 2000-351967, JP-A No. 2000-303066, JP-A No. 2000-299189, JP-A No. 2000-252065, JP-A No. 2000-136379, JP-A No. 2000-104057, JP-A No. 2000-80167, JP-A No. 10-324870, JP-A No. 10-114891, JP-A No. 9-111233, JP-A No. 9-45478 and the like.

As the fluorescent material of lower molecular weight, there can be used, for example, naphthalene derivatives, anthracene or its derivatives, perylene or its derivatives, and polymethine, xanthene, coumarin and cyanine coloring matters, metal complexes of 8-hydroxyquinoline or its derivatives, aromatic amines, tetraphenylcyclopentadiene or its derivatives, or tetraphenylbutadiene or its derivatives, and the like.

Known compounds such as those described in, for example, JP-A Nos. 57-51781, 59-194393, and the like can be used.

The triplet light emitting complex includes for example, Ir(ppy)₃, Btp₂Ir(acac) containing iridium as a central metal, PtOEP containing platinum as a central metal, Eu(TTA)₃phen containing europium as a central metal, and the like are mentioned.

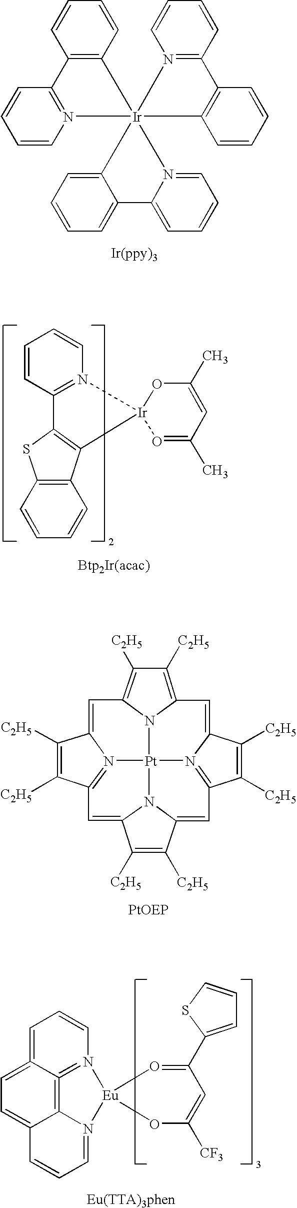

The triplet light emitting complex is described, for example, in Nature, (1998), 395, 151, Appl. Phys. Lett. (1999), 75(1), 4, Proc. SPIE-Int. Soc. Opt. Eng. (2001), 4105 (Organic Light-Emitting Materials and Devices IV), 119, J. Am. Chem. Soc., (2001), 123, 4304, Appl. Phys. Lett., (1997), 71(18), 2596, Syn. Met., (1998), 94(1), 103, Syn. Met., (1999), 99(2), 1361, Adv. Mater., (1999), 11(10), 852, Jpn. J. Appl. Phys., 34, 1883 (1995) and the like.

The conjugated polymer compound of the present invention can be excellent in heat resistance, by having a structure of the formula (1).

In the conjugated polymer compound of the present invention, the glass transition temperature is preferably 130° C. or higher, more preferably 150° C. or higher, further preferably 160° C. or higher.

The composition of the present invention contains at least one material selected from hole transporting materials, electron transporting materials and light emitting materials, and a conjugated polymer compound of the present invention, and can be used as a light emitting material or transporting material.

The content ratio of at least one material selected from hole transporting materials, electron transporting materials and light emitting materials to a conjugated polymer compound of the present invention may be determined depending on its use, and in the case of use of a light emitting material, the same content ratio as in the above-described light emitting layer is preferable.

As another embodiment of the present invention, a polymer composition containing two or more conjugated polymer compounds of the present invention is illustrated.

Specifically, a polymer composition containing two or more conjugated polymer compounds containing a repeating unit of the above-described formula (2) or (3) in which the total amount of the conjugated polymer compounds is 50 wt % or more based on the total amount is preferable because of excellent light emission efficiency, characteristic of life and the like when used as a light emitting material of polymer LED. More preferably, the total amount of the conjugated polymer compounds is 70 wt % or more based on the total amount.

The polymer composition of the present invention can enhance device properties such as life and the like more than in the case of use of a conjugated polymer compound singly in polymer LED.

When the conjugated polymer compound of the present invention is used in the form of polymer composition, the repeating unit of the above-described formula (2) or (3) is preferably selected from a repeating unit of the above-described formula (6) or a repeating unit of the formula (7), and a case of a repeating unit of the formula (6) is more preferable, and a case of a repeating unit of the formula (6) in which a and b are 0 is further preferable, from the standpoint of solubility in an organic solvent and from the standpoints of device properties such as light emission efficiency, life property and the like. The repeating unit of the above-described formula (20) is further preferably a repeating unit of the above-described formula 134 or a repeating unit of the above-described formula 137.

The polymer composition of the present invention has a polystyrene-reduced number-average molecular weight of usually about from 10³ to 10⁸, preferably 10⁴ to 10⁶. The polystyrene-reduced weight-average molecular weight is usually about from 10³ to 10⁸, and from the standpoint of film formability and from the standpoint of efficiency when made into a device, preferably 5×10⁴ to 5×10⁶, further preferably 10⁵ to 5×10⁶. Here, the average molecular weight of a polymer composition means a value obtained by GPC-analysis of a composition obtained by mixing two or more conjugated polymer compounds.

The thickness of a light emitting layer of the polymer LED of the present invention shows an optimum value depending on the material to be used and may be advantageously selected so as to give suitable driving voltage and light emission efficiency, and it is, for example, 1 nm to 1 μm, preferably 2 nm to 500 nm, further preferably 5 nm to 200 nm.

The method for forming a light emitting layer includes a method of film formation from a solution. The film formation method from a solution includes application methods such as a spin coat method, casting method, micro gravure coat method, gravure coat method, bar coat method, roll coat method, wire bar coat method, dip coat method, spray coat method, screen printing method, flexo printing method, offset printing method, inkjet printing method and the like. Printing methods such as a screen printing method, flexo printing method, offset printing method, inkjet printing method and the like are preferable since pattern formation and multicolor separate painting are easier.

As the solution (ink composition) used in printing methods, it is advantageous that at least one of conjugated polymer compounds of the present invention be advantageously contained, and in addition to the conjugated polymer compound of the present invention, additives such as a hole transporting material, electron transporting material, light emitting material, solvent, stabilizer and the like may be contained.

The ratio of a conjugated polymer compound of the present invention in the ink composition is usually from 20 wt % to 100 wt %, preferably 40 wt % to 100 wt % based on the total weight of the composition excluding a solvent.

The ratio of a solvent when the ink composition contains a solvent is from 1 wt % to 99.9 wt % based on the total weight of the composition.

Though the viscosity of an ink composition depends on a printing method, when an ink composition passes through a discharge apparatus such as in an inkjet print method and the like, the viscosity at 25° C. is preferably in a range of 1 to 20 mPa·s, more preferably in a range of 5 to 20 mPa·s, for preventing clogging and curving in flying in discharging.

The solution of the present invention may contain additives for regulating viscosity and/or surface tension in addition to the conjugated polymer compound of the present invention. As the additive, a conjugated polymer compound (thickening agent) having high molecular weight for enhancing viscosity and a poor solvent, a compound of low molecular weight for lowering viscosity, a surfactant for decreasing surface tension, and the like may be appropriately combined and used.

As the above-described conjugated polymer compound having high molecular weight, a compound which is soluble in the same solvent as for the conjugated polymer compound of the present invention and which does not disturb light emission and charge transportation may be used. For example, polystyrene of high molecular weight, polymethyl methacrylate, conjugated polymer compounds of the present invention having larger molecular weights, and the like can be used. The weight-average molecular weight is preferably 500000 or more, more preferably 1000000 or more.

It is also possible to use a poor solvent as a thickening agent. Namely, by adding a small amount of poor solvent for solid components in the solution, viscosity can be enhanced. When a poor solvent is added for this purpose, the kind and addition amount of the solvent may be advantageously selected within a range not causing deposition of solid components in the solution. When stability in preservation is taken into consideration, the amount of a poor solvent is preferably 50 wt % or less based on the whole solution.

The solution of the present invention may contain an antioxidant in addition to the conjugated polymer compound of the present invention for improving preservation stability. As the antioxidant, a compound which is soluble in the same solvent as for the conjugated polymer compound of the present invention and which does not disturb light emission and charge transportation is permissible, and examples are phenol-type antioxidants, phosphorus-based antioxidants and the like.

The solvent used in film formation from a solution includes compounds which can dissolve or uniformly disperse a conjugated polymer compound of the present invention. Examples of the solvent include chlorine-based solvents such as chloroform, methylene chloride, 1,2-dichloroethane, 1,1,2-trichloroethane, chlorobenzene, o-dichlorobenzene and the like, ether solvents such as tetrahydrofuran, dioxane and the like, aromatic hydrocarbon solvents such as toluene, xylene and the like, aliphatic hydrocarbon solvents such as cyclohexane, methylcyclohexane, n-pentane, n-hexane, n-heptane, n-octane, n-nonane, n-decane and the like, ketone solvents such as acetone, methyl ethyl ketone, cyclohexanone and the like, ester solvents such as ethyl acetate, butyl acetate, ethylcellosolve acetate and the like, polyhydric alcohols such as ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerin, 1,2-hexanediol and the like and derivatives thereof, alcohol solvents such as methanol, ethanol, propanol, isopropanol, cyclohexanol and the like, sulfoxide solvents such as dimethyl sulfoxide and the like, amide solvents such as N-methyl-2-pyrrolidone, N,N-dimethylformamide and the like. These organic solvents can be used singly or in combination of two or more. Among the above-described solvents, at least one organic solvent having a structure containing at least one benzene ring and having a melting point of 0° C. or lower and a boiling point of 100° C. or higher is preferably contained.

The solvent preferably includes aromatic hydrocarbon solvents, aliphatic hydrocarbon solvents, ester solvents and ketone solvents from the standpoints of solubility in organic solvents, uniformity in film formation, viscosity property and the like, and toluene, xylene, ethylbenzene, diethylbenzene, trimethylbenzene, n-propylbenzene, i-propylbenzene, n-butylbenzene, i-butylbenzene, s-butylbenzene, anisole, ethoxybenzene, 1-methylnaphthalene, cyclohexane, cyclohexanone, cyclohexylbenzene, bicyclohexyl, cyclohexenylcyclohexanone, n-heptylcyclohexane, n-hexylcyclohexanone, 2-propylcyclohexanone, 2-heptanone, 3-heptanone, 4-heptanone, 2-octanone, 2-nonanone, 2-decanone, tetralin, dicyclohexylketone, cyclohexanone, phenylhexane and decalin are preferable, and it is more preferable that at least one of xylene, anisole, cyclohexylbenzene, bicyclohexyl, cyclohexanone, phenylhexane and decalin is contained.

The number of solvents in a solution is preferably 2 or more, more preferably 2 to 3, further preferably 2, from the standpoint of film formability and from the standpoints of device properties and the like.

When two solvents are contained in a solution, one of them may be solid at 25° C. From the standpoint of film formability, it is preferable that one solvent has a boiling point of 180° C. or higher, and another solvent has a boiling point of 180° C. or lower, and it is more preferable that one solvent has a boiling point of 200° C. or higher, and another solvent has a boiling point of 180° C. or lower. From the standpoint of viscosity, it is preferable that a conjugated polymer compound is dissolved in an amount of 1 wt % or more at 60° C. in both solvents, and it is preferable that one of two solvents dissolves a conjugated polymer compound in an amount of 1 wt % or more at 25° C.

When three solvents are contained in a solution, one or two solvents may be solid at 25° C. From the standpoint of film formability, it is preferable that at least one of three solvents has a boiling point of 180° C. or higher and at least one solvent has a boiling point of 180° C. or lower, and it is more preferable that at least one of three solvents has a boiling point of 200° C. or higher and 300° C. or lower and at least one solvent has a boiling point of 180° C. or lower. From the standpoint of viscosity, it is preferable that a conjugated polymer compound is dissolved in an amount of 1 wt % or more at 60° C. in two solvents among three solvents, and it is preferable that a conjugated polymer compound is dissolved in an amount of 1 wt % or more at 25° C. in one of three solvents.

When two or more solvents are contained in a solution, the content of a solvent having highest boiling point is preferably from 40 to 90 wt %, more preferably 50 to 90 wt % based on the weight of all solvents in the solution from the standpoints of viscosity and film formability.

As the solution of the present invention, mentioned are, for example, a solution composed of anisole and bicyclohexyl, a solution composed of anisole and cyclohexylbenzene, a solution composed of xylene and bicyclohexyl, and a solution composed of xylene and cyclohexylbenzene, from the standpoints of viscosity and film formability.

From the standpoint of solubility of a conjugated polymer compound in a solvent, a difference between the solubility parameter of a solvent and the solubility parameter of a conjugated polymer compound is preferably 10 or less, more preferably 7 or less.

The solubility parameter of a solvent and the solubility parameter of a conjugated polymer compound can be measured by a method described in “Solvent Handbook (Kodansha, 1976)”.

The conjugated polymer compounds of the present invention may be contained singly or in combination of two or more in a solution, and a conjugated polymer compound other than the conjugated polymer compound of the present invention may also be contained in a range not deteriorating device properties and the like.

The solution of the present invention may contain water, metal and its salt in an amount of from 1 to 1000 ppm. Specifically mentioned as the metal are lithium, sodium, calcium, potassium, iron, copper, nickel, aluminum, zinc, chromium, manganese, cobalt, platinum, iridium and the like. Further, silicon, phosphorus, fluorine, chlorine or bromine may be contained in an amount of from 1 to 1000 ppm.

Using the solution of the present invention, a thin film can be formed by a spin coat method, casting method, micro gravure coat method, gravure coat method, bar coat method, roll coat method, wire bar coat method, dip coat method, spray coat method, screen printing method, flexo printing method, offset printing method, inkjet printing method and the like. Particularly, the solution of the present invention is preferably used for film formation by a screen printing method, flexo printing method, offset printing method, inkjet printing method, and more preferably used for film formation by an inkjet method.

When a thin film is formed using the solution of the present invention, a conjugated polymer compound contained in the solution has high glass transition temperature, therefore, baking at temperatures of 100° C. or higher is possible, and even if baking is carried out at a temperature of 130° C., decrease in device properties is very small. Depending on the kind of a conjugated polymer compound, it is also possible to carry out baking at temperatures of 160° C. or higher.

As the thin film which can be formed using the solution of the present invention, a light emitting thin film, electrically conductive thin film and organic semiconductor thin film are illustrated.

The light emitting thin film of the present invention shows a quantum yield of light emission of preferably 50% or more, more preferably 60% or more, further preferably 70% or more from the standpoints of the brilliance and light emission voltage of a device, and the like.

The electrically conductive thin film of the present invention has a surface resistance of 1 KO/□ or less. By doping a thin film with a Lewis acid, ionic compound and the like, electric conductivity can be enhanced. The surface resistance is preferably 100 KO/□ or less, further preferably 10 KO/□ or less.

In the organic semiconductor thin film of the present invention, one larger parameter of electron mobility or hole mobility is preferably 10⁻⁵ cm²/V/s or more. More preferably, it is 10⁻³ cm²/V/s or more, and further preferably 10⁻¹ cm²/V/s or more.

By forming the organic semiconductor thin film on a Si substrate carrying a gate electrode and an insulation film made of SiO₂ and the like formed thereon, and forming a source electrode and a drain electrode with Au and the like, an organic transistor can be obtained.

In the polymer light emitting device of the present invention, the maximum external quantum yield when a voltage of 3.5 V or more is applied between an anode and a cathode is preferably 1% or more, more preferably 1.5% or more from the standpoint of the brilliance of a device or the like.

The polymer light emitting device (hereinafter, referred to as polymer LED) of the present invention, includes polymer LED having an electron transporting layer provided between a cathode and a light emitting layer, polymer LED having a hole transporting layer provided between an anode and a light emitting layer, polymer LED having an electron transporting layer provided between a cathode and a light emitting layer and a hole transporting layer provided between an anode and a light emitting layer, and the like.

For example, the following structures a) to d) are illustrated.

a) anode/light emitting layer/cathode

b) anode/hole transporting layer/light emitting layer/cathode

c) anode/light emitting layer/electron transporting layer/cathode

d) anode/hole transporting layer/light emitting layer/electron transporting layer/cathode (wherein, / means adjacent lamination of layers, applicable also in the followings)

The polymer LED of the present invention includes also those in which a conjugated polymer compound of the present invention is contained in a hole transporting layer and/or electron transporting layer.

When the conjugated polymer compound of the present invention is used in a hole transporting layer, it is preferable that the conjugated polymer compound of the present invention is a conjugated polymer compound containing a hole transporting group, and examples thereof include copolymers with an aromatic amine, copolymers with stilbene, and the like.

When the conjugated polymer compound of the present invention is used in an electron transporting layer, it is preferable that the conjugated polymer compound of the present invention is a conjugated polymer compound containing an electron transporting group, and examples thereof include copolymers with oxadiazole, copolymers with triazole, copolymers with quinoline, copolymers with quinoxaline, copolymers with benzothiazole, and the like.

When the polymer LED of the present invention contains a hole transporting layer, examples of the hole transporting material to be used include polyvinylcarbazole or its derivatives, polysilane or its derivatives, polysiloxane derivatives having an aromatic amine on the side chain or main chain, pyrazoline derivatives, arylamine derivatives, stilbene derivatives, triphenyldiamine derivatives, polyaniline or its derivatives, polythiophene or its derivatives, polypyrrole or its derivatives, poly(p-phenylenevinylene) or its derivatives, poly(2,5-thienylenevinylene) or its derivatives, and the like.

Examples of the hole transporting material are those described in JP-A Nos. 63-70257, 63-175860, 2-135359, 2-135361, 2-209988, 3-37992 and 3-152184, and the like.

Among them, preferable as the hole transporting material used in a hole transporting layer are polymer hole transporting materials such as polyvinylcarbazole or its derivatives, polysilane or its derivatives, polysiloxane derivatives having an aromatic amine compound group on the side chain or main chain, polyaniline or its derivatives, polythiophene or its derivatives, poly(p-phenylenevinylene) or its derivatives, poly(2,5-thienylenevinylene) or its derivatives, and the like, and polyvinylcarbazole or its derivatives, polysilane or its derivatives, and polysiloxane derivatives having an aromatic amine on the side chain or main chain are further preferable.

Examples of the hole transporting material of low molecular weight include pyrazoline derivatives, arylamine derivatives, stilbene derivatives and triphenyldiamine derivatives. In the case of the hole transporting material of low molecular weight, it is preferably dispersed in a polymer binder in use.

The preferable polymer binder to be mixed is that which does not extremely disturb charge transportation, and those showing not strong absorption against visible ray are suitably used. Examples of the polymer binder include poly(N-vinylcarbazole), polyaniline or its derivatives, polythiophene or its derivatives, poly(p-phenylenevinylene) or its derivatives, poly(2,5-thienylenevinylene) or its derivatives, polycarbonate, polyacrylate, polymethyl acrylate, polymethyl methacrylate, polystyrene, polyvinyl chloride, polysiloxane and the like.

Polyvinylcarbazole or its derivatives can be obtained, for example, from a vinyl monomer by cation polymerization or radical polymerization.

The polysilane or its derivatives includes compounds described in Chem. Rev., vol. 89, p. 1359 (1989), GB Patent No. 2300196 publication, and the like. Also as the synthesis method, methods described in them can be used, and particularly, a Kipping method is suitably used.

In the polysiloxane or its derivatives, the siloxane skeleton structure shows little hole transportability, thus, those having a structure of the above-described hole transporting material of low molecular weight on the side chain or main chain are suitably used. Particularly, those having a hole transportable aromatic amine on the side chain or main chain are illustrated.

The film formation method of a hole transporting layer is not particularly restricted, and in the case of a hole transporting material of low molecular weight, a method of film formation from a mixed solution with a polymer binder is illustrated. In the case of a hole transporting material of high molecular weight, a method of film formation from a solution is illustrated.

As the solvent used for film formation from a solution, those which can dissolve or uniformly disperse a hole transporting material are preferable. Examples of the solvent include chlorine-based solvents such as chloroform, methylene chloride, 1,2-dichloroethane, 1,1,2-trichloroethane, chlorobenzene, o-dichlorobenzene and the like, ether solvents such as tetrahydrofuran, dioxane and the like, aromatic hydrocarbon solvents such as toluene, xylene and the like, aliphatic hydrocarbon solvents such as cyclohexane, methylcyclohexane, n-pentane, n-hexane, n-heptane, n-octane, n-nonane, n-decane and the like, ketone solvents such as acetone, methyl ethyl ketone, cyclohexanone and the like, ester solvents such as ethyl acetate, butyl acetate, ethylcellosolve acetate and the like, polyhydric alcohols such as ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerin, 1,2-hexanediol and the like and derivatives thereof, alcohol solvents such as methanol, ethanol propanol, isopropanol, cyclohexanol and the like, sulfoxide solvents such as dimethyl sulfoxide and the like, amide solvents such as N-methyl-2-pyrrolidone, N,N-dimethylformamide and the like. These organic solvents can be used singly or in combination of two or more.

The method for film formation from a solution includes application methods from a solution such as a spin coat method, casting method, micro gravure coat method, gravure coat method, bar coat method, roll coat method, wire bar coat method, dip coat method, spray coat method, screen printing method, flexo printing method, offset printing method, inkjet printing method and the like.

Regarding the thickness of a hole transporting layer, the optimum value varies depending on a material used, and it may be advantageously selected so that the driving voltage and light emission efficiency show suitable values, and a thickness at least causing no formation of pin holes is necessary, and when the thickness is too large, the driving voltage of a device increases undesirably. Therefore, the thickness of the hole transporting layer is, for example, from 1 nm to 1 μm, preferably 2 nm to 500 nm, further preferably 5 nm to 200 nm.

When the polymer LED of the present invention has an electron transporting layer, known materials can be used as the electron transporting material to be used, and examples are oxadiazole derivatives, anthraquinodimethane or its derivatives, benzoquinone or its derivatives, naphthoquinone or its derivatives, anthraquinone or its derivatives, tetracyanoanthraquinodimethane or its derivatives, fluorenone derivatives, diphenyldicyanoethylene or its derivatives, diphenoquinone derivatives, metal complexes of 8-hydroxyquinoline or its derivatives, polyquinoline or its derivatives, polyquinoxaline or its derivatives, polyfluorene or its derivatives, and the like.

Specifically, those described in JP-A Nos. 63-70257, 63-175860, 2-135359, 2-135361, 2-209988, 3-37992, 3-152184, and the like are illustrated.

Of them, oxadiazole derivatives, benzoquinone or its derivatives, anthraquinone or its derivatives, metal complexes of 8-hydroxyquinoline or its derivatives, polyquinoline or its derivatives, polyquinoxaline or its derivatives, polyfluorene or its derivatives are preferable, and 2-(4-biphenylyl)-5-(4-t-butylphenyl)-1,3,4-oxadiazole, benzouqinone, anthraquinone, tris(8-quinolinol)aluminum and polyquinoline are further preferable.

The film formation method of an electron transporting layer is not particularly restricted, and in the case of an electron transporting material of low molecular weight, examples are a vacuum vapor-deposition method from powder, film formation methods from solution or melted conditions, and in the case of an electron transporting material of high molecular weight, film formation methods from solution or melted condition are illustrated, respectively. In film formation from solution or melted condition, the above-described polymer binder may be used together.

As the solvent used for film formation from a solution, compounds which can dissolve or uniformly disperse an electron transporting material and/or polymer binder are preferable. Examples of the solvent include solvents having chlorine atom such as chloroform, methylene chloride, 1,2-dichloroethane, 1,1,2-trichloroethane, chlorobenzene, o-dichlorobenzene and the like, ether solvents such as tetrahydrofuran, dioxane and the like, aromatic hydrocarbon solvents such as toluene, xylene and the like, aliphatic hydrocarbon solvents such as cyclohexane, methylcyclohexane, n-pentane, n-hexane, n-heptane, n-octane, n-nonane, n-decane and the like, ketone solvents such as acetone, methyl ethyl ketone, cyclohexanone and the like, ester solvents such as ethyl acetate, butyl acetate, ethylcellosolve acetate and the like, polyhydric alcohols such as ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerin, 1,2-hexanediol and the like and derivatives thereof, alcohol solvents such as methanol, ethanol, propanol, isopropanol, cyclohexanol and the like, sulfoxide solvents such as dimethyl sulfoxide and the like, amide solvents such as N-methyl-2-pyrrolidone, N,N-dimethylformamide and the like. These organic solvents can be used singly or in combination of two or more.

The film formation method from solution or melted condition includes application methods such as a spin coat method, casting method, micro gravure coat method, gravure coat method, bar coat method, roll coat method, wire bar coat method, dip coat method, spray coat method, screen printing method, flexo printing method, offset printing method, inkjet printing method and the like.

The conjugated polymer compound of the present invention can also be used as a polymer electric field effect transistor. In the structure of a polymer electric field effect transistor, it may be usually advantageous that a source electrode and a drain electrode are placed next to an active layer made of a polymer, further, a gate electrode is provided sandwiching an insulating layer next to the active layer.

The polymer electric field effect transistor is usually formed on a supporting substrate. The material of the supporting substrate is not particularly restricted providing it does not disturb a property as an electric field effect transistor, and a glass substrate, flexile film substrate and plastic substrate can also be used.

The polymer electric field effect transistor can be produced by known methods, for example, a method described in JP-A No. 5-110069.

In forming an active layer, it is very advantageous and preferable to use a polymer soluble in organic solvents from the standpoint of production. The film formation method from a solution prepared by dissolving a polymer in an organic solvent includes application methods such as a spin coat method, casting method, micro gravure coat method, gravure coat method, bar coat method, roll coat method, wire bar coat method, dip coat method, spray coat method, screen printing method, flexo printing method, offset printing method, inkjet printing method and the like.

Preferable is an encapsulated polymer electric field effect transistor obtained by producing a polymer electric field effect transistor and then, encapsulating this. By this, the polymer electric field effect transistor is blocked from atmospheric air, and decrease in the property of the polymer electric field effect transistor can be suppressed.

The encapsulating method includes a method of covering with a UV hardening resin, thermosetting resin, inorganic SiONx film and the like, a method of pasting a glass plate or film with a UV hardening resin, thermosetting resin and the like. It is preferable that a process from manufacturing of a polymer electric field effect transistor until encapsulation is carried out without exposing to atmospheric air (for example, in a dried nitrogen atmosphere, in vacuum, and the like), for effectively performing blocking from atmospheric air.

Regarding the thickness of an electron transporting layer, the optimum value varies depending on a material to be used, and it may be advantageously selected so that the driving voltage and light emission efficiency show suitable values, and a thickness at least causing no formation of pin holes is necessary, and when the thickness is too large, the driving voltage of a device increases undesirably. Therefore, the thickness of the electron transporting layer is, for example, from 1 nm to 1 μm, preferably 2 nm to 500 nm, further preferably 5 nm to 200 nm.

Among charge transporting layers placed next to an electrode, those having a function of improving charge injecting efficiency from an electrode and having an effect of lowering the driving voltage of a device are, in particularly, called generally a charge injection layer (hole injection layer, electron injection layer).

Further, for improving close adherence with an electrode or improving charge injection from an electron, the above-described charge injection layer or an insulation layer having a thickness of 2 nm or less may be placed next to the electrode, alternatively, for improving close adherence of an interface or preventing mixing, a thin buffer layer may be inserted into an interface of a charge transporting layer and a light emitting layer.

The order and number of layers to be laminated, and thickness of each layer can be appropriately determined in view of light emission efficiency and life of device.

In the present invention, as the polymer LED carrying a provided charge injection layer (electron injection layer, hole injection layer), mentioned are polymer LED having a charge injection layer placed next to a cathode and polymer LED having a charge injection layer next to an anode.

For example, the following structures e) to p) are specifically mentioned.

e) anode/hole injection layer/light emitting layer/cathode

f) anode/light emitting layer/electron injection layer/cathode

g) anode/hole injection layer/light emitting layer/electron injection layer/cathode

h) anode/hole injection layer/hole transporting layer/light emitting layer/cathode

i) anode/hole injection layer/light emitting layer/electron injection layer/cathode

j) anode/hole injection layer/hole transporting layer/light emitting layer/electron injection layer/cathode

k) anode/hole injection layer/light emitting layer/electron transporting layer/cathode

l) anode/light emitting layer/electron transporting layer/electron injection layer/cathode

m) anode/hole injection layer/light emitting layer/electron transporting layer/electron injection layer/cathode

n) anode/hole injection layer/hole transporting layer/light emitting layer/electron transporting layer/cathode

o) anode/hole transporting layer/light emitting layer/electron transporting layer/electron injection layer/cathode

p) anode/hole injection layer/hole transporting layer/light emitting layer/electron transporting layer/electron injection layer/cathode

The polymer LED of the present invention includes also those in which a conjugated polymer compound of the present invention is contained in a hole transporting layer and/or electron transporting layer, as described above.

The polymer LED of the present invention includes also those in which a conjugated polymer compound of the present invention is contained in a hole injection layer and/or electron injection layer. When a conjugated polymer compound of the present invention is used in a hole injection layer, it is preferable that the compound is used simultaneously with an electron receptive compound. When a conjugated polymer compound of the present invention is used in an electron transporting layer, it is preferable that the compound is used simultaneously with an electron donating compound. Here, for simultaneous use, there are methods such as mixing, copolymerization, introduction as a side chain, and the like.

Examples of the electric charge injection layer include a layer containing an electric conductive polymer, a layer provided between an anode and a hole transporting layer and containing a material having ionization potential of a value between an anode material and a hole transporting material contained in a hole transporting layer, a layer provided between an anode and an electron transporting layer and containing a material having electron affinity of a value between a cathode material and an electron transporting material contained in an electron transporting layer, and the like.

When the above-described charge injection layer contains an electric conductive polymer, electric conductivity of the electric conductive polymer is preferably 10⁻⁵ S/cm or more and 10³ or less, and for decreasing leak current between light emission picture elements, more preferably 10⁻⁵ S/cm or more and 10² or less, further preferably 10⁻⁵ S/cm or more and 10¹ or less.

When the above-described charge injection layer contains an electric conductive polymer, electric conductivity of the electric conductive polymer is preferably 10⁻⁵ S/cm or more and 10³ or less, and for decreasing leak current between light emission picture elements, more preferably 10⁻⁵ S/cm or more and 10² or less, further preferably 10⁻⁵ S/cm or more and 10¹ or less.

Usually, for controlling the electric conductivity of the electric conductive polymer to 10⁻⁵ S/cm or more and 10³ or less, the electric conductive polymer is doped with a suitable amount of electrons.

As the kind of ions to be doped, an anion is used in a hole injection layer and a cation is used in en electron injection layer. Examples of the anion include a polystyrenesulfonic ion, alkylbenzenesulfonic ion, camphorsulfonic ion and the like, and examples of the cation include a lithium ion, sodium ion, potassium ion, tetrabutylammonium ion and the like.

The thickness of the charge injection layer is, for example, from 1 nm to 100 nm, preferably 2 nm to 50 nm.

The material used in the charge injection layer may be appropriately selected depending on a relation with the material of an electrode and an adjacent layer, and examples are polyaniline or its derivatives, polythiophene or its derivatives, polypyrrole and its derivatives, polyphenylenevinylene and its derivatives, polythienylenevinylene and its derivatives, polyquinoline and its derivatives, polyquinoxaline and its derivatives, electric conductive polymers such as polymers containing an aromatic amine structure on the side chain or main chain, metal phthalocyanine (copper phthalocyanine and the like), carbon and the like.

An insulation layer having a thickness of 2 nm or less has a function of making charge injection easier. The material of the above-described insulation layer includes a metal fluoride, metal oxide, organic insulating material and the like. Polymer LED carrying an insulation layer having a thickness of 2 nm or less provide thereon includes polymer LED in which an insulation layer having a thickness of 2 nm or less is placed next to a cathode, and polymer LED in which an insulation layer having a thickness of 2 nm or less is placed next to an anode.

Specifically, the following structures q) to ab) are mentioned, for example.

q) anodeAnode/insulation layer having a thickness of 2 nm or less/light emitting layer/cathode

r) anode/light emitting layer/insulation layer having a thickness of 2 nm or less/cathode

s) anode/insulation layer having a thickness of 2 nm or less/light emitting layer/insulation layer having a thickness of 2 nm or less/cathode

t) anode/insulation layer having a thickness of 2 nm or less/hole injection layer/light emitting layer/cathode

u) anode/hole injection layer/light emitting layer/insulation layer having a thickness of 2 nm or less/cathode

v) anode/insulation layer having a thickness of 2 nm or less/hole transporting layer/light emitting layer/insulation layer having a thickness of 2 nm or less/cathode

w) anode/insulation layer having a thickness of 2 nm or less/light emitting layer/electron transporting layer/cathode

x) anode/light emitting layer/electron transporting layer/insulation layer having a thickness of 2 nm or less/cathode

y) anode/insulation layer having a thickness of 2 nm or less/light emitting layer/electron transporting layer/insulation layer having a thickness of 2 nm or less/cathode

z) anode/insulation layer having a thickness of 2 nm or less/hole transporting layer/light emitting layer/electron transporting layer/cathode

aa) anode/hole transporting layer/light emitting layer/electron transporting layer/insulation layer having a thickness of 2 nm or less/cathode

ab) anode/insulation layer having a thickness of 2 nm or less/hole transporting layer/light emitting layer/electron transporting layer/insulation layer having a thickness of 2 nm or less/cathode

The polymer LED of the present invention includes those having a device structure as illustrated in the above-described a) to ab) in which a polymer compound of the present invention is contained in any of the hole injection layer, hole transporting layer, light emitting layer, electron transporting layer and electron injection layer.

The substrate which forms polymer LED of the present invention may advantageously be that forming an electrode and which does not change in forming a layer of an organic substance, and examples thereof include glass, plastic, polymer film, silicon substrate and the like. In the case of an opaque substrate, it is preferable that the opposite electrode is transparent or semi-transparent.

Usually, at least one of an anode and a cathode contained in polymer LED of the present invention is transparent or semi-transparent. It is preferable, that a cathode is transparent or semi-transparent.

The material of the cathode includes an electric conductive metal oxide film, semi-transparent metal thin film and the like. Films (NESA and the like) formed using electric conductive glass composed of indium oxide, zinc oxide, tin oxide, and composite thereof: indium.tin.oxide (ITO), indium.zinc.oxide and the like, gold, platinum, silver, copper and the like are used, and ITO, indium.zinc.oxide, tin oxide are preferable. The manufacturing method includes a vacuum vapor-deposition method, sputtering method, ion plating method, plating method and the like. The anode includes organic transparent electric conductive films made of polyaniline or its derivative, polythiophene or its derivative, and the like.

The thickness of an anode can be appropriately selected in view of light transmission and electric conductivity, and it is, for example, from 10 nm to 10 μm, preferably 20 nm to 1 μm, further preferably 50 nm to 500 nm.

For making charge injection easier, a layer made of a phthalocyanine derivative, electric conductive polymer, carbon and the like, or a layer having an average thickness of 2 nm or less made of a metal oxide, metal fluoride, organic insulation material and the like, may be provided on an anode.

The material of a cathode used in polymer LED of the present invention preferably includes materials of small work function. For example, metals such as lithium, sodium, potassium, rubidium, cesium, beryllium, magnesium, calcium, strontium, barium, aluminum, scandium, vanadium, zinc, yttrium, indium, cerium, samarium, europium, terbium, ytterbium and the like, alloys made of two or more of them, or alloys made of at least one of them and at least one of gold, silver, platinum, copper, manganese, titanium, cobalt, nickel, tungsten and tin, graphite or graphite intercalation compounds and the like are used. Examples of the alloy include magnesium-silver alloy, magnesium-indium alloy, magnesium-aluminum alloy, indium-silver alloy, lithium-aluminum alloy, lithium-magnesium alloy, lithium-indium alloy, calcium-aluminum alloy and the like. The cathode may take a laminated structure including two or more layers.

The thickness of a cathode can be appropriately selected in view of electric conductivity and durability, and it is, for example, from 10 nm to 10 μm, preferably 20 nm to 1 μm, further preferably 50 nm to 500 nm.

The cathode manufacturing method includes a vacuum vapor-deposition method, sputtering method, lamination method of thermally press-bonding a metal thin film, and the like. A layer made of an electric conductive polymer, or a layer having an average thickness of 2 nm or less made of a metal oxide, metal fluoride, organic insulation material and the like, may be provided between a cathode and an organic substance layer, and after manufacturing a cathode, a protective layer for protecting the polymer LED may be installed. For use of the polymer LED stably for a long period of time, it is preferable to install a protective layer and/or protective cover, for protecting a device from outside.

The protective layer includes a conjugated polymer compound, metal oxide, metal fluoride, metal boride and the like. The protective cover includes a glass plate, and a plastic plate having a surface subjected to low water permeation treatment, and the like, and a method of pasting the cover to a device substrate with a thermosetting resin or photo-curable resin to attain close sealing is suitably used. When a space is maintained using a spacer, prevention of blemishing of a device is easier. If an inert gas such as nitrogen, argon and the like is filled in this space, oxidation of a cathode can be prevented, further, by placing a drying agent such as barium oxide and the like in this space, it becomes easy to suppress moisture adsorbed in a production process from imparting damage to the device. It is preferable to adopt one strategy among these methods.

The polymer LED of the present invention can be used as a sheet light source, segment display, dot matrix display, and back light of a liquid crystal display.

For obtaining light emission in the form of sheet using polymer LED of the present invention, it may be advantages to place a sheet anode and a sheet cathode so as to overlap. For obtaining light emission in the form of pattern, there are a method in which a mask having a window in the form of pattern is placed on the surface of the above-described sheet light emitting device, a method in which an organic substance layer in non-light emitting parts is formed with extremely large thickness to give substantially no light emission, a method in which either anode or cathode, or both electrodes are formed in the form pattern. By forming a pattern by any of these methods, and placing several electrodes so that on/off is independently possible, a display of segment type is obtained which can display digits, letters, simple marks and the like. Further, for providing a dot matrix device, it may be permissible that both an anode and a cathode are formed in the form of stripe, and placed so as to cross. By using a method in which several polymer fluorescent bodies showing different emission colors are painted separately or a method in which a color filter or a fluorescence conversion filter is used, partial color display and multi-color display are made possible. In the case of a dot matrix device, passive driving is possible, and active driving may also be carried out in combination with TFT and the like. These displays can be used as a display of a computer, television, portable terminal, cellular telephone, car navigation, view finder of video camera, and the like.

Further, the above-described sheet light emitting device is of self emitting and thin type, and can be suitably used as a sheet light source for back light of a liquid crystal display, or as a sheet light source for illumination. If a flexible substrate is used, it can also be used as a curved light source or display.

Examples will be shown below for illustrating the present invention further in detail, but the invention is not limited to them.

### (Number-Average Molecular Weight and Weight-Average Molecular Weight)

Here, as the number-average molecular weight and the weight-average molecular weight, polystyrene-reduced number-average molecular weight and polystyrene-reduced weight-average molecular weight were measured by GPC (manufactured by Shimadzu Corp., LC-10 Avp). A polymer to be measured was dissolved in tetrahydrofuran so as to give a concentration of about 0.5 wt %, and the solution was injected in an amount of 50 μL into GPC. Tetrahydrofuran was used as the mobile phase of GPC, and allowed to flow at a flow rate of 0.6 mL/min. In the column, two TSKgel Super HM-H (manufactured by Tosoh Corp.) and one TSKgel Super H2000 (manufactured by Tosoh Corp.) were connected serially. A differential refractive index detector (RID-10A: manufactured by Shimadzu Corp.) was used as a detector.

### (Fluorescent Spectrum)

Fluorescent spectrum was measured according to the following method. A 0.8 wt % toluene or chloroform solution of a polymer was spin-coated on quartz to form a thin film of the polymer. This thin film was excited at a wavelength of 350 nm, and fluorescent spectrum was measured using a fluorescence spectrophotometer (Fluorolog manufactured by Horiba, Ltd.). For obtaining relative fluorescence intensity in the thin film, fluorescent spectrum plotted against wave number was integrated in the spectrum measuring range utilizing the intensity of Raman line of water as a standard, and measurement was performed using a spectrophotometer (Cary 5E, manufactured Varian), obtaining a value allocated to the absorbance at the excited wavelength.

### (Glass Transition Temperature)

The glass transition temperature was measured by DSC (DSC2920, manufactured by TA Instruments)

### (Measurement of LUMO)

For measurement of LUMO of a conjugated polymer compound, cyclic voltammetry (ALS600, manufactured by PAS) was used, and measurement was performed in an acetonitrile solvent containing 0.1 wt % tetrabutylammonium-tetrafluoroborate. A conjugated polymer compound was dissolved in chloroform to give a concentration of about 0.2 wt %, then, a chloroform solution of the conjugated polymer compound was applied in an amount of 1 mL on an action electrode, and chloroform was vaporized to form a thin film of the conjugated polymer compound. For measurement, a silver/silver ion electrode as a reference electrode, glassy carbon electrode as an action electrode, and a platinum electrode as a counter electrode, were used, and measurement was effected in a glove box purged with nitrogen. The sweeping rates of potential were both measured at 50 mV/s. LUMO was calculated from the reduction potential obtained by the cyclic voltammetry.

### Synthesis Example 1

### (Synthesis of Compound 1)

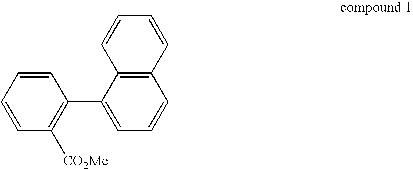

Into a 10 L separable flask purged with an argon gas was added 619 g of methyl bromobenzoate, 904 g of potassium carbonate and 450 g of 1-naphthylboronic acid, and 3600 ml of toluene and 4000 ml of water were added and the mixture was stirred. 30 g of tetrakistriphenylphosphinepalladium (0) was added and the mixture was refluxed under heat, and stirring the mixture continued for 3 hours. After cooling to room temperature, the mixture was separated, and washed with 2000 ml of water. The solvent was distilled off, then, purification by silica gel column was carried out using toluene. The resultant curd was concentrated and washed with 774 ml of hexane twice, and dried to obtain 596.9 g of compound 1 as while solid.

### (Synthesis of Compound 2)

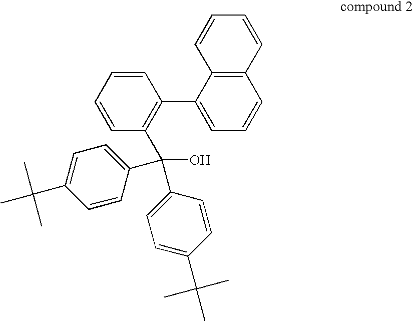

Into a 3 L three-necked flask was added 113 g of 4-t-butylphenyl bromide and 1500 ml of tetrahydrofuran, and the mixture was cooled down to −78° C. under a nitrogen atmosphere. 600 ml of n-butyllithium was charged into a dropping funnel and n-butyllithium was dropped slowly so as not to cause change of temperature in the system. After dropping, the mixture was stirred for 2 hours at room temperature, then, cooled down to −78° C., and a solution prepared by dissolving 34.6 g of the compound 1 in 500 ml of tetrahydrofuran was dropped over a period of 60 minutes. Further, the mixture was stirred for 2 hours at −78° C., then, the reaction was stopped using 500 ml of a saturated ammonium chloride aqueous solution, and extracted with 1000 ml of toluene. After washing with water, impurities were removed by passing through a silica gel short column, to obtain 61.5 g of compound 2.

### (Synthesis of Compound 3)

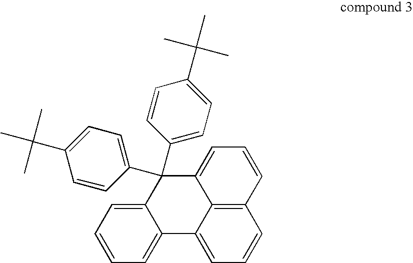

Into a 2000 ml three-necked flask charged with 325 mL of a boron trifluoride ether complex was added 1500 ml of dichloromethane, and the mixture was cooled sufficiently with an ice bath. 132 g of the compound 2 was used to prepare a dichlomethane solution which was dropped over a period of 1 hour using a non-isobaric dropping funnel. The ice bath was removed, and the mixture was stirred for 2 hours at room temperature, then, water was added to this to stop the reaction. Extraction was performed using chloroform, and the organic layer was concentrated, then, orange oil was obtained. Re-crystallization was performed using 240 ml of toluene and 50 ml of 2-propanol, to obtain 36.2 g of the intended compound 3.

### Example 1

### (Synthesis of Compound 4)

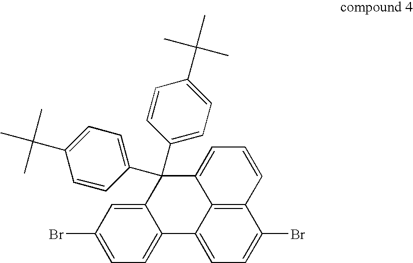

Under a nitrogen atmosphere, 20 g (purity: 99.6%) of the ring-closed body 3 was charged in a 1000 ml three-necked flask, and 100 ml of dichloromethane was added to dissolve this, and 259 ml of acetic acid was added and the mixture was heated at 50° C. in an oil bath. While heating, 11.2 g of zinc chloride was added and the mixture was stirred, and a solution prepared by dissolving 35.4 g of benzyltrimethylammonium tribromide in 150 ml of dichloromethane was added over 60 minutes while refluxing under heat. Further, the mixture was stirred for 1 hour at 50° C., and cooled down to room temperature, then, 200 ml of water was added to stop the reaction. The liquid was separated, and the aqueous layer was extracted with 300 ml of chloroform, and the organic layers were combined. The organic layer was washed with 300 ml of a saturated sodium thiosulfate aqueous solution, then, washed with 500 ml of a saturated sodium hydrogen carbonate aqueous solution and 200 ml of water. The resultant organic layer was filtrated by passing through pre-coated silica gel. The solvent was distilled off, and the resultant mixture was re-crystallized from hexane, to obtain 17.2 g of an intended compound 4 as while solid.

### <Analysis>

¹H-NMR (300 MHz/CDCl₃): d 1.27 (s, 18H), 6.79(dd, 2H), 7.15-7.23 (m, 3H), 7.48 (ddd, 1H), 7.54 (td, 1H), 7.81 (dd, 1H), 7.86 (d, 1H), 7.88 (d, 1H), 8.16 (dt, 1H).

LC/MS (APPI(pos)): m/z calcd for [C_{3 7}H_{3 4}Br₂]^{+·}, 638.47; found, 638.0.

### Synthesis Example 2

### Synthesis of 1-bromo-4-t-butyl-2,6-dimethylbenzene

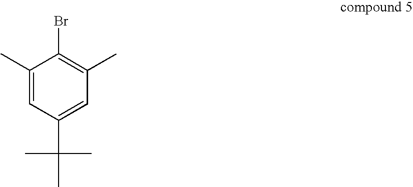

Under an inert atmosphere, into a 500 ml of three-necked flask was charged 225 g of acetic acid, and 24.3 g of 5-t-butyl-m-xylene was added. Subsequently, 31.2 g of bromine was added, then, reacted at 15 to 20° C. for 3 hours.

The reaction liquid was added into 500 ml of water and the deposited precipitate was filtrated. The product was washed with 250 ml of water twice, to obtain 34.2 g of compound 5 as white solid.

### (Synthesis of Compound 6)

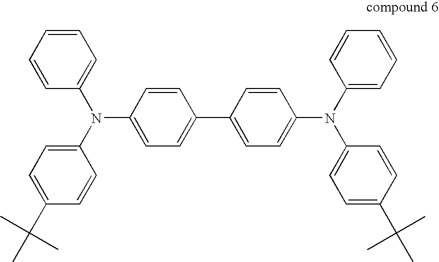

Under an inert atmosphere, into a 300 ml of three-necked flask was charged 1660 ml of deaerated dehydrated toluene, and 275.0 g of N,N′-diphenylbenzidine and 449.0 g of 4-t-butyl-2,6-dimethylbromobenzene. Subsequently, 7.48 g of tris(dibenzylideneacetone)dipalladium and 196.4 g of t-butoxysodium, then, 5.0 g of tri(t-butyl)phosphine was added. Thereafter, the mixture was reacted at 105° C. for 7 hours.

To the reaction liquid was added 2000 ml of toluene, and filtration was performed through cerite, the filtrate was washed with 1000 ml of water three times, then, concentrated to 700 ml. To this was added 1600 ml of a toluene/methanol (1:1) solution, and the deposited crystal was filtrated, and washed with methanol. 479.4 g of while solid was obtained.

### (Synthesis of Compound 7)

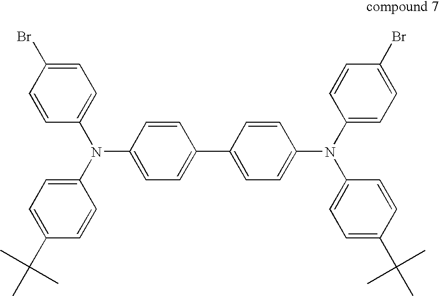

Under an inert atmosphere, 472.8 g of the above-described N,N′-diphenyl-N,N′-bis(4-t-butyl-2,6-dimethylphenyl)-benzidine was dissolved in 4730 g of chloroform, then, 281.8 g N-bromosuccinimide was charged in 12-divided portions over a period of 1 hour under shading and ice bath, and reacted for 3 hours.

1439 ml of chloroform was added to the reaction liquid, and the mixture was filtrated, and the filtrate chloroform solution was washed with 2159 ml of 5% sodium thiosulfate, and toluene was distilled off to obtain a white crystal. The resultant white crystal was re-crystallized from toluene/ethanol, to obtain 678.7 g of a white crystal. MS (APCI(+)): (M+H)⁺ 815.2

### Example 2

### Synthesis of Polymer Compound 1

The compound 4 (1.92 g) and 2,2′-bipyridyl (1.27 g) were dissolved in 216 mL of dehydrated tetrahydrofuran, then, an atmosphere in the system was purged with nitrogen by bubbling with nitrogen. Under a nitrogen atmosphere, this solution was heated up to 60° C., and bis(1,5-cyclooctadiene)nickel(0) {Ni(COD)₂} (2.23g) was added, and reacted for 3 hours at 60° C. while stirring. This reaction liquid was cooled down to room temperature (about 25° C.), and dropped into a 25% ammonia water 11 mL/methanol 216 mL/ion exchanged water 216 mL mixed solution, and the mixture was stirred for 1 hour, then, the deposited precipitate was filtrated and dried under reduced pressure for 2 hours, then, dissolved in about 100 mL of toluene before performing filtration, and the filtrate was purified by passing through an alumina column, and about 200 ml of 5.2% hydrochloric acid water was added, and the mixture was stirred for 3 hours, then, the aqueous layer was removed. Next, about 200 mL of 4% ammonia water was added, and the mixture was stirred for 2 hours, then, the aqueous layer was removed. Further, to the organic layer was added about 200 mL of ion exchanged water and the mixture was stirred for 1 hour, then, the aqueous layer was removed. The organic layer was dropped into about 600 mL of methanol and the mixture was stirred for 1 hour, and the deposited precipitate was filtrated and dried under reduced pressure for 2 hours. The yield of the resultant copolymer (hereinafter, referred to as polymer compound 1) was 0.080 g. The polystyrene-reduced number average molecular weight and the polystyrene-reduced weight average molecular weight were Mn=6.5×10⁴ and Mw=3.0×10⁵, respectively.

### Example 3

### Synthesis of Polymer Compound 2

The compound 4 (0.82 g), N,N′-bis(4-bromophenyl)-N,N′-bis(4-t-butyl-2,6-dimethylphenyl)-benzidine (0.49 g) (compound 7) and 2,2′-bipyridyl (0.84 g) were dissolved in 144 mL of dehydrated tetrahydrofuran, then, an atmosphere in the system was purged with nitrogen by bubbling with nitrogen. Under a nitrogen atmosphere, this solution was heated up to 60° C., and bis(1,5-cyclooctadiene)nickel(0) {Ni(COD)₂} (1.49 g) was added, and reacted for 3 hours at 60° C. while stirring. This reaction liquid was cooled down to room temperature (about 25° C.), and dropped into a 25% ammonia water 7 mL/methanol 144 mL/ion exchanged water 144 mL mixed solution, and the mixture was stirred for 1 hour, then, the deposited precipitate was filtrated and dried under reduced pressure for 2 hours, then, dissolved in about 60 mL of toluene before performing filtration, and the filtrate was purified by passing through an alumina column, and about 120 ml of 5.2% hydrochloric acid water was added, and the mixture was stirred for 3 hours, then, the aqueous layer was removed. Next, about 120 mL of 4% ammonia water was added, and the mixture was stirred for 2 hours, then, the aqueous layer was removed. Further, to the organic layer was added about 120 mL of ion exchanged water and the mixture was stirred for 1 hour, then, the aqueous layer was removed. The organic layer was dropped into about 400 mL of methanol and the mixture was stirred for 1 hour, and the deposited precipitate was filtrated and dried under reduced pressure for 2 hours. The yield of the resultant copolymer (hereinafter, referred to as polymer compound 2) was 0.420 g. The polystyrene-reduced number average molecular weight and the polystyrene-reduced weight average molecular weight were Mn=2.0×10⁴ and Mw=1.5×10⁵, respectively.

### Synthesis Example 4

### Synthesis of Polymer Compound 3

Under an inert atmosphere, 2,7-dibromo-9,9-dioctylfluorene (287 mg, 0.523 mmol), cyclic ethylene glycol 2,7-(9,9-dioctyl)fluorenediboronate (305 mg, 0.575 mmol) and Aliquot 336 (15 mg) were dissolved in toluene (4.3 g), and to this was added about 1 g of an aqueous solution of potassium carbonate (231 mg, 1.67 mmol). Further, tetrakis(triphenylphosphine)palladium (0.39 mg, 0.00034 mmol) was added and the mixture was refluxed under heat for 20 hours. Subsequently, bromobenzene (11.5 mg) was added, and the mixture was further refluxed under heat for 5 hours. After completion of heating, the reaction mass was dropped into mixed liquid of methanol (40 ml) and 1 N hydrochloric acid water (2.2 ml), and the deposited precipitate was filtrated off. The resultant precipitate was washed with methanol and water, and subjected to drying under reduced pressure, to obtain solid. Subsequently, the solid was dissolved in 50 ml of toluene, and the resultant liquid was passed through a silica column, then, concentrated to 20 ml. The concentrated liquid was dropped into methanol, the deposited precipitate was filtrated off, and dried under reduced pressure to obtain polymer compound 3. Yield: 340 mg.

The resultant polymer compound 3 had polystyrene-reduced molecular weights: Mn=1.2×10³, Mw=3.2×10³.

### Example 4

### Measurement of Fluorescent Spectrum

The fluorescent spectrum of the polymer compound 1 was measured by the above-described method, to obtain fluorescent spectrum showing a peak at 470 nm.

### Example 5

### Measurement of Fluorescent Spectrum

The fluorescent spectrum of the polymer compound 2 was measured by the above-described method, to obtain fluorescent spectrum showing a peak at 478 nm.

### Example 6

### Eelectron Injection Property Evaluation

The LUMO value of the polymer compound 1 was measured under the above-described conditions, to find a value of 2.93 eV.

### Example 7

### Electron Injection Property Evaluation

The LUMO value of the polymer compound 2 was measured under the above-described conditions, to find a value of 2.93 eV.

### Comparative Example 1

The LUMO value of the polymer compound 3 was measured under the above-described conditions, to find a value of 2.44 eV.

It is understood that the polymer compounds 1, 2, 4 and 5 together show excellent electron injection property.

TABLE 1

| | | Weight | |
|---|---|---|---|
| | | average | |
| | | molecular | |
| | | weight | LUMO value |
| Polymer | Example 3 | 300000 | 2.93 eV |
| compound 1 | | | |
| Polymer | Example 4 | 150000 | 2.92 eV |
| compound 2 | | | |
| Polymer | Example 9 | 719000 | 2.83 eV |
| compound 4 | | | |
| Polymer | Example 10 | 82000 | 2.75 eV |
| compound 5 | | | |
| Polymer | Comparative | 54000 | 2.44 eV |
| compound 3 | Example 1 | | |

### Synthesis Example 5

### (Synthesis of Compound 7)

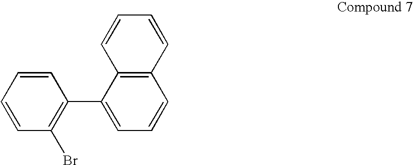

Into a three-necked round-bottomed flask (500 ml) was added 25.1 g of 2-bromoiodobenzene, 20.0 g of naphthaleneboronic acid, 0.427 g of tetrakistriphenylphosphinepalladium (0) and 25.5 g of potassium carbonate, then, 92 ml of toluene and 91 ml of water were added and the mixture was refluxed under heat. After stirring for 24 hours, the mixture was cooled down to room temperature. The reaction solution was filtrated by passing through silica gel, and the solvent was distilled off to obtain 25 g of a coarse product. It was purified by silica gel column chromatography, then, re-crystallized using hexane, to obtain 12.2 g of compound 7 as white solid.

### (Synthesis of Compound 8)

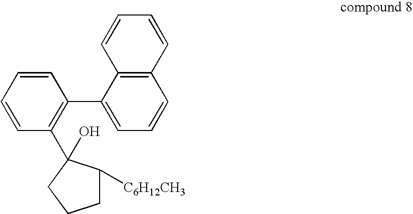

Under a nitrogen atmosphere, the compound 7 was charged into a three-necked flask (200 ml), and 73 ml of tetrahydrofuran was added to dissolve the compound. After cooling down to −78° C., 18.14 ml of n-butyllithium was added. After stirring for 30 minutes, propylcylohexanone was dissolved in 6.38 mL of THF and added to the stirred liquid. After raising the temperature up to room temperature, 50 ml of a saturated ammonium chloride aqueous solution was added to stop the reaction, and extraction with 100 ml of THF was performed. The resultant organic layer was passed through pre-coated silica gel to concentrate. It was purified by silica gel column chromatography, to obtain 7.5 g of compound 8.

### <Analysis>

¹H-NMR (300 MHz/CDCl₃) d 0.86-0.94 (m, 3H), 1.28-1.90 (m, 17H), 1.95-2.10 (m, 1H), 2.15-2.35 (m, 1H), 7.10 (d, 1H), 7.26-7.51 (m, 7H), 7.61 (dd, 1H), 7.86 (t, 1H), 7.86 (t, 1H).

LC-MS (APPI-posi): m/z calcd. for [C28H34O], 386.57, found for [C28H34O]^{+·}, 387.2

### (Synthesis of Compound 9)

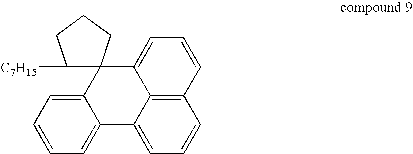

Under a nitrogen atmosphere, 12.5 g of BF3Et2O and 40 ml of dichloromethane were charged into a 100 ml two-necked flask which was cooled in an ice bath. The compound 8 was dissolved in 15 ml of dichloromethane and dropped into the liquid, and the resultant mixture was stirred for 30 minutes. 50 mL of water was added to stop the reaction, and extraction with 50 mL of dichloromethane was performed twice. The resultant solution was concentrated, and purified by silica gel column chromatography, to obtain 2 g of compound 9.

### <Analysis>

¹H-NMR (300 MHz/CDCl₃) d 0.80 (t, 3H), 1.09-1.26 (m, 10H), 1.31-1.45 (m, 4H), 1.63-1.78 (m, 1H), 1.84 (q, 1H), 2.05 (m, 1H), 2.54 (m, 1H), 2.84 (t, 1H), 7.23-7.35 (m, 3H), 7.42-7.53 (m, 2H), 7.56 (d, 1H), 7.77 (d, 1H), 7.85 (t, 1H), 7.91 (d, 1H), 8.54-8.57 (m, 1H).

### Example 8

### (Synthesis of Compound 10)

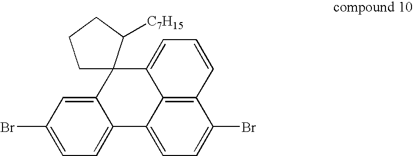

Under a nitrogen atmosphere, the compound 9 was charged into a 100 ml three-necked flask, and 33 ml of dichloromethane and 33 ml of acetic acid were added and the mixture was heated at 50° C. Under heating, zinc chloride was charged, and 4.45 g of benzyltrimethylammonium tribromide was dissolved in 33 ml of dichloromethane to prepare a solution which was dropped into the mixture while refluxing under heat. After stirring for 30 minutes, the mixture was allowed to cool to room temperature, and water was added to stop the reaction, and extraction was performed with chloroform. The resultant organic layer was washed with 50 ml of a saturated sodium thiosulfate aqueous solution twice, further, washed with 100 ml of a saturated sodium hydrogen carbonate aqueous solution and 50 ml of water. The organic solvent was concentrated. After purifying by silica gel column chromatography, re-crystallization was carried out, to obtain 1 g of compound 10.

### <Analysis>

¹H-NMR (300 MHz/CDCl₃) d 0.83 (t, 3H), 1.0-1.24 (m, 11H), 1.3-1.53 (m, 3H), 1.6-1.77 (m, 1H), 1.8-1.94 (m, 1H), 2.0-2.12 (m, 1H), 2.4-2.54 (dd, 1H), 2.56-2.85 (t, 1H), 7.43-7.47 (m, 1H), 7.50 (s, 1H), 7.50-7.61 (m, 2H), 7.70 (d, 1H), 7.83 (s, 1H), 8.29 (d, 1H), 8.43 (d, 1H). under re-measurement

LC-MS (APPI-posi): m/z calcd for [C28H30Br2], 526.35; found for [C28H30Br2]+·, 524.

### Example 9

### Synthesis of Polymer Compound 4

Into a reaction vessel (200 mL), 0.316 g of the compound 10 and 2,2′-bipyridyl (0.159 g) were charged under a nitrogen atmosphere, then, tetrahydrofuran (43 mL) was added to prepare a solution, and 0.281 g of bis(1,5-cyclooctadiene)nickel(0) {Ni(COD)₂} was charged. The temperature was raised up to 60° C. while stirring, and the mixture was stirred for 3 hours. This reaction liquid was cooled down to room temperature (about 25° C.), and dropped into a 25% ammonia water 2 mL/methanol 43 mL/ion exchanged water 43 mL mixed solution, and the mixture was stirred for 1 hour, then, the deposited precipitate was filtrated and dried under reduced pressure for 2 hours, then, dissolved in about 20 mL of toluene before performing filtration using a glass filter pre-coated with radiolite, the filtrate was purified by passing through an alumina column, about 35 ml of 5.2% hydrochloric acid water was added, and the mixture was stirred for 3 hours, then, the aqueous layer was removed. Next, about 35 mL of 4% ammonia water was added, and the mixture was stirred for 2 hours, then, the aqueous layer was removed. Further, to the organic layer was added about 35 mL of ion exchanged water and the mixture was stirred for 1 hour, then, the aqueous layer was removed. The organic layer was dropped into about 120 mL of methanol and the mixture was stirred for 1 hour, and the deposited precipitate was filtrated and dried under reduced pressure for 2 hours. The yield of the resultant polymer compound 4 was 0.140 g. The polystyrene-reduced number average molecular weight, the polystyrene-reduced weight average molecular weight and the z-average molecular weight were Mn=1.56×10⁵, Mw=7.19×10⁵ and Mz=1.66×10⁶, respectively.

### Example 10

### Synthesis of Polymer Compound 5

Into a reaction vessel (200 mL), 0.184 g of the compound 10, N,N′-bis(4-bromophenyl)-N,N′-bis(4-t-butyl-2,6-dimethylphenyl)-benzidine (0.49 g)(compound 7)(0.111g) and 2,2′-bipyridyl (0.133 g) were charged, then, dehydrated tetrahydrofuran (54 mL) previously bubbled (10 minutes) with an argon gas under a nitrogen atmosphere was added to prepare a solution, and bis(1,5-cyclooctadiene)nickel(0) {Ni(COD)₂} (0.234 g) was charged. The temperature was raised up to 60° C. while stirring, and the mixture was stirred for 3 hours. This reaction liquid was cooled down to room temperature (about 25° C.), and dropped into a 25% ammonia water 2 mL/methanol 54 mL/ion exchanged water 54 mL mixed solution, and the mixture was stirred for 1 hour, then, the deposited precipitate was filtrated and dried under reduced pressure for 2 hours, then, dissolved in about 20 mL of toluene before performing filtration using a glass filter pre-coated with radiolite, the filtrate was purified by passing through an alumina column, about 30 ml of 5.2% hydrochloric acid water was added, and the mixture was stirred for 3 hours, then, the aqueous layer was removed. Next, about 30 mL of 4% ammonia water was added, and the mixture was stirred for 2 hours, then, the aqueous layer was removed. Further, to the organic layer was added about 30 mL of ion exchanged water and the mixture was stirred for 1 hour, then, the aqueous layer was removed. The organic layer was dropped into about 50 mL of methanol and the mixture was stirred for 1 hour, and the deposited precipitate was filtrated and dried under reduced pressure. The yield of the resultant polymer compound 5 was 0.150 g (yield: 70%). The polystyrene-reduced number average molecular weight, the polystyrene-reduced weight average molecular weight and the z-average molecular weight were Mn=2.1×10⁴, Mw=8.2×10⁴ and Mz=1.7×10⁵, respectively.

### Synthesis Example 6

### Synthesis of Polymer Compound 6

Into a reaction vessel (200 mL) of a small scale 6-consecutive reaction apparatus, the compound 10 (53 mg), 2,7-dibromo-9,9-dioctylfluorene (332 mg) and 2,2′-bipyridyl (266 mg) were charged, then, dehydrated tetrahydrofuran (47 mL) previously bubbled (10 minutes) with an argon gas under a nitrogen atmosphere was added to prepare a solution, and bis(1,5-cyclooctadiene)nickel(0) {Ni(COD)₂} (468 mg) was charged. The temperature was raised up to 60° C. while stirring, and the mixture was stirred for 3 hours. This reaction liquid was cooled down to room temperature (about 25° C.), and dropped into a 25% ammonia water 4 mL/methanol 72 mL/ion exchanged water 72 mL mixed solution, and the mixture was stirred for 1 hour, then, the deposited precipitate was filtrated and dried under reduced pressure for 2 hours, then, dissolved in about 30 mL of toluene, then, 120 mg of radiolite was added and the mixture was stirred, and filtration was performed using a Kiriyama funnel pre-coated with radiolite (2 mm), the filtrate was purified by passing through an alumina column, about 59 ml of 5.2% hydrochloric acid water was added, and the mixture was stirred for 3 hours, then, the aqueous layer was removed. Next, about 59 mL of 4% ammonia water was added, and the mixture was stirred for 2 hours, then, the aqueous layer was removed. Further, to the organic layer was added about 59 mL of ion exchanged water and the mixture was stirred for 1 hour, then, the aqueous layer was removed. The organic layer was dropped into about 94 mL of methanol and the mixture was stirred for 1 hour, and the deposited precipitate was filtrated and dried under reduced pressure for 2 hours. The yield of the resultant polymer compound 6 was 0.186 g. The polystyrene-reduced number average molecular weight, the polystyrene-reduced weight average molecular weight and the z-average molecular weight were Mn=1.28×10⁵, Mw=3.15×10⁵ and Mz=5.27×10⁵, respectively.

### Example 11

### Measurement of Fluorescent Spectrum

The fluorescent spectrum of the polymer compound 4 was measured by the above-described method, to find a fluorescent spectrum showing a peak at 454 nm.

### Example 12

### Measurement of Fluorescent Spectrum

The fluorescent spectrum of the polymer compound 5 was measured by the above-described method, to find a fluorescent spectrum showing a peak at 478 nm.

### Example 13

### Electron Injection Property Evaluation

The LUMO value of the polymer compound 4 was measured under the above-described conditions, to find a value of 2.83 eV.

### Example 14

### Electron Injection Property Evaluation

The LUMO value of the polymer compound 5 was measured under the above-described conditions, to find a value of 2.75 eV.

### Example 15

### Heat Resistance Evaluation

The glass transition point of the polymer compound 4 was measured under the above-described conditions, to find a temperature of 267° C.

### Example 16

### Heat Resistance Evaluation

The glass transition point of the polymer compound 5 was measured under the above-described conditions, to find a temperature of 295° C.

### Comparative Example 2

### Heat Resistance Evaluation

The glass transition point of the polymer compound 3 was measured under the above-described conditions, to find a temperature of 73° C.

TABLE 2

| | | Tg value |
|---|---|---|
| Polymer compound 4 | Example 15 | 267° C. |
| Polymer compound 5 | Example 16 | 295° C. |
| Polymer compound 3 | Comparative Example 2 | 72° C. |

It is understood that the polymer compounds 4 and 5 together show excellent heat resistance.

### INDUSTRIAL APPLICABILITY

The conjugated polymer compound of the present invention is useful as a light emitting material and a charge transporting material, and excellent in electron injection property. Therefore, polymer LED containing a conjugated polymer compound of the present invention can be used for back light of liquid crystal displays or curved or flat light sources for illumination, segment type displays, dot matrix flat panel displays and the like.

1. A conjugated polymer compound comprising as a partial structure therein a structure of the following formula (a): 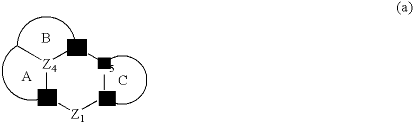
wherein, a ring A and a ring B represent each independently an aromatic ring optionally having a substituent or a non-aromatic ring optionally having a substituent, and at least one of the ring A and the ring B is an aromatic ring, a ring C represents an aromatic ring optionally having a substituent, Z₁ represents an atom selected from a carbon atom, oxygen atom, sulfur atom, nitrogen atom, silicon atom, boron atom, phosphorus atom and selenium atom or a group containing the atom, and Z₂ to Z₆ represent each independently an atom selected from a carbon atom, silicon atom, nitrogen atom and boron atom or a group containing the atom, and when the ring B and the ring C have a substituent, these substituents may be connected to form a ring.

2. The conjugated polymer compound according to claim 1 comprising as a partial structure a structure of the following formula (1) wherein Z₄ to Z₆ represent a carbon atom in the formula (a):

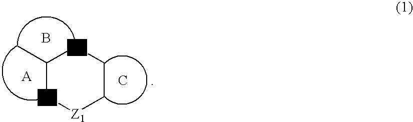

3. The conjugated polymer compound according to claim 1 comprising as a repeating unit a structure of the following formula (2): 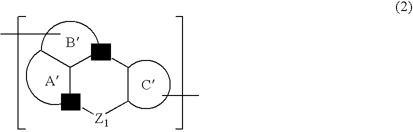
wherein, a ring B′ and a ring C′ represent each independently an aromatic ring optionally having a substituent, and a ring A′ represents an aromatic ring optionally having a substituent or a non-aromatic ring optionally having a substituent, two bonds are present on the ring B′ and the ring C′, and Z₁ to Z₃ represent the same meanings as described above.

4. The conjugated polymer compound according to claim 1 comprising as a repeating unit a structure of the following formula (3): 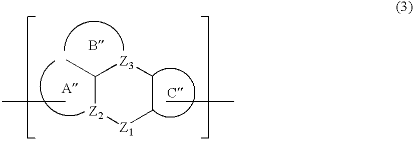
wherein, a ring A″ and a ring C″—represent each independently an aromatic ring optionally having a substituent, and a ring B″ represents an aromatic ring optionally having a substituent or a non-aromatic ring optionally having a substituent, two bonds are present on the ring A″ and the ring C″, and Z₁ to Z₃ represent the same meanings as described above.

5. The conjugated polymer compound according to claim 1 comprising a structure of the following formula (4): 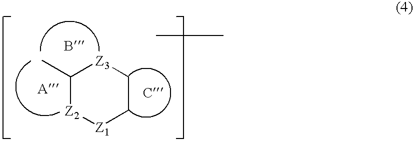
wherein, a ring A′″ and a ring B′″ represent an aromatic ring optionally having a substituent or a non-aromatic ring optionally having a substituent, and at least one of the ring A′″ and the ring B′″ is an aromatic ring optionally having a substituent, a ring C′″ represents an aromatic ring optionally having a substituent, a bond is present on the ring A′″, the ring B′″ or the ring C′″, and Z₁ to Z₃ represent the same meanings as described above.

6. The conjugated polymer compound according to claim 1 comprising a structure of the following formula (5): 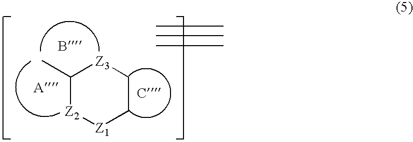
(wherein, a ring A″″ and a ring B″″ represent each independently an aromatic ring optionally having a substituent or a non-aromatic ring optionally having a substituent, and at least one of the ring A and the ring B is an aromatic ring optionally having a substituent, a ring C″″ represents an aromatic hydrocarbon ring optionally having a substituent, and three bonds are present on the ring A″″, the ring B″″ or the ring C″″, and Z₁ to Z₃ represent the same meanings as described above.

7. The conjugated polymer compound according to claim 1, wherein Z₂ and Z₃ are each independently >CH—, >CR′—, >C═, >SiH—, >SiR′—, >N—, ═N— or >B—, wherein R's represent each independently an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, nitro group, amide group, acid imide group, monovalent heterocyclic group, carboxyl group, substituted carboxyl group or cyano group.

8. The conjugated polymer compound according to claim 1, wherein Z₁ is —C(R_{w})(Rₓ)—, >C═C(R_{w})(Rₓ), —O—, —S—, —S(═O)—, —S(═O)(═O)—, —N(R_{w})—, —Si(R_{w})(Rₓ)—, —P(═O)(R_{w})—, —P(R_{w})—, —B(R_{w})—, —C(R_{w})(Rₓ)—O—, —C(═O)—O—, —C(R_{w})═N— or —Se— (wherein, R_{w} and Rₓ represent each independently a substituent.

9. The conjugated polymer compound according to claim 1, wherein all of Z₁ to Z₃ represent a carbon atom.

10. The conjugated polymer compound according to claim 1, wherein elements constituting all of the ring A, the ring B and the ring C represent a carbon atom.

11. The conjugated polymer compound according to claim 1, wherein the ring A, the ring B and the ring C represent each independently a benzene ring or naphthalene ring.

12. The conjugated polymer compound according to claim 11, wherein all of the ring A, the ring B and the ring C represent a benzene ring.

13. The conjugated polymer compound according to claim 1, comprising a repeating unit of the following formula (6) or (7): 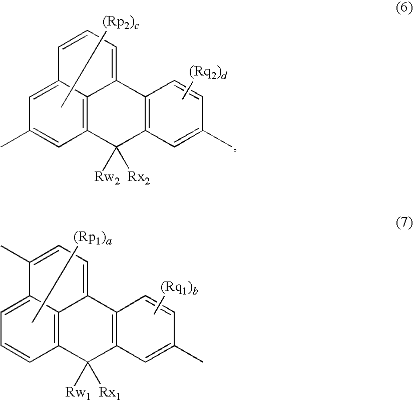
wherein, Rₚ₁, R_{q1}, Rₚ₂, R_{q2}, R_{w1}, Rₓ₁, R_{w2} and Rₓ₂ represent each independently a substituent, a and c represent each independently an integer of 0 to 5, and b and d represent each independently an integer of 0 to 3, and Rₚ₁ and R_{q1}, Rₚ₂ and R_{q2}, R_{w1} and Rₓ₁, and R_{w2} and Rₓ₂ each may be mutually connected to form a ring.

14. The conjugated polymer compound according to claim 13, wherein the carbon number of at least one of R_{w1} and Rₓ₁, and the carbon number of at least one of R_{w2} and Rₓ₂ are 2 or more.

15. The polymer compound according to claim 1, comprising at least one repeating unit other than said formulae (2) and (3).

16. The conjugated polymer compound according to claim 15, wherein the repeating unit other than said formulae (2) and (3) is selected from repeating units of the following formulae (8) to (11):
—Ar₁— (8)
—(Ar₂—X₁)ₑ—Ar₃— (9)
—Ar₄—X₂— (10)
—X₃— (11)
wherein Ar₁, Ar₂, Ar₃ and Ar₄ represent each independently an arylene group, divalent heterocyclic group or divalent group having a metal complex structure, X₁, X₂ and X₃ represent each independently —CR₁═CR₂—, —N(R₃)— or —(SiR₄ R₅)ₙ, R₂ and R₂ represent each independently a hydrogen atom, alkyl group, aryl group, monovalent heterocyclic group, carboxyl group, substituted carboxyl group or cyano group, R₃, R₄ and R₅ represent each independently a hydrogen atom, alkyl group, aryl group, monovalent heterocyclic group, arylalkyl group or substituted amino group, e represents an integer of 0 to 2, n represents an integer of 1 to 12, and when there are two or more R₁s, R₂s, R₃s, R₄s or R₅s, respectively, they may be the same or different.

17. The conjugated polymer compound according to claim 16, wherein the repeating unit of said formula (8) is a repeating unit of the following formula (12): 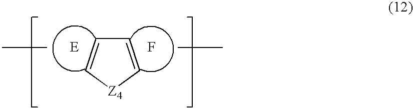
wherein a ring E and a ring F represent an aromatic ring, two -bonds are present on the ring E or the ring F, and Z₄ represents —C(R_{w})(Rₓ)—, >C═C(R_{w})(Rₓ), —O—, —S—, —S(═O), —S(═O)(═O)—, —N(R_{w})—, —Si(R_{w})(Rₓ)—, —P(═O)(R_{w})—, —P(R_{w})—, —B(R_{w})—, —C(R_{w})(Rₓ)—O—, —C(═O)—O—, —C(R_{w})═N— or —Se—. R_{w} and Rₓ represent each independently a substituent.

18. The conjugated polymer compound according to claim 16, wherein the repeating unit of said formula (8) is a repeating unit of any one of the following formulae (13) to (20): 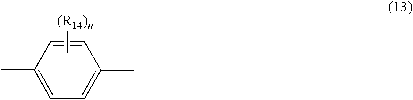
wherein, R₁₄ represents an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group or cyano group, n represents an integer of 0 to 4, and when there are two or more R₁₄s, they may be the same or different 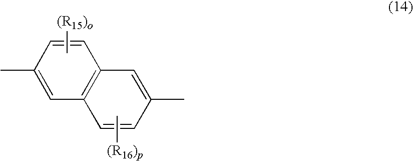
wherein R_{1 5} and R_{1 6} represent each independently an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group or cyano group, o and p represent each independently an integer of 0 to 3, and when there are two or more R₁₅s or R₁₆s, respectively, they may be the same or different, 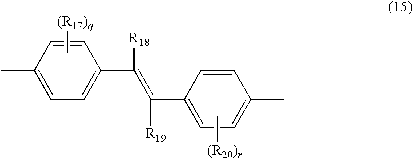
wherein R_{1 7} and R_{2 0} represent each independently an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group or cyano group, q and r represent each independently an integer of 0 to 4, R₁₈ and R₁₉ represent each independently a hydrogen atom, alkyl group, aryl group, monovalent heterocyclic group, carboxyl group, substituted carboxyl group or cyano group, and when there are two or more R₁₇s or R₂₀s, they may be the same or different, 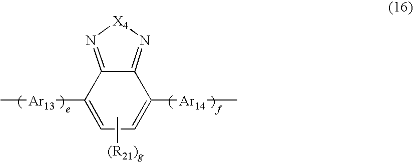
wherein R_{2 1} represents an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group or cyano group, g represents an integer of 0 to 2, Ar₁₃ and Ar₁₄ represent each independently an arylene group, divalent heterocyclic group or divalent group having a metal complex structure, e and f represent each independently 0 or 1, X₄ represents O, S, SO, SO₂, Se or Te, and when there are two or more R₂₁s, they may be the same or different, 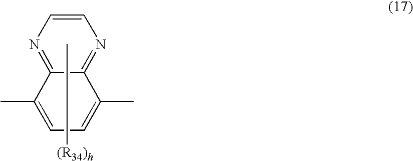
wherein R_{3 4} represents an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group or cyano group, h represents an integer of 0 to 4, and when there are two or more R₃₄s, they may be the same or different, 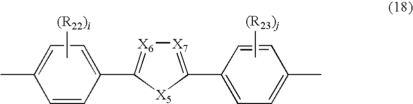
wherein R_{2 2} and R_{2 3} represent each independently an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group or cyano group, i and j represent each independently an integer of 0 to 4, X₅ represents O, S, SO₂, Se, Te, N—R₂₄, or SiR₂₅R₂₆, X₆ and X₇ represent each independently N or C—R₂₇. R₂₄, R₂₅, R₂₆ and R₂₇ represent each independently a hydrogen atom, alkyl group, aryl group, arylalkyl group or monovalent heterocyclic group, and when there are two or more R₂₅s, R₂₆s or R₂₇s, they may be the same or different, 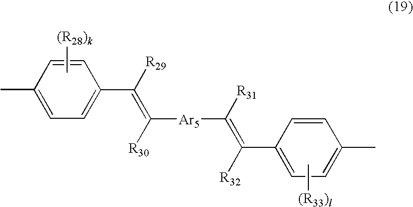
wherein R_{2 8} and R_{3 3} represent each independently an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group or cyano group, k and l represent each independently an integer of 0 to 4, R₂₉, R₃₀, R₃₁ and R₃₂ represent each independently a hydrogen atom, alkyl group, aryl group, monovalent heterocyclic group, carboxyl group, substituted carboxyl group or cyano group, Ar⁵ represents an arylene group, divalent heterocyclic group or divalent group having a metal complex structure, and when there are two or more R₂₈s or R₃₃s, they may be the same or different.

19. The conjugated polymer compound according to claim 16, wherein the repeating unit of said formula (9) is a repeating unit of the following formula (20): 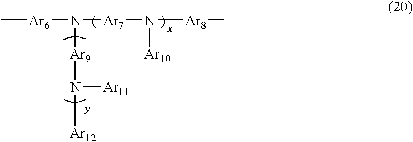
wherein Ar₆, Ar₇, Ar₈ and Ar₉ represent each independently an arylene group or divalent heterocyclic group, Ar₁₀, Ar₁₁ and Ar₁₂ represent each independently an aryl group or monovalent heterocyclic group, Ar₆, Ar₇, Ar₈, Ar₉ and Ar₁₀ may have a substituent, x and y represent each independently 0 or 1, and 0≦x+y≦1.

20. The conjugated polymer compound according to claim 1, comprising as a repeating unit a structure of said formula (2) or (3) and a structure of said formula (20).

21. The conjugated polymer compound according to claim 1, wherein the polystyrene-reduced number average molecular weight is 10³ to 10⁸.

22. A compound of the following formula (27): 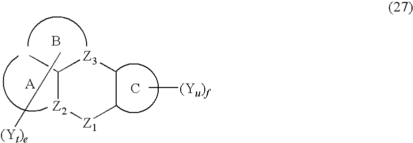
wherein a ring A, a ring B, a ring C and Z₁ to Z₃ are as described above, Yₜ and Yᵤ represent each independently a substituent, e and f represent each independently an inter of 0 or more, and e+f≧1 and e≦2, f≦1.

23. The compound according to claim 22, wherein the compound is a compound of the following formula (28) or (29): 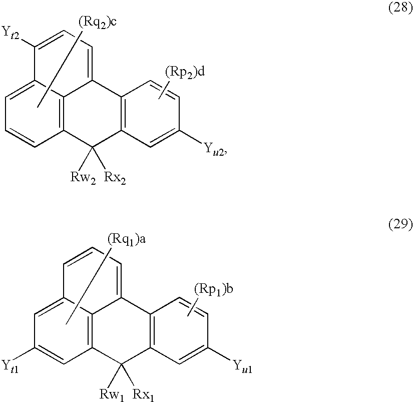
wherein Rₚ₁, R_{q1}, Rₚ₂ and R_{q2} represent each independently an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group or cyano group, a and c represent each independently an integer of 0 to 5, b and d represent each independently an integer of 0 to 3, and when there are two or more Rₚ₁s , R_{q1}s, Rₚ₂s or R_{q2}s, they may be the same or different, R_{w1}, Rₓ₁, R_{w2} and Rₓ₂ represent each independently an alkyl group, alkoxy group, alkylthio group, aryl group, aryloxy group, arylthio group, arylalkyl group, arylalkoxy group, arylalkylthio group, arylalkenyl group, arylalkynyl group, amino group, substituted amino group, silyl group, substituted silyl group, halogen atom, acyl group, acyloxy group, imine residue, amide group, acid imide group, monovalent heterocyclic group, carboxyl group, substituted carboxyl group or cyano group each having a carbon number of 3 or more, and, Rₚ₁ and R_{q1}, Rₚ₂ and R_{q2}, R_{w1} and Rₓ₁, and R_{w2} and Rₓ₂ each may be mutually connected to form a ring, Y_{t 1}, Y_{u 1}, Y_{t 2} and Y_{u 2} represent each independently a halogen atom, thiol group, alkoxy group, phenoxy group, alkylphenoxy group, alkyl sulfonate group, aryl sulfonate group, aryl alkyl sulfonate group, borate group, sulfoniummethyl group, phosphoniummethyl group, phosphonatemethyl group, monohalogenated methyl group, boric group, formyl group, cyano group, vinyl group or triazene group.

24. The compound according to claim 22, wherein Y_{t 1}, Y_{u 1}, Y_{t 2} and Y_{u 2} represent each independently a halogen atom, alkyl sulfonate group, borate group, boric group or triazene group.

25. A method for producing a conjugated polymer compound as described in claim 2, comprising polymerizing a compound of any one of said formulae (27) to (29) as one of raw materials.

26. The production method according to claim 25, comprising polymerization with a compound of any one of the following formulae (30) to (33), in addition to the compound of said formula (28) and/or (29):
Y₇—Ar₁—Y₈ (30)
Y₉—(Ar₂—X₁)_{ff}—Ar₃—Y₁₀ (31)
Y₁₁—Ar₄—X₂—Y₁₂ (32)
Y₁₃—X₃—Y₁₄ (33)
wherein Ar₁, Ar₂, Ar₃, Ar₄, ff, X₁, X₂ and X₃ represent the same meanings as described above, and Y₇, Y₈, Y₉, Y₁₀, Y₁₁, Y₁₂, Y₁₃ and Y₁₄ represent each independently a substituent participating in polymerization.

27. The production method according to claim 26, wherein the substituents participating in polymerization are selected each independently from halogen atoms, alkyl sulfonate groups, aryl sulfonate groups and aryl alkyl sulfonate groups, and polymerization is performed in the presence of a nickel zero-valent complex.

28. The production method according to claim 24, wherein the substituents participating in polymerization are selected each independently from halogen atoms, alkyl sulfonate groups, aryl sulfonate groups, aryl alkyl sulfonate groups, boric group or borate groups, the ratio of the sum of the mol numbers of halogen atoms, alkyl sulfonate groups, aryl sulfonate groups and aryl alkyl sulfonate groups in all raw material compounds to the sum of the mol numbers of a boric group and borate groups is substantially 1, and polymerization is performed using a nickel or palladium catalyst.

29. A polymer composition comprising at least one material selected from hole transporting materials, electron transporting materials and light emitting materials, and a conjugated polymer compound as described in claim 1.

30. A polymer composition comprising two or more of conjugated polymer compounds as described in claim 1.

31. A solution comprising a conjugated polymer compound as described in claim 1.

32. The solution according to claim 31, wherein the viscosity thereof is 1 to 20 mPa·s at 25° C.

33. A light emitting thin film comprising a conjugated polymer compound as described in claim 1.

34. An electric conductive thin film comprising a conjugated polymer compound as described in claim 1.

35. An organic semiconductor thin film comprising a conjugated polymer compound as described in claim 1.

36. An organic transistor having an organic semiconductor thin film as described in claim 15.

37. A polymer light emitting device having an organic layer between electrodes composed of an anode and a cathode, wherein the organic layer comprises a conjugated polymer compound as described in claim 1.

38. The polymer light emitting device according to claim 37, wherein the organic layers is a light emitting layer.

39. The polymer light emitting device according to claim 38, wherein the light emitting layer further comprises a hole transporting material, electron transporting material or light emitting material.

40. The polymer light emitting device according to claim 39, having a light emitting layer and a charge transporting layer between electrodes composed of an anode and a cathode, wherein the charge transporting layer comprises a conjugated polymer compound as described in claim 1.

41. The polymer light emitting device according to claim 40, having a light emitting layer and a charge transporting layer between electrodes composed of an anode and a cathode and having a charge injection layer between the charge transporting layer and the electrode, wherein the charge injection layer comprises a conjugated polymer compound as described in claim 1.

42. A sheet light source using a polymer light emitting device as described in claim 37.

43. A segment display using a polymer light emitting device as described in claim 37.

44. A dot matrix display using a polymer light emitting device as described in claim 37.

45. A liquid crystal display using a polymer light emitting device as described in claim 37 as back light.
